(19) United States
(12) Patent Application Publication
(10) Pub. No.: US 2009/0220991 A1
(43) Pub. Date: Sep. 3, 2009

(54) Reagents for the detection of protein phosphorylation in leukemia signaling pathways

(75) Inventors: Roberto Polakiewicz, Lexington, MA (US); Valerie Goss, Seabrook, NH (US); Albrecht Moritz, Salem, MA (US); Ting-Lei Gu, Woburn, MA (US); Kimberly Lee, Seattle, WA (US)

Correspondence Address: Nancy Chiu Wilker, Ph.D.;Chief Intellectual Property Counsel, CELL SIGNALING TECHNOLOGY, INC., 3 Trask Lane, Danvers, MA, 01923, US

(73) Assignee: CELL SIGNALING TECHNOLOGY, INC.

(21) Appl. No.: 12/074,214

(22) Filed: Feb. 29, 2008

(51) Int. Cl. G01N 33/53; G01N 33/536; C07K 16/18; C12N 5/18

(52) U.S. Cl. 435/7.8; 436/536; 530/387.7; 435/330

(57) ABSTRACT

The invention discloses nearly 480 novel phosphorylation sites identified in signal transduction proteins and pathways underlying human Leukemia, and provides phosphorylation site specific antibodies and heavy-isotope labeled peptides (AQUA peptides) for the selective detection and quantification of these phosphorylated sites/proteins, as well as methods of using the reagents for such purpose. Among the phosphorylation sites identified are sites occurring in the following protein types: adaptor/scaffold proteins, acetyltransferases, actin binding proteins, adhesion proteins, apoptosis proteins, calcium-binding proteins, cell cycle regulation proteins, cell surface proteins, channel proteins, chaperone proteins, contractile proteins, cytokine proteins, cytoskeletal proteins, G protein regulators and GTPase activating proteins, guanine nucleotide exchange factors, helicase proteins, immunoglobulin superfamily proteins, inhibitor proteins, protein kinases, lipid kinases, ligases, lipid binding proteins, methytransferases, motor proteins, oxidoreductases, phosphotases, phosphodiesterases, phospholipases, proteases, receptor proteins, trascription factors, transferases, translation/transporter proteins, and ubiquitin conjugating system proteins.

### RELATED APPLICATIONS

This application claims the benefit of, and priority to, PCT serial number PCT/US06/034126, filed Aug. 30, 2006, presently pending, the disclosure of which is incorporated herein, in its entirety, by reference.

### FIELD OF THE INVENTION

The invention relates generally to antibodies and peptide reagents for the detection of protein phosphorylation, and to protein phosphorylation in cancer.

### BACKGROUND OF THE INVENTION

The activation of proteins by post-translational modification is an important cellular mechanism for regulating most aspects of biological organization and control, including growth, development, homeostasis, and cellular communication. Protein phosphorylation, for example, plays a critical role in the etiology of many pathological conditions and diseases, including cancer, developmental disorders, autoimmune diseases, and diabetes. Yet, in spite of the importance of protein modification, it is not yet well understood at the molecular level, due to the extraordinary complexity of signaling pathways, and the slow development of technology necessary to unravel it.

Protein phosphorylation on a proteome-wide scale is extremely complex as a result of three factors: the large number of modifying proteins, e.g. kinases, encoded in the genome, the much larger number of sites on substrate proteins that are modified by these enzymes, and the dynamic nature of protein expression during growth, development, disease states, and aging. The human genome, for example, encodes over 520 different protein kinases, making them the most abundant class of enzymes known. See Hunter, Nature 411: 355-65 (2001). Most kinases phosphorylate many different substrate proteins, at distinct tyrosine, serine, and/or threonine residues. Indeed, it is estimated that one-third of all proteins encoded by the human genome are phosphorylated, and many are phosphorylated at multiple sites by different kinases. See Graves et al., Pharmacol. Ther. 82:111-21 (1999).

Many of these phosphorylation sites regulate critical biological processes and may prove to be important diagnostic or therapeutic targets for molecular medicine. For example, of the more than 100 dominant oncogenes identified to date, 46 are protein kinases. See Hunter, supra. Understanding which proteins are modified by these kinases will greatly expand our understanding of the molecular mechanisms underlying oncogenic transformation. Therefore, the identification of, and ability to detect, phosphorylation sites on a wide variety of cellular proteins is crucially important to understanding the key signaling proteins and pathways implicated in the progression of diseases like cancer.

One form of cancer in which underlying signal transduction events are involved, but still poorly understood, is leukemia. Leukemia is a malignant disease of the bone marrow and blood, characterized by abnormal accumulation of blood cells, and is divided in four major categories. An estimated 33,500 new cases of leukemia will be diagnosed in the U.S. alone this year, affecting roughly 30,000 adults and 3,000 children, and close to 24,000 patients will die from the disease (Source: The Leukemia & Lymphoma Society (2004)). Depending of the cell type involved and the rate by which the disease progresses it can be defined as acute or chronic myelogenous leukemia (AML or CML), or acute and chronic lymphocytic leukemia (ALL or CLL). The acute forms of the disease rapidly progress, causing the accumulation of immature, functionless cells in the marrow and blood, which in turn results in anemia, immunodeficiency and coagulation deficiencies, respectively. Chronic forms of leukemia progress more slowly, allowing a greater number of mature, functional cells to be produced, which amass to high concentration in the blood over time.

More than half of adult leukemias occur in patients 67 years of age or older, and leukemia accounts for about 30% of all childhood cancers. The most common type of adult leukemia is acute myelogenous leukemia (AML), with an estimated 11,920 new cases annually. Without treatment patients rarely survive beyond 6-12 months, and despite continued development of new therapies, it remains fatal in 80% of treated patients (Source: The Leukemia & Lymphoma Society (2004)). The most common childhood leukemia is acute lymphocytic leukemia (ALL), but it can develop at any age. Chronic lymphocytic leukemia (CLL) is the second most prevalent adult leukemia, with approximately 8,200 new cases of CLL diagnosed annually in the U.S. The course of the disease is typically slower than acute forms, with a five-year relative survival of 74%. Chronic myelogenous leukemia (CML) is less prevalent, with about 4,600 new cases diagnosed each year in the U.S., and is rarely observed in children.

Most varieties of leukemia are generally characterized by genetic alterations associated with the etiology of the disease, and it has recently become apparent that, in many instances, such alterations (chromosomal translocations, deletions or point mutations) result in the constitutive activation of protein kinase genes, and their products, particularly tyrosine kinases. The most well known alteration is the oncogenic role of the chimeric BCR-Abl gene, which is generated by translocation of chromosome 9 to chromosome 22, creating the so-called Philadelphia chromosome characteristic of CML (see Nowell, Science 132: 1497 (1960)). The resulting BCR-Abl kinase protein is constitutively active and elicits characteristic signaling pathways that have been shown to drive the proliferation and survival of CML cells (see Daley, Science 247: 824-830 (1990); Raitano et al., Biochim. Biophys. Acta. December 9; 1333(3): F201-16 (1997)). The recent success of Imanitib (also known as ST1571 or Gleevec®), the first molecularly targeted compound designed to specifically inhibit the tyrosine kinase activity of BCR-Abl, provided critical confirmation of the central role of BCR-Abl signaling in the progression of CML (see Schindler et al., Science 289: 1938-1942 (2000); Nardi et al., Curr. Opin. Hematol. 11: 35-43 (2003)).

The success of Gleevec® now serves as a paradigm for the development of targeted drugs designed to block the activity of other tyrosine kinases known to be involved in leukemias and other malignancies (see, e.g., Sawyers, Curr. Opin. Genet. Dev. February; 12(1): 111-5 (2002); Druker, Adv. Cancer Res. 91:1-30 (2004)). For example, recent studies have demonstrated that mutations in the FLT3 gene occur in one third of adult patients with AML. FLT3 (Fms-like tyrosine kinase 3) is a member of the class III receptor tyrosine kinase (RTK) family including FMS, platelet-derived growth factor receptor (PDGFR) and c-KIT (see Rosnet et al., Crit. Rev. Oncog. 4: 595-613 (1993). In 20-27% of patients with AML, an internal tandem duplication in the juxta-membrane region of FLT3 can be detected (see Yokota et al., Leukemia 11: 1605-1609 (1997)). Another 7% of patients have mutations within the active loop of the second kinase domain, predominantly substitutions of aspartate residue 835 (D835), while additional mutations have been described (see Yamamoto et al., Blood 97: 2434-2439 (2001); Abu-Duhier et al., Br. J. Haematol. 113: 983-988 (2001)). Expression of mutated FLT3 receptors results in constitutive tyrosine phosphorylation of FLT3, and subsequent phosphorylation and activation of downstream molecules such as STAT5, Akt and MAPK, resulting in factor-independent growth of hematopoietic cell lines.

Altogether, FLT3 is the single most common activated gene in AML known to date. This evidence has triggered an intensive search for FLT3 inhibitors for clinical use leading to at least four compounds in advanced stages of clinical development, including: PKC412 (by Novartis), CEP-701 (by Cephalon), MLN518 (by Millenium Pharmaceuticals), and SU5614 (by Sugen/Pfizer) (see Stone et al., Blood (in press)(2004); Smith et al., Blood 103: 3669-3676 (2004); Clark et al., Blood 104: 2867-2872 (2004); and Spiekerman et al., Blood 101: 1494-1504 (2003)).

There is also evidence indicating that kinases such as FLT3, c-KIT and Abl are implicated in some cases of ALL (see Cools et al., Cancer Res. 64: 6385-6389 (2004); Hu, Nat. Genet. 36:453461 (2004); and Graux et al., Nat. Genet. 36: 1084-1089 (2004)). In contrast, very little is know regarding any causative role of protein kinases in CLL, except for a high correlation between high expression of the tyrosine kinase ZAP70 and the more aggressive form of the disease (see Rassenti et al., N. Eng. J. Med. 351: 893-901 (2004)).

Despite the identification of a few key molecules involved in progression of leukemia, the vast majority of signaling protein changes underlying this disease remains unknown. There is, therefore, relatively scarce information about kinase-driven signaling pathways and phosphorylation sites relevant to the different types of leukemia. This has hampered a complete and accurate understanding of how protein activation within signaling pathways is driving these complex cancers. Accordingly, there is a continuing and pressing need to unravel the molecular mechanisms of kinase-driven oncogenesis in leukemia by identifying the downstream signaling proteins mediating cellular transformation in this disease. Identifying particular phosphorylation sites on such signaling proteins and providing new reagents, such as phospho-specific antibodies and AQUA peptides, to detect and quantify them remains particularly important to advancing our understanding of the biology of this disease.

Presently, diagnosis of leukemia is made by tissue biopsy and detection of different cell surface markers. However, misdiagnosis can occur since some leukemia cases can be negative for certain markers, and because these markers may not indicate which genes or protein kinases may be deregulated. Although the genetic translocations and/or mutations characteristic of a particular form of leukemia can be sometimes detected, it is clear that other downstream effectors of constitutively active kinases having potential diagnostic, predictive, or therapeutic value, remain to be elucidated. Accordingly, identification of downstream signaling molecules and phosphorylation sites involved in different types of leukemia and development of new reagents to detect and quantify these sites and proteins may lead to improved diagnostic/prognostic markers, as well as novel drug targets, for the detection and treatment of this disease.

### SUMMARY OF THE INVENTION

The invention discloses nearly 480 novel phosphorylation sites identified in signal transduction proteins and pathways underlying human Leukemias and provides new reagents, including phosphorylation-site specific antibodies and AQUA peptides, for the selective detection and quantification of these phosphorylated sites/proteins. Also provided are methods of using the reagents of the invention for the detection, quantification, and profiling of the disclosed phosphorylation sites.

### BRIEF DESCRIPTION OF THE DRAWINGS

FIG. 1—Is a diagram broadly depicting the immunoaffinity isolation and mass-spectrometric characterization methodology (IAP) employed to identify the novel phosphorylation sites disclosed herein.

FIG. 2—Is a table (corresponding to Table 1) enumerating the Leukemia signaling protein phosphorylation sites disclosed herein: Column A=the name of the parent protein; Column B=the SwissProt accession number for the protein (human sequence); Column C=the protein type/classification; Column D=the tyrosine or serine residue (in the parent protein amino acid sequence) at which phosphorylation occurs within the phosphorylation site; Column E=the phosphorylation site sequence encompassing the phosphorylatable residue (residue at which phosphorylation occurs (and corresponding to the respective entry in Column D) appears in lowercase; Column F=the type of leukemia in which the phosphorylation site was discovered; and Column G=the cell type(s), tissue(s) and/or patient tissue(s) in which the phosphorylation site was discovered.

FIG. 3—is an exemplary mass spectrograph depicting the detection of the tyrosine 330 phosphorylation site in DOK2 (see Row 24 in FIG. 2/Table 1), as further described in Example 1 (red and blue indicate ions detected in MS/MS spectrum); Y* indicates the phosphorylated tyrosine (shown as lowercase “y” in FIG. 2).

FIG. 4—is an exemplary mass spectrograph depicting the detection of the tyrosine 630 phosphorylation site in FLT3 (see Row 286 in FIG. 2/Table 1), as further described in Example 1 (red and blue indicate ions detected in MS/MS spectrum); Y* indicates the phosphorylated tyrosine (shown as lowercase “y” in FIG. 2).

FIG. 5—is an exemplary mass spectrograph depicting the detection of the tyrosine 1736 phosphorylation site in TSC2 (see Row 87 in FIG. 2/Table 1), as further described in Example 1 (red and blue indicate ions detected in MS/MS spectrum); Y* indicates the phosphorylated tyrosine (shown as lowercase “y” in FIG. 2).

FIG. 6—is an exemplary mass spectrograph depicting the detection of the tyrosine 260 phosphorylation site in ICAM (see Row 71 in FIG. 2/Table 1), as further described in Example 1 (red and blue indicate ions detected in MS/MS spectrum); Y* indicates the phosphorylated tyrosine (shown as lowercase “y” in FIG. 2).

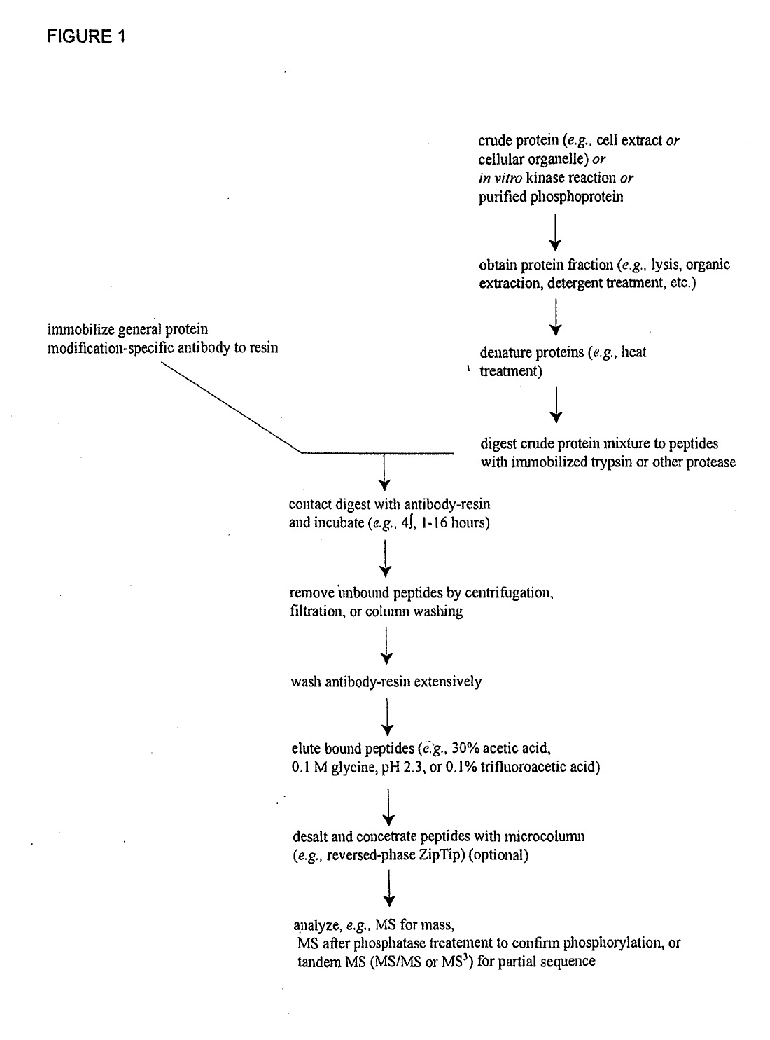

### DETAILED DESCRIPTION OF THE INVENTION

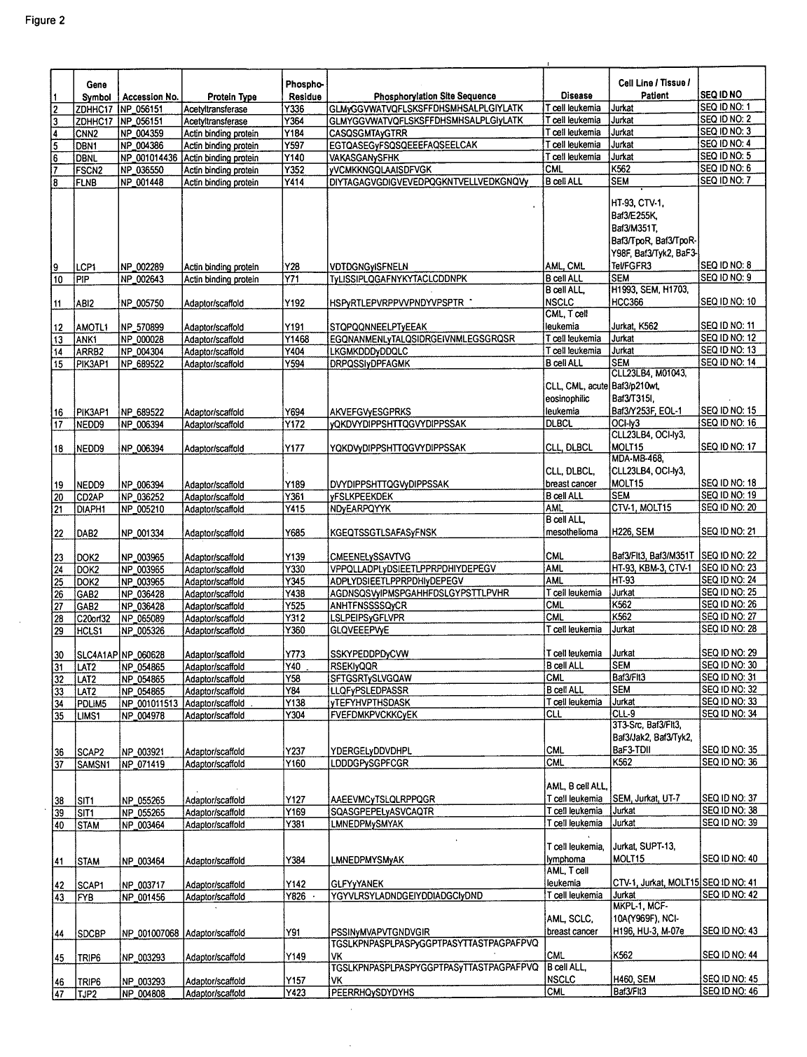

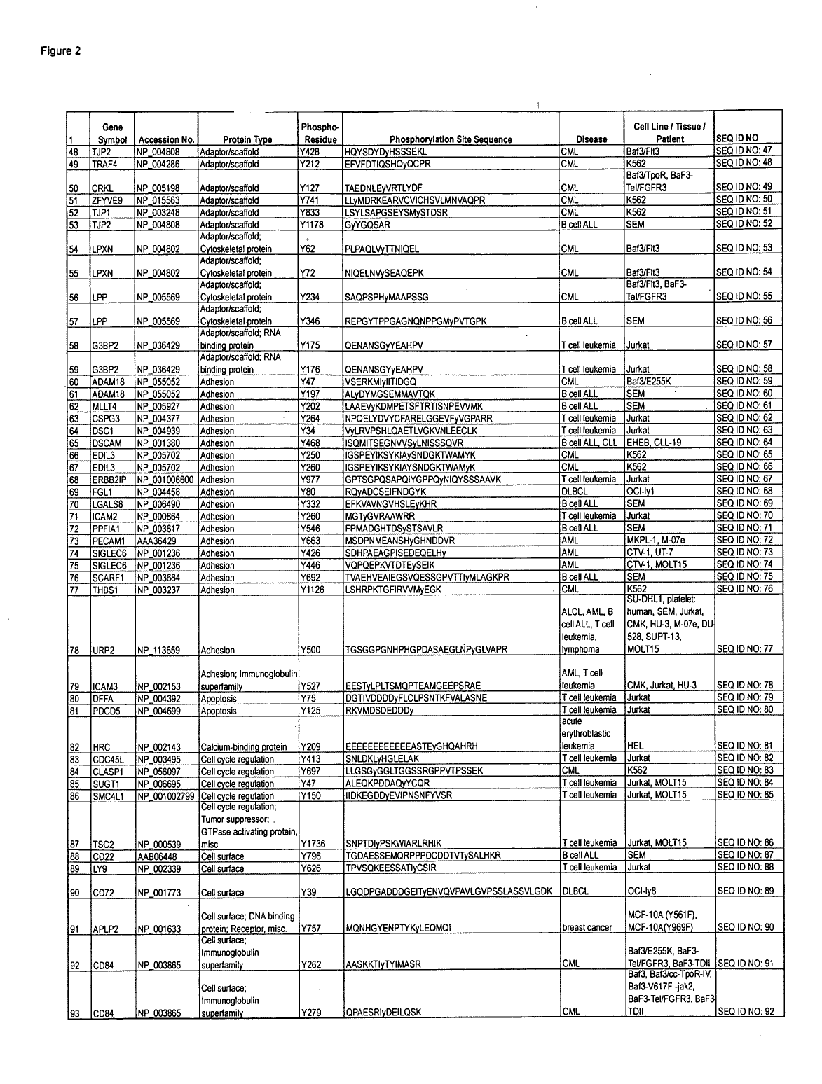

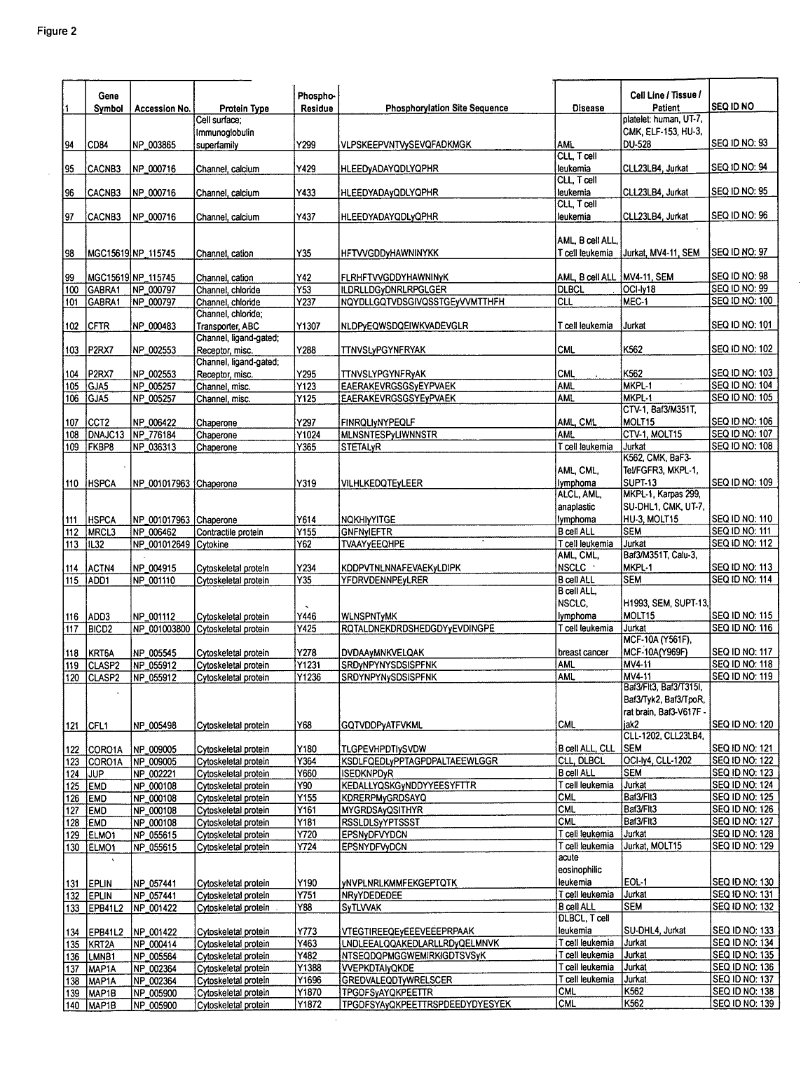

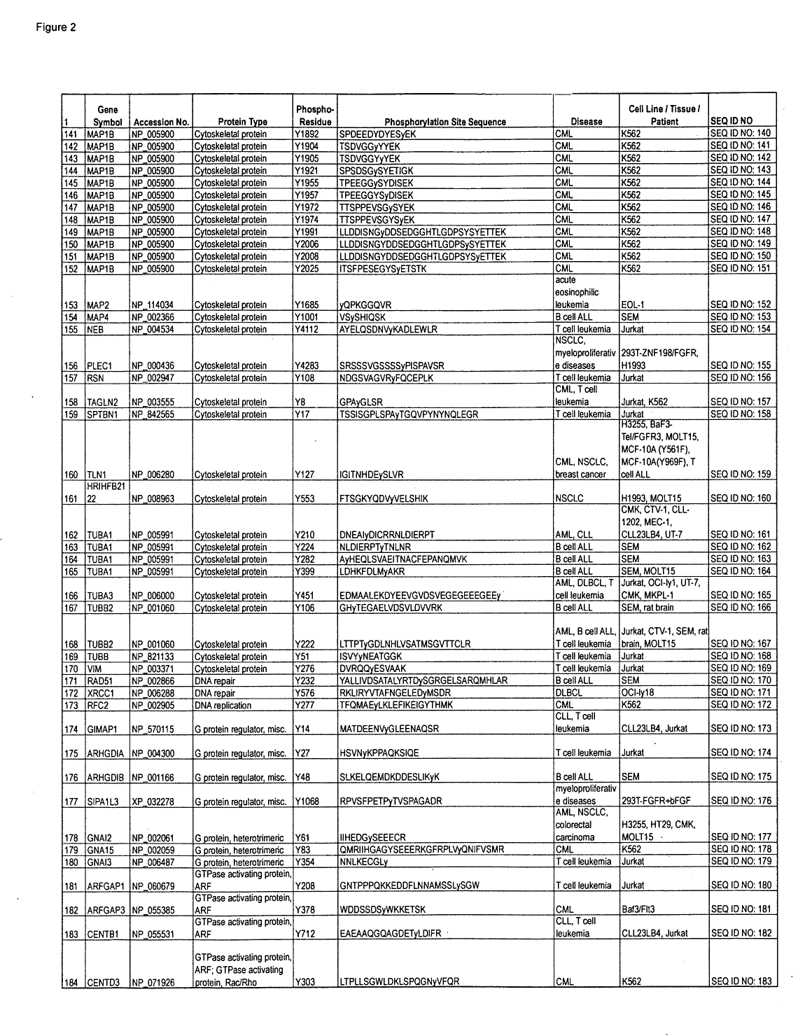

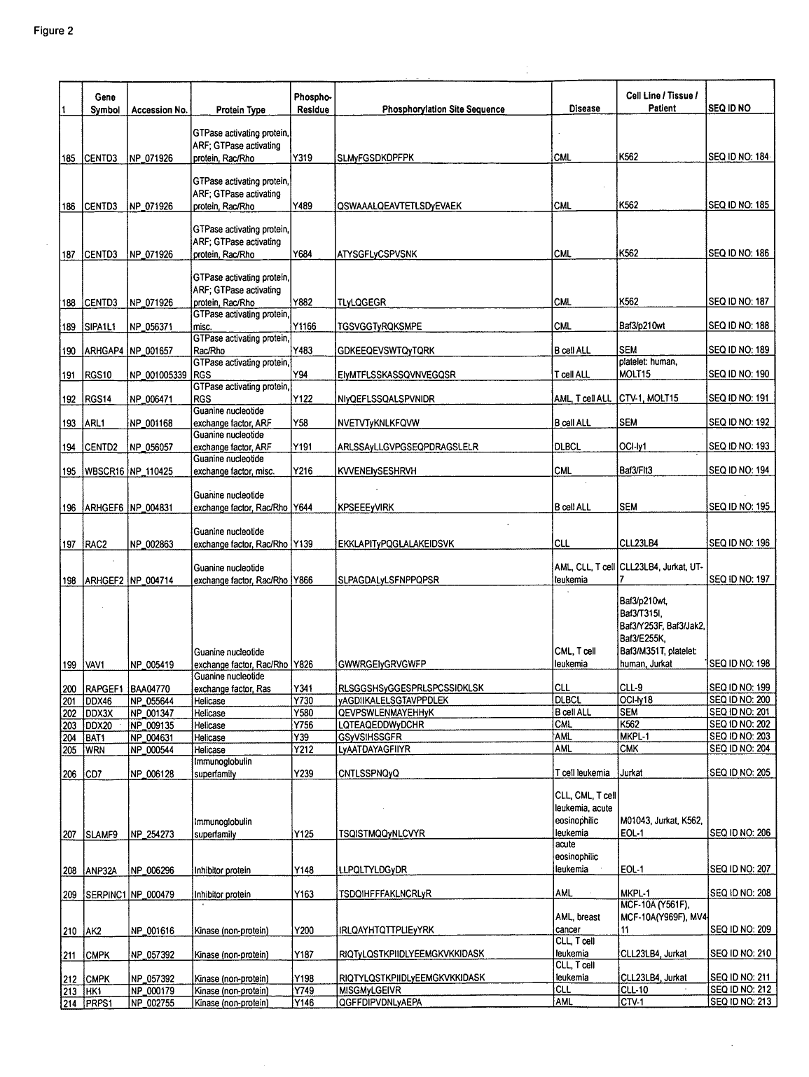

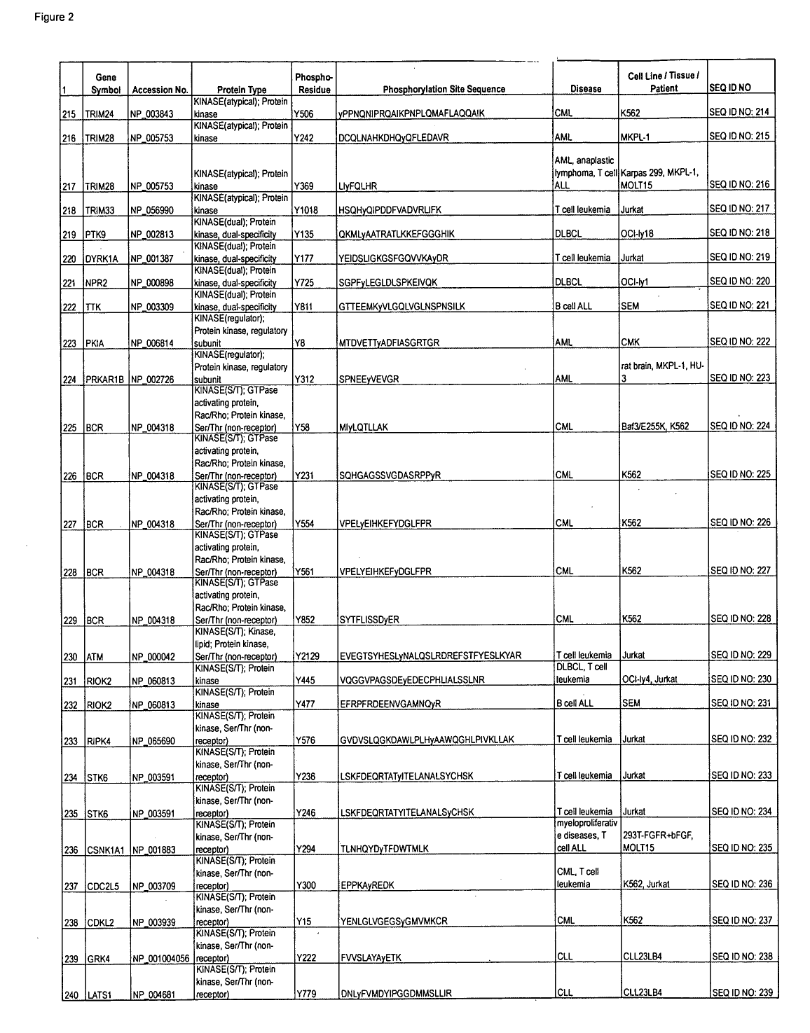

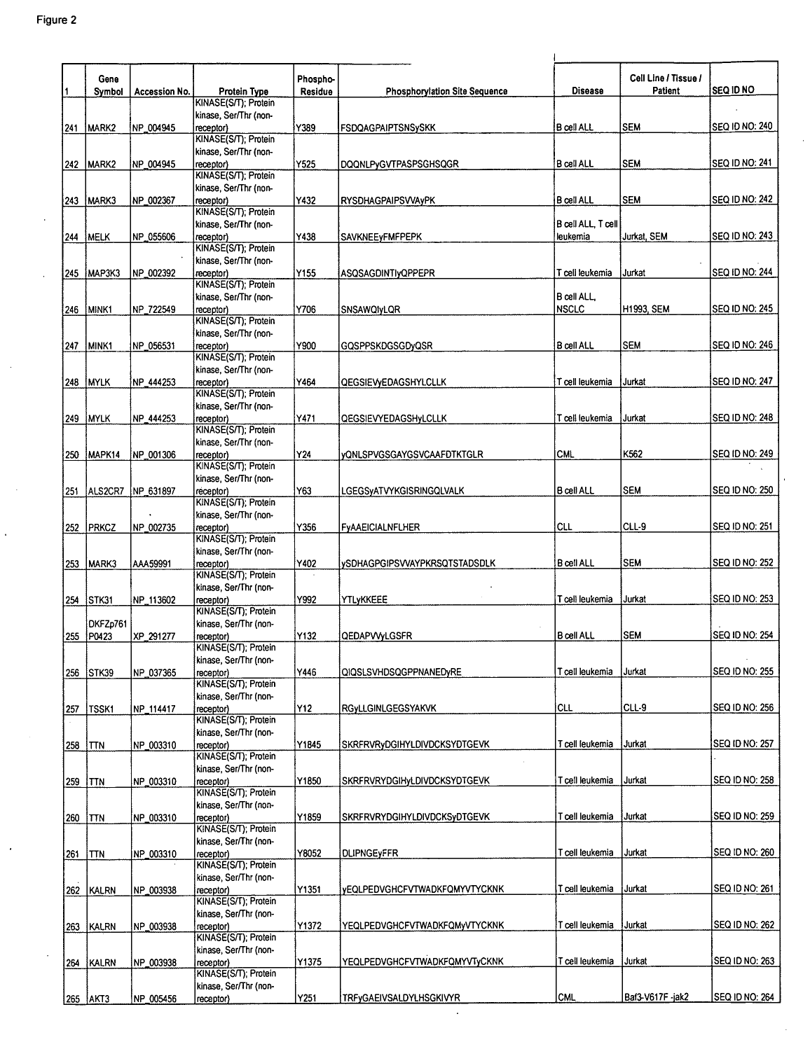

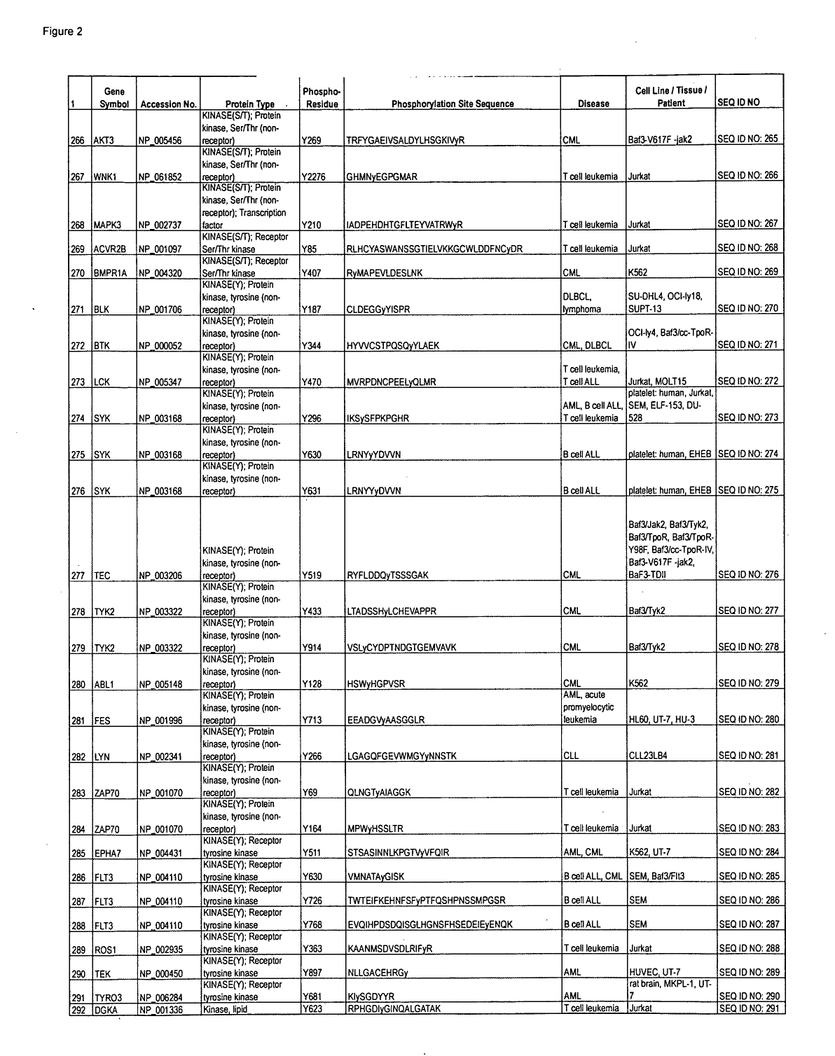

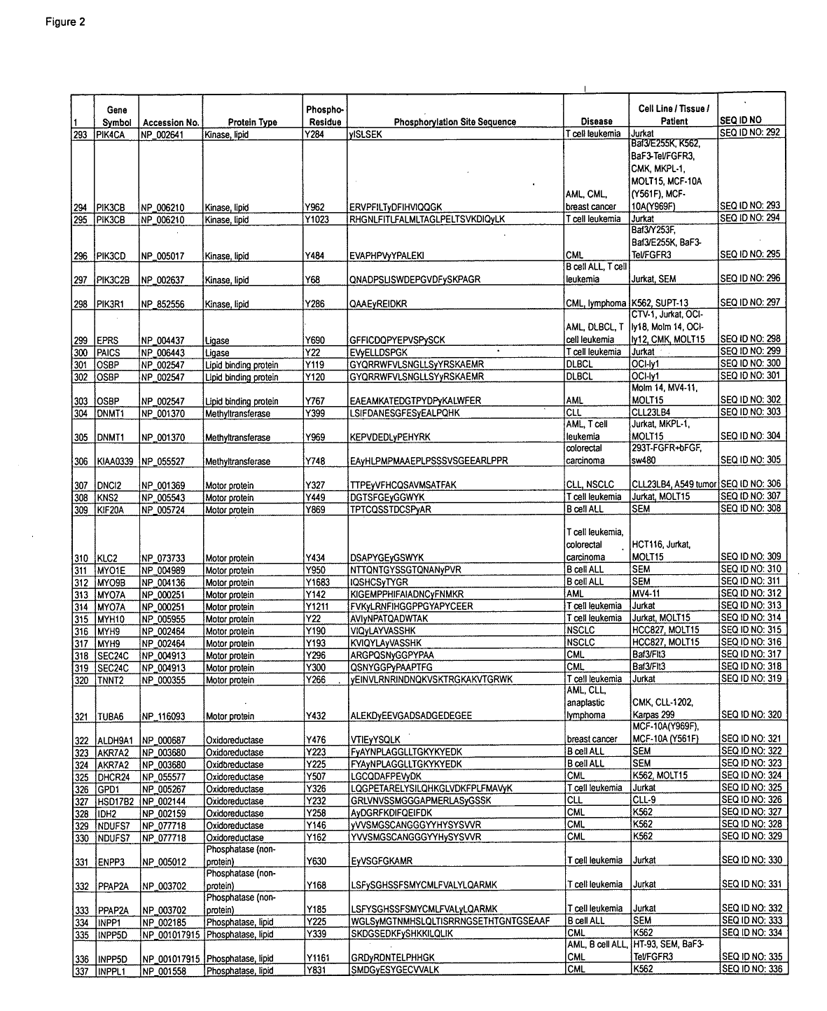

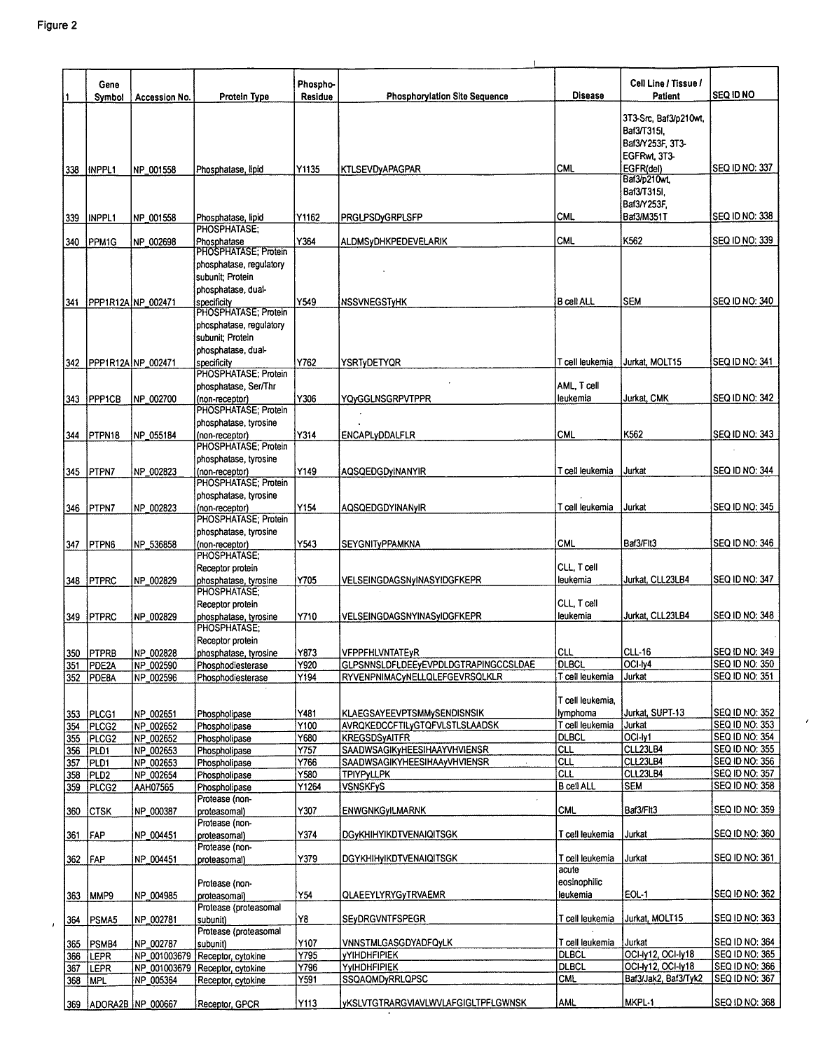

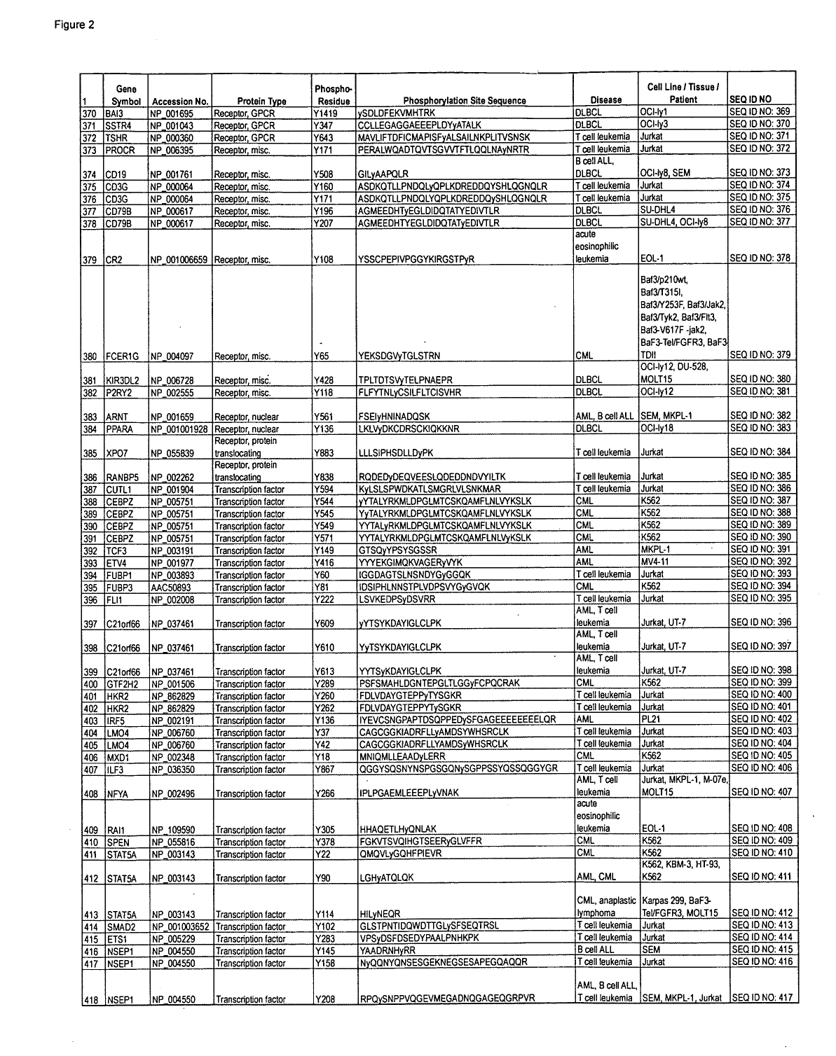

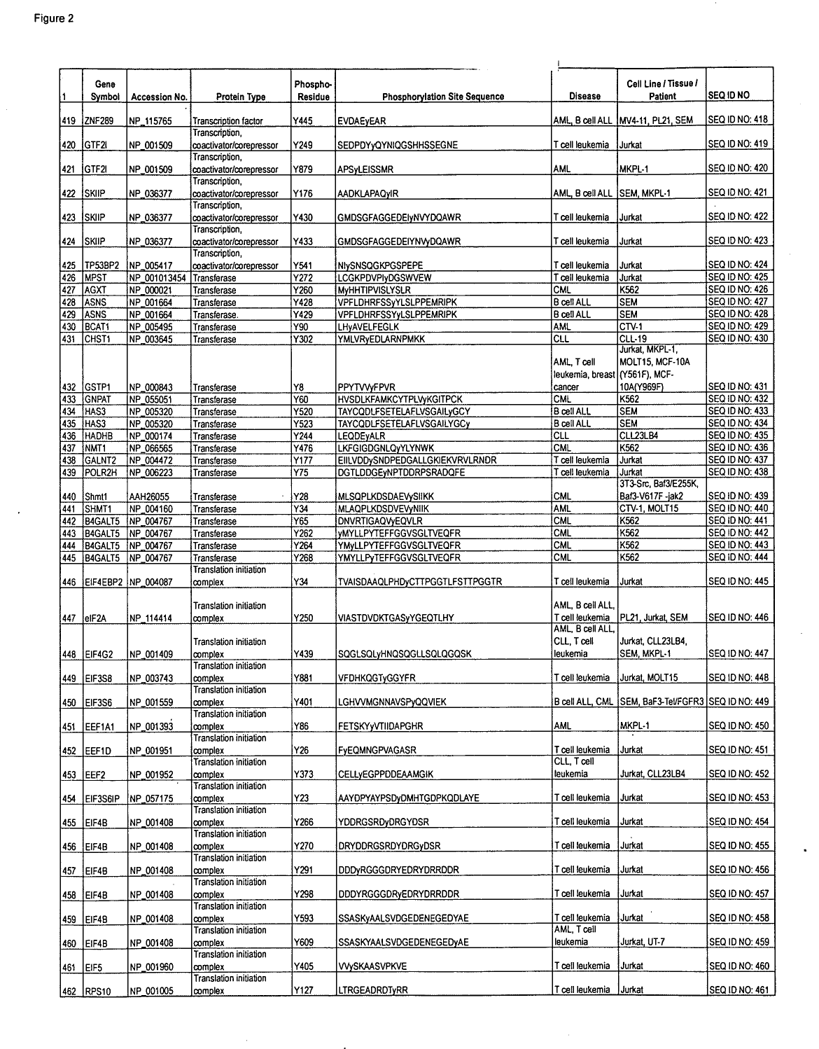

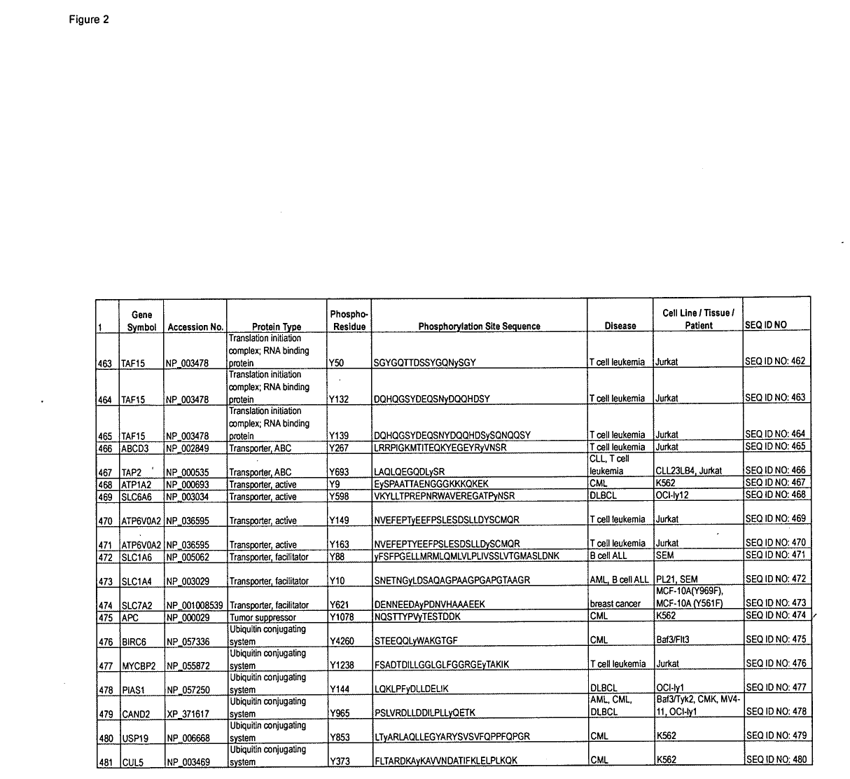

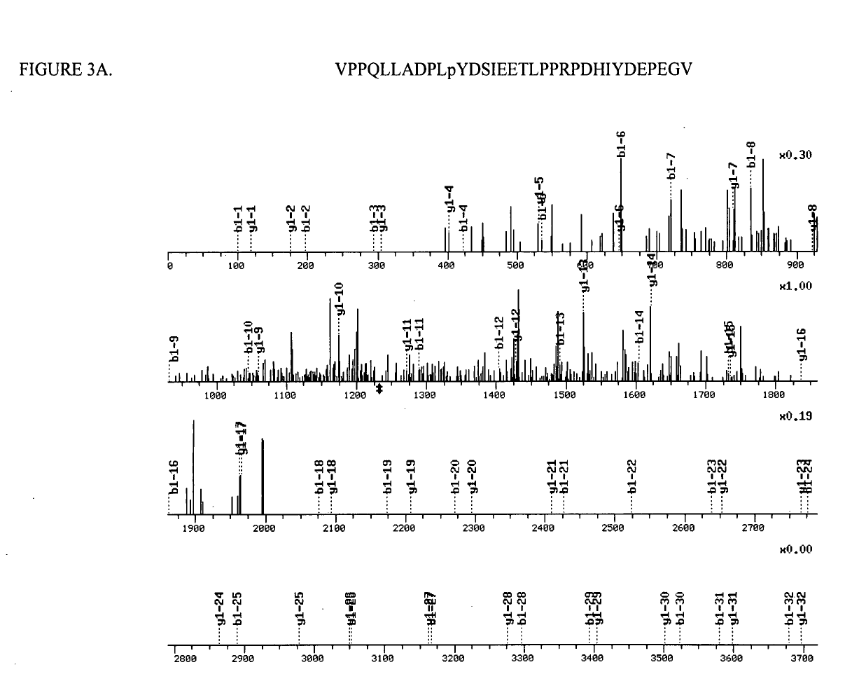

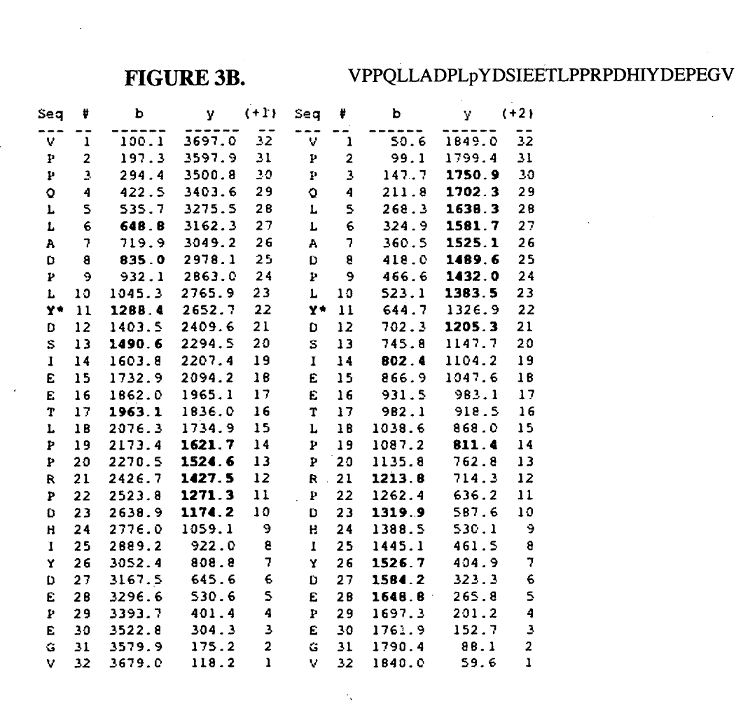

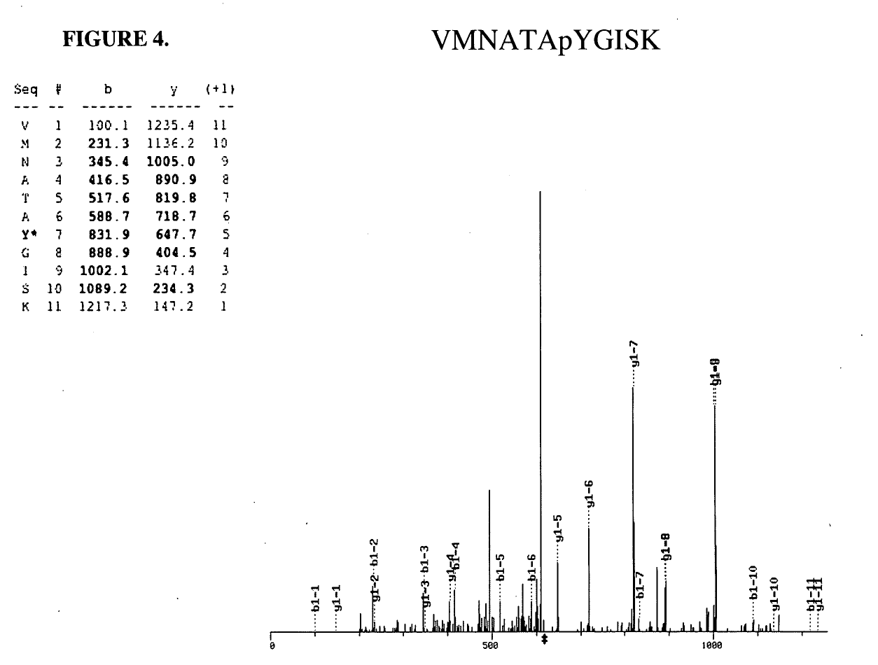

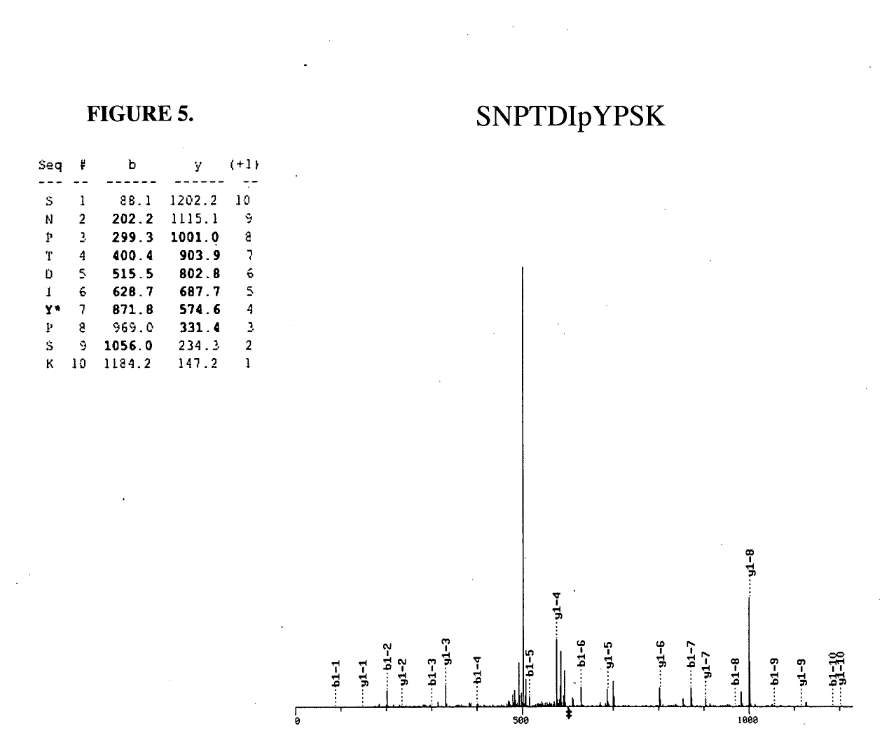

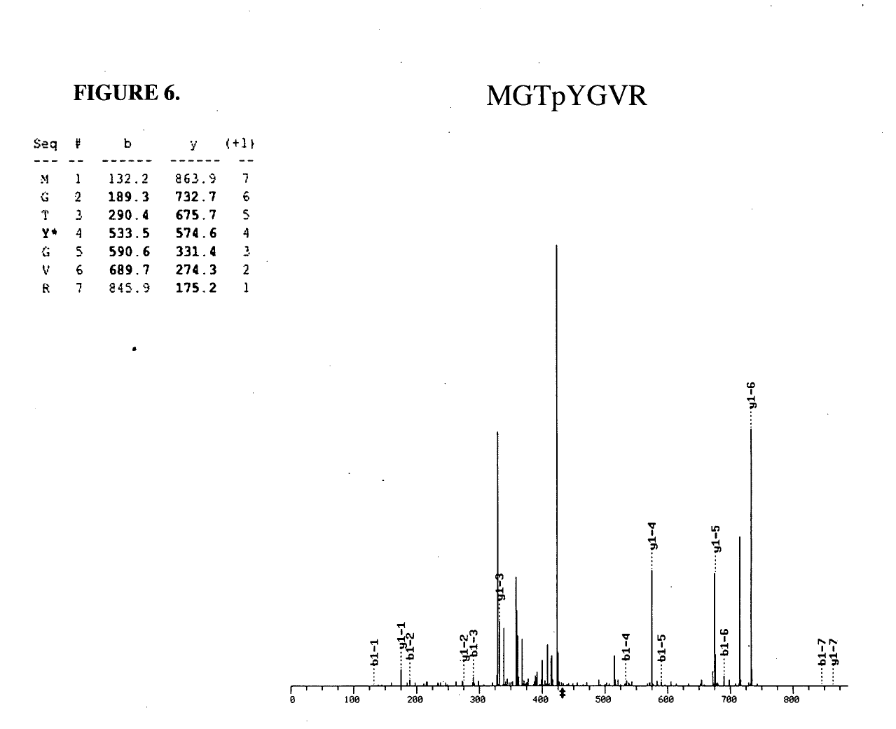

In accordance with the present invention, nearly 480 novel protein phosphorylation sites in signaling proteins and pathways underlying human Leukemia have now been discovered. These newly described phosphorylation sites were identified by employing the techniques described in “Immunoaffinity Isolation of Modified Peptides From Complex Mixtures,” U.S. Patent Publication No. 20030044848, Rush et al., using cellular extracts from a variety of leukemia-derived cell lines, e.g. SEM, HT-93, etc., as further described below. The novel phosphorylation sites (tyrosine), and their corresponding parent proteins, disclosed herein are listed in Table 1. These phosphorylation sites correspond to numerous different parent proteins (the full sequences of which (human) are all publicly available in SwissProt database and their Accession numbers listed in Column B of Table 1/FIG. 2), each of which fall into discrete protein type groups, for example Adaptor/Scaffold proteins, Cytoskeletal proteins, Protein Kinases, and Adhesion proteins, etc. (see Column C of Table 1), the phosphorylation of which is relevant to signal transduction activity underlying Leukemias (AML, CML, CLL, and ALL), as disclosed herein.

The discovery of the nearly 480 novel protein phosphorylation sites described herein enables the production, by standard methods, of new reagents, such as phosphorylation site-specific antibodies and AQUA peptides (heavy-isotope labeled peptides), capable of specifically detecting and/or quantifying these phosphorylated sites/proteins. Such reagents are highly useful, inter alia, for studying signal transduction events underlying the progression of Leukemia. Accordingly, the invention provides novel reagents—phospho-specific antibodies and AQUA peptides—for the specific detection and/or quantification of a Leukemia-related signaling protein/polypeptide only when phosphorylated (or only when not phosphorylated) at a particular phosphorylation site disclosed herein. The invention also provides methods of detecting and/or quantifying one or more phosphorylated Leukemia-related signaling proteins using the phosphorylation-site specific antibodies and AQUA peptides of the invention, and methods of obtaining a phosphorylation profile of such proteins (e.g. Kinases).

In part, the invention provides an isolated phosphorylation site-specific antibody that specifically binds a given Leukemia-related signaling protein only when phosphorylated (or not phosphorylated, respectively) at a particular tyrosine enumerated in Column D of Table 1/FIG. 2 comprised within the phosphorylatable peptide site sequence enumerated in corresponding Column E. In further part, the invention provides a heavy-isotope labeled peptide (AQUA peptide) for the detection and quantification of a given Leukemia-related signaling protein, the labeled peptide comprising a particular phosphorylatable peptide site/sequence enumerated in Column E of Table 1/FIG. 2 herein. For example, among the reagents provided by the invention is an isolated phosphorylation site-specific antibody that specifically binds the MELK tyrosine kinase only when phosphorylated (or only when not phosphorylated) at tyrosine 438 (see Row 244 (and Columns D and E) of Table 1/FIG. 2). By way of further example, among the group of reagents provided by the invention is an AQUA peptide for the quantification of phosphorylated MELK tyrosine kinase, the AQUA peptide comprising the phosphorylatable peptide sequence listed in Column E, Row 244, of Table 1/FIG. 2 (which encompasses the phosphorylatable tyrosine at position 438).

In one embodiment, the invention provides an isolated phosphorylation site-specific antibody that specifically binds a human Leukemia-related signaling protein selected from Column A of Table 1 (Rows 2-481) only when phosphorylated at the tyrosine residue listed in corresponding Column D of Table 1, comprised within the phosphorylatable peptide sequence listed in corresponding Column E of Table 1 (SEQ ID NOs: 1-7, 9-14, 16, 19-21, 23, 26-30, 32-34, 36-45, 48-52, 56-58, 60-90, 93-119, 121-124, 129-151, 153-160, 163-180, 182-193, 195-197, 199-208, 210-221, 223-279, 281-294, 296-297, 299-316, 319-336, 339-345, 347-356, 358, 360-366, 368-378, 380-417, 419-438, 440-474, and 476-480), wherein said antibody does not bind said signaling protein when not phosphorylated at said tyrosine. In another embodiment, the invention provides an isolated phosphorylation site-specific antibody that specifically binds a Leukemia-related signaling protein selected from Column A of Table 1 only when not phosphorylated at the tyrosine residue listed in corresponding Column D of Table 1, comprised within the peptide sequence listed in corresponding Column E of Table 1 (SEQ ID NOs: 1-7, 9-14, 16, 19-21, 23, 26-30, 32-34, 36-45, 48-52, 56-58, 60-90, 93-119, 121-124, 129-151, 153-160, 163-180, 182-193, 195-197, 199-208, 210-221, 223-279, 281-294, 296-297, 299-316, 319-336, 339-345, 347-356, 358, 360-366, 368-378, 380-417, 419-438, 440-474, and 476-480), wherein said antibody does not bind said signaling protein when phosphorylated at said tyrosine. Such reagents enable the specific detection of phosphorylation (or non-phosphorylation) of a novel phosphorylatable site disclosed herein. The invention further provides immortalized cell lines producing such antibodies. In one preferred embodiment, the immortalized cell line is a rabbit or mouse hybridoma.

In another embodiment, the invention provides a heavy-isotope labeled peptide (AQUA peptide) for the quantification of a Leukemia-related signaling protein selected from Column A of Table 1, said labeled peptide comprising the phosphorylatable peptide sequence listed in corresponding Column E of Table 1 (SEQ ID NOs: 1-7, 9-14, 16, 19-21, 23, 26-30, 32-34, 36-45, 48-52, 56-58, 60-90, 93-119, 121-124, 129-151, 153-160, 163-180, 182-193, 195-197, 199-208, 210-221, 223-279, 281-294, 296-297, 299-316, 319-336, 339-345, 347-356, 358, 360-366,-368-378, 380-417, 419-438, 440-474, and 476-480), which sequence comprises the phosphorylatable tyrosine listed in corresponding Column D of Table 1. In certain preferred embodiments, the phosphorylatable tyrosine within the labeled peptide is phosphorylated, while in other preferred embodiments, the phosphorylatable residue within the labeled peptide is not phosphorylated.

Reagents (antibodies and AQUA peptides) provided by the invention may conveniently be grouped by the type of Leukemia-related signaling protein in which a given phosphorylation site (for which reagents are provided) occurs. The protein types for each respective protein (in which a phosphorylation site has been discovered) are provided in Column C of Table 1/FIG. 2, and include: adaptor/scaffold proteins, acetyltransferases, actin binding proteins, adhesion proteins, apoptosis proteins, calcium-binding proteins, cell cycle regulation proteins, cell surface proteins, channel proteins, chaperone proteins, contractile proteins, cytokine proteins, cytoskeletal proteins, G protein regulators and GTPase activating proteins, guanine nucleotide exchange factors, helicase proteins, immunoglobulin superfamily proteins, inhibitor proteins, protein kinases, lipid kinases, ligases, lipid binding proteins, methytransferases, motor proteins, oxidoreductases, phosphotases, phosphodiesterases, phospholipases, proteases, receptor proteins, transcription factors, transferases, translation/transporter proteins, and ubiquitin conjugating system proteins. Each of these distinct protein groups is considered a preferred subset of Leukemia-related signal transduction protein phosphorylation sites disclosed herein, and reagents for their detection/quantification may be considered a preferred subset of reagents provided by the invention.

Particularly preferred subsets of the phosphorylation sites (and their corresponding proteins) disclosed herein are those occurring on the following protein types/groups listed in Column C of Table 1/FIG. 2, are the protein kinases adaptor/scaffold proteins, adhesion proteins, cell cycle regulation proteins, cell surface proteins, transcription proteins, phosphatases, phospholipases, phosphodiesterases, receptor proteins, cytoskeltal proteins, G protein regulators, and lipid kinases. Accordingly, among preferred subsets of reagents provided by the invention are isolated antibodies and AQUA peptides useful for the detection and/or quantification of the foregoing preferred protein/phosphorylation site subsets.

In one subset of preferred embodiments, there is provided:
(i) An isolated phosphorylation site-specific antibody that specifically binds a protein kinase selected from Column A, Rows 210-291, of Table 1 only when phosphorylated at the tyrosine listed in corresponding Column D, Rows 210-291, of Table 1, comprised within the phosphorylatable peptide sequence listed in corresponding Column E, Rows 210-291, of Table 1 (SEQ ID NOs: 210-221, 223-280, and 281-290), wherein said antibody does not bind said protein when not phosphorylated at said tyrosine.
(ii) An equivalent antibody to (i) above that only binds the protein kinase when not phosphorylated at the disclosed site (and does not bind the protein when it is phosphorylated at the site).
(iii) A heavy-isotope labeled peptide (AQUA peptide) for the quantification of a protein kinase selected from Column A, Rows 210-291, said labeled peptide comprising the phosphorylatable peptide sequence listed in corresponding Column E, Rows 210-291, of Table 1 (SEQ ID NOs: 210-221, 223-280, and 281-290), which sequence comprises the phosphorylatable tyrosine listed in corresponding Column D, Rows 210-291, of Table 1.

Among this preferred subset of reagents, antibodies and AQUA peptides for the detection/quantification of the following protein kinase phosphorylation sites are particularly preferred: ATM (Y2129), MELK (Y438), MAPK14 (Y24), BLK (Y187), BTK (Y344), SYK (Y296), ZAP70 (Y69), FLT3 (Y630), FLT3 (Y726), FLT3 (Y768), and ROS1 (Y363) (see SEQ ID NOs: 229, 243, 249, 270-271, 273, 283, and 285-288).

In a second subset of preferred embodiments there is provided:
(i) An isolated phosphorylation site-specific antibody that specifically binds an adaptor/scaffold protein selected from Column A, Rows 11-59, of Table 1 only when phosphorylated at the tyrosine listed in corresponding Column D, Rows 11-59, of Table 1, comprised within the phosphorylatable peptide sequence listed in corresponding Column E, Rows 11-59, of Table 1 (SEQ ID NOs: 10-14, 16,19-21, 23, 26-30, 32-34, 36-45, 48-52, and 56-58), wherein said antibody does not bind said protein when not phosphorylated at said tyrosine.
(ii) An equivalent antibody to (i) above that only binds the adaptor/scaffold protein when not phosphorylated at the disclosed site (and does not bind the protein when it is phosphorylated at the site).
(iii) A heavy-isotope labeled peptide (AQUA peptide) for the quantification of a Leukemia-related signaling protein that is a adaptor/scaffold protein selected from Column A, Rows 11-59, said labeled peptide comprising the phosphorylatable peptide sequence listed in corresponding Column E, Rows 11-59, of Table 1 (SEQ ID NOs: 10-14, 16, 19-21, 23, 26-30, 32-34, 36-45, 48-52, and 56-58), which sequence comprises the phosphorylatable tyrosine listed in corresponding Column D, Rows 11-59, of Table 1.

Among this preferred subset of reagents, antibodies and AQUA peptides for the detection/quantification of the following adaptor/scaffold protein phosphorylation sites are particularly preferred: ABI2 (Y192), PIK3AP1 (Y594), DOK2 (Y330), LAT2 (Y40), SIT1 (Y127), STAM (Y384), SCAP1 (Y142) (see SEQ ID NOs: 10,14, 23, 30, 37, 40-41).

In another subset of preferred embodiments there is provided:
(i) An isolated phosphorylation site-specific antibody that specifically binds an adhesion protein selected from Column A, Rows 60-79, of Table 1 only when phosphorylated at the tyrosine or serine listed in corresponding Column D, Rows 60-79, of Table 1, comprised within the phosphorylatable peptide sequence listed in corresponding Column E, Rows 60-79, of Table 1 (SEQ ID NOs: 60-78), wherein said antibody does not bind said protein when not phosphorylated at said tyrosine.
(ii) An equivalent antibody to (i) above that only binds the adhesion protein when not phosphorylated at the disclosed site (and does not bind the protein when it is phosphorylated at the site).
(iii) A heavy-isotope labeled peptide (AQUA peptide) for the quantification of a Leukemia-related signaling protein that is an adhesion protein selected from Column A, Rows 60-79, said labeled peptide comprising the phosphorylatable peptide sequence listed in corresponding Column E, Rows 60-79, of Table 1 (SEQ ID NOs: 60-78), which sequence comprises the phosphorylatable tyrosine listed in corresponding Column D, Rows 60-79, of Table 1.

Among this preferred subset of reagents, antibodies and AQUA peptides for the detection/quantification of the following adhesion protein phosphorylation sites are particularly preferred: ADAM18 (Y197), ICAM2 (Y260) and PECAM1 (Y663) (see SEQ ID NOs: 60, 70 and 72).

In still another subset of preferred embodiments there is provided:
(i) An isolated phosphorylation site-specific antibody that specifically binds a cell cycle regulation protein selected from Column A, Rows 83-87, of Table 1 only when phosphorylated at the tyrosine listed in corresponding Column D, Rows 83-87, of Table 1, comprised within the phosphorylatable peptide sequence listed in corresponding Column E, Rows 83-87, of Table 1 (SEQ ID NOs: 82-86), wherein said antibody does not bind said protein when not phosphorylated at said tyrosine.
(ii) An equivalent antibody to (i) above that only binds the cell cycle regulation protein when not phosphorylated at the disclosed site (and does not bind the protein when it is phosphorylated at the site).
(iii) A heavy-isotope labeled peptide (AQUA peptide) for the quantification of a Leukemia-related signaling protein that is a cell cycle regulation protein selected from Column A, Rows 83-87, said labeled peptide comprising the phosphorylatable peptide sequence listed in corresponding Column E, Rows 83-87, of Table 1 (SEQ ID NOs: 82-86), which sequence comprises the phosphorylatable tyrosine listed in corresponding Column D, Rows 83-87, of Table 1.

Among this preferred subset of reagents, antibodies and AQUA peptides for the detection/quantification of the following cell cycle regulation protein phosphorylation sites are particularly preferred: TSC2 (Y1736) (see SEQ ID NO: 86).

In still another subset of preferred embodiments there is provided:
(i) An isolated phosphorylation site-specific antibody that specifically binds a cell surface protein selected from Column A, Rows 88-94, of Table 1 only when phosphorylated at the tyrosine listed in corresponding Column D, Rows 88-94, of Table 1, comprised within the phosphorylatable peptide sequence listed in corresponding Column E, Rows 88-94, of Table 1 (SEQ ID NOs: 87-90, and 93), wherein said antibody does not bind said protein when not phosphorylated at said tyrosine.
(ii) An equivalent antibody to (i) above that only binds the cell surface protein when not phosphorylated at the disclosed site (and does not bind the protein when it is phosphorylated at the site).
(iii) A heavy-isotope labeled peptide (AQUA peptide) for the quantification of a Leukemia-related signaling protein that is a cell surface protein selected from Column A, Rows 88-94, said labeled peptide comprising the phosphorylatable peptide sequence listed in corresponding Column E, Rows 88-94, of Table 1 (SEQ ID NOs: 87-90, and 93), which sequence comprises the phosphorylatable tyrosine listed in corresponding Column D, Rows 88-94, of Table 1.

Among this preferred subset of reagents, antibodies and AQUA peptides for the detection/quantification of the following cell surface protein phosphorylation sites are particularly preferred: CD72 (Y39) and CD84 (Y299) (see SEQ ID NOs: 89 and 93).

In still another subset of preferred embodiments there is provided:
(i) An isolated phosphorylation site-specific antibody that specifically binds a transcription factor/coactivator/corepressor selected from Column A, Rows 387-425, of Table 1 only when phosphorylated at the tyrosine listed in corresponding Column D, Rows 387-425, of Table 1, comprised within the phosphorylatable peptide sequence listed in corresponding Column E, Rows 387-425 of Table 1 (SEQ ID NOs: 386-417, and 419-424), wherein said antibody does not bind said protein when not phosphorylated at said tyrosine.
(ii) An equivalent antibody to (i) above that only binds transcription factor/coactivator/corepressor when not phosphorylated at the disclosed site (and does not bind the protein when it is phosphorylated at the site).
(iii) A heavy-isotope labeled peptide (AQUA peptide) for the quantification of a Leukemia-related signaling protein that is a transcription factor/coactivator/corepressor selected from Column A, Rows 387-425, said labeled peptide comprising the phosphorylatable peptide sequence listed in corresponding Column E, Rows 387-425, of Table 1 (SEQ ID NOs: 386-417, and 419-424), which sequence comprises the phosphorylatable tyrosine or serine listed in corresponding Column D, Rows 387-425, of Table 1.

Among this preferred subset of reagents, antibodies and AQUA peptides for the detection/quantification of the following trascription factor/coactivor/corepressor phosphorylation sites are particularly preferred: STAT5A (Y22), STAT5A (Y90), STAT5A (Y1 14), SMAD2 (Y102), and NSEP1 (Y208) (see SEQ ID NOs: 410-413 and 417).

In yet another subset of preferred embodiments, there is provided:
(i) An isolated phosphorylation site-specific antibody that specifically binds a phophatase selected from Column A, Rows 331-350, of Table 1 only when phosphorylated at the tyrosine listed in corresponding Column D, Rows 331-350, of Table 1, comprised within the phosphorylatable peptide sequence listed in corresponding Column E, Rows 331-350, of Table 1 (SEQ ID NOs: 330-349), wherein said antibody does not bind said protein when not phosphorylated at said tyrosine.
(ii) An equivalent antibody to (i) above that only binds the phosphatase when not phosphorylated at the disclosed site (and does not bind the protein when it is phosphorylated at the site).
(iii) A heavy-isotope labeled peptide (AQUA peptide) for the quantification of a Leukemia-related signaling protein that is a phophatase selected from Column A, Rows 331-350, said labeled peptide comprising the phosphorylatable peptide sequence listed in corresponding Column E, Rows 331-350, of Table 1 (SEQ ID NOs: 330-349), which sequence comprises the phosphorylatable tyrosine listed in corresponding Column D, Rows 331-350, of Table 1.

Among this preferred subset of reagents, antibodies and AQUA peptides for the detection/quantification of the following phosphatase phosphorylation sites are particularly preferred: INPPL1 (Y831), INPPL1 (Y1135) and PTPRC (Y705) (see SEQ ID NOs: 336-337, and 347).

In yet another subset of preferred embodiments, there is provided:
(i) An isolated phosphorylation site-specific antibody specifically binds a phosphodiesterase/phospholipase selected from Column A, Rows 351-359, of Table 1 only when phosphorylated at the tyrosine listed in corresponding Column D, Rows 351-359, of Table 1, comprised within the phosphorylatable peptide sequence listed in corresponding Column E, Rows 351-359, of Table 1 (SEQ ID NOs: 350-356, and 358), wherein said antibody does not bind said protein when not phosphorylated at said tyrosine.
(ii) An equivalent antibody to (i) above that only binds the Tyrosine Protein Kinase when not phosphorylated at the disclosed site (and does not bind the protein when it is phosphorylated at the site).
(iii) A heavy-isotope labeled peptide (AQUA peptide) for the quantification of a Leukemia-related signaling protein that is a phosphodiesterase/phospholipase selected from Column A, Rows 351-359, said labeled peptide comprising the phosphorylatable peptide sequence listed in corresponding Column E, Rows 351-359, of Table 1 (SEQ ID NOs: 350-356, and 358), which sequence comprises the phosphorylatable tyrosine listed in corresponding Column D, Rows 351-359, of Table 1.

Among this preferred subset of reagents, antibodies and AQUA peptides for the detection/quantification of the following phosphodiesterase/phospholipase phosphorylation sites are particularly preferred: PLCG1 (Y481), PLCG2 (Y680) and PLCG2 (Y1264) (see SEQ ID NOs: 352, 354 and 358).

In yet another subset of preferred embodiments, there is provided:
(i) An isolated phosphorylation site-specific antibody that specifically binds a receptor protein selected from Column A, Rows 366-386, of Table 1 only when phosphorylated at the tyrosine listed in corresponding Column D, Rows 366-386, of Table 1, comprised within the phosphorylatable peptide sequence listed in corresponding Column E, Rows 366-386, of Table 1 (SEQ ID NOs: 365-366, 368-378, and 380-385), wherein said antibody does not bind said protein when not phosphorylated at said tyrosine.
(ii) An equivalent antibody to (i) above that only binds the receptor protein when not phosphorylated at the disclosed site (and does not bind the protein when it is phosphorylated at the site).
(iii) A heavy-isotope labeled peptide (AQUA peptide) for the quantification of a Leukemia-related signaling protein that is a receptor protein selected from Column A, Rows 366-386, said labeled peptide comprising the phosphorylatable peptide sequence listed in corresponding Column E, Rows 366-386, of Table 1 (SEQ ID NOs: 365-366, 368-378, and 380-385), which sequence comprises the phosphorylatable tyrosine listed in corresponding Column D, Rows 366-386, of Table 1.

Among this preferred subset of reagents, antibodies and AQUA peptides for the detection/quantification of the following receptor protein phosphorylation sites are particularly preferred: LEPR (Y795) (see SEQ ID NO: 365).

In still another subset of preferred embodiments, there is provided:
(i) An isolated phosphorylation site-specific antibody that specifically binds a cytoskeletal protein selected from Column A, Rows 114-170, of Table 1 only when phosphorylated at the tyrosine listed in corresponding Column D, Rows 114-170, of Table 1, comprised within the phosphorylatable peptide sequence listed in corresponding Column E, Rows 114-170, of Table 1 (SEQ ID NOs: 113-119, 121-124, 129-151, 153-160, and 163-169), wherein said antibody does not bind said protein when not phosphorylated at said tyrosine.
(ii) An equivalent antibody to (i) above that only binds the cytoskeletal protein when not phosphorylated at the disclosed site (and does not bind the protein when it is phosphorylated at the site).
(iii) A heavy-isotope labeled peptide (AQUA peptide) for the quantification of a Leukemia-related signaling protein that cytoskeletal protein selected from Column A, Rows 114-170, said labeled peptide comprising the phosphorylatable peptide sequence listed in corresponding Column E, Rows 114-170, of Table 1 (SEQ ID NOs: 113-119, 121-124, 129-151, 153-160, and 163-169), which sequence comprises the phosphorylatable tyrosine or serine listed in corresponding Column D, Rows 114-170, of Table 1.

In still another subset of preferred embodiments, there is provided:
(i) An isolated phosphorylation site-specific antibody that specifically binds a G protein selected from Column A, Rows 174-180, of Table 1 only when phosphorylated at the tyrosine listed in corresponding Column D, Rows 174-180, of Table 1, comprised within the phosphorylatable peptide sequence listed in corresponding Column E, Rows 174-180, of Table 1 (SEQ ID NOs: 173-189), wherein said antibody does not bind said protein when not phosphorylated at said tyrosine.
(ii) An equivalent antibody to (i) above that only binds the G protein when not phosphorylated at the disclosed site (and does not bind the protein when it is phosphorylated at the site).
(iii) A heavy-isotope labeled peptide (AQUA peptide) for the quantification of a Leukemia-related signaling protein that is an G protein selected from Column A, Rows 174-180, said labeled peptide comprising the phosphorylatable peptide sequence listed in corresponding Column E, Rows 174-180, of Table 1 (SEQ ID NOs: 173-179), which sequence comprises the phosphorylatable tyrosine listed in corresponding Column D, Rows 174-180, of Table 1.

Among this preferred subset of reagents, antibodies and AQUA peptides for the detection/quantification of the following G protein phosphorylation sites are particularly preferred: GNAI2 (Y61) (see SEQ ID NO: 177).

In still another subset of preferred embodiments, there is provided:
(i) An isolated phosphorylation site-specific antibody that specifically binds a lipid kinase selected from Column A, Rows 292-298, of Table 1 only when phosphorylated at the tyrosine listed in corresponding Column D, Rows 292-298, of Table 1, comprised within the phosphorylatable peptide sequence listed in corresponding Column E, Rows 292-298, of Table 1 (SEQ ID NOs: 291-297), wherein said antibody does not bind said protein when not phosphorylated at said tyrosine.
(ii) An equivalent antibody to (i) above that only binds the lipid kinase when not phosphorylated at the disclosed site (and does not bind the protein when it is phosphorylated at the site).
(iii) A heavy-isotope labeled peptide (AQUA peptide) for the quantification of a Leukemia-related signaling protein that is an lipid kinase selected from Column A, Rows 292-298, said labeled peptide comprising the phosphorylatable peptide sequence listed in corresponding Column E, Rows 292-298, of Table 1 (SEQ ID NOs: 291-297), which sequence comprises the phosphorylatable tyrosine listed in corresponding Column D, Rows 292-298, of Table 1.

Among this preferred subset of reagents, antibodies and AQUA peptides for the detection/quantification of the following lipid kinase phosphorylation sites are particularly preferred: PIK3CB (Y962) (see SEQ ID NO: 293).

In yet a further subset of preferred embodiments, there is provided:
(i) An isolated phosphorylation site-specific antibody that specifically binds a protein selected from the group consisting of EIF4EBP2, EIF4G2 and EIF4B (Column A, Rows 446, 448 and 460 of Table 1) only when phosphorylated at the tyrosine listed in corresponding Column D, Rows 191, 199, 446, 448 and 460 of Table 1), said tyrosine comprised within the phosphorylatable peptide sequence listed in corresponding Column E, Rows 446, 448 and 460, of Table 1 (SEQ ID NOs: 445, 447 and 459), wherein said antibody does not bind said protein when not phosphorylated at said tyrosine.
(ii) An equivalent antibody to (i) above that only binds the EIF4EBP2, EIF4G2 and EIF4B protein when not phosphorylated at the disclosed site (and does not bind the protein when it is phosphorylated at the site).
(iii) A heavy-isotope labeled peptide (AQUA peptide) for the quantification of a protein selected from the group consisting of EIF4EBP2, EIF4G2 and EIF4B (Column A, Rows 446, 448 and 460 of Table 1), said labeled peptide comprising the phosphorylatable peptide sequence listed in corresponding Column E, Rows 446, 448 and 460, of Table 1 (SEQ ID NOs: 445, 447 and 459), which sequence comprises the phosphorylatable tyrosine listed in corresponding Column D, Rows 446, 448 and 460, of Table 1.

The invention also provides, in part, an immortalized cell line producing an antibody of the invention, for example, a cell line producing an antibody within any of the foregoing preferred subsets of antibodies. In one preferred embodiment, the immortalized cell line is a rabbit hybridoma or a mouse hybridoma.

In certain other preferred embodiments, a heavy-isotope labeled peptide (AQUA peptide) of the invention (for example, an AQUA peptide within any of the foregoing preferred subsets of AQUA peptides) comprises a disclosed site sequence wherein the phosphorylatable tyrosine is phosphorylated. In certain other preferred embodiments, a heavy-isotope labeled peptide of the invention comprises a disclosed site sequence wherein the phosphorylatable tyrosine is not phosphorylated.

The foregoing subsets of preferred reagents of the invention should not be construed as limiting the scope of the invention, which, as noted above, includes reagents for the detection and/or quantification of disclosed phosphorylation sites on any of the other protein type/group subsets (each a preferred subset) listed in Column C of Table 1/FIG. 2.

Also provided by the invention are methods for detecting or quantifying a Leukemia-related signaling protein that is tyrosine phosphorylated, said method comprising the step of utilizing one or more of the above-described reagents of the invention to detect or quantify one or more Leukemia-related signaling protein(s) selected from Column A of Table 1 only when phosphorylated at the tyrosine listed in corresponding Column D of Table 1. In certain preferred embodiments of the methods of the invention, the reagents comprise a subset of preferred reagents as described above.

Also provided by the invention is a method for obtaining a phosphorylation profile of protein kinases that are phosphorylated in Leukemia signaling pathways, said method comprising the step of utilizing one or more isolated antibody that specifically binds a protein inase selected from Column A, Rows 210-291, of Table 1 only when phosphorylated at the tyrosine listed in corresponding Column D, Rows 210-291, of Table 1, comprised within the phosphorylation site sequence listed in corresponding Column E, Rows 210-291, of Table 1 (SEQ ID NOs: SEQ ID NOs: 210-221, 223-280, and 281-290), to detect the phosphorylation of one or more of said protein kinases, thereby obtaining a phosphorylation profile for said kinases.

The identification of the disclosed novel Leukemia-related signaling protein phosphorylation sites, and the standard production and use of the reagents provided by the invention are described in further detail below and in the Examples that follow.

All cited references are hereby incorporated herein, in their entirety, by reference. The Examples are provided to further illustrate the invention, and do not in any way limit its scope, except as provided in the claims appended hereto.

TABLE 1
Newly Discovered Leukemia-related Phosphorylation Sites.

| | A | | | | | |
|---|---|---|---|---|---|---|
| | Protein | | | D | E | |
| | Name | B | C | Phospho- | Phosphorylation | H |
| 1 | (short) | Accession No. | Protein Type | Residue | Site Sequence | SEQ ID NO |
| 2 | ZDHHC17 | NP_056151.2 | Acetyltransferase | Y336 | GLMyGGVWATVQFLSKSFFDHSMH | SEQ ID NO: 1 |
| | | | | | SALPLGIYLATK | |
| 3 | ZDHHC17 | NP_056151.2 | Acetyltransferase | Y364 | GLMYGGVWATVQFLSKSFFDHSMH | SEQ ID NO: 2 |
| | | | | | SALPLGlyLATK | |
| 4 | CNN2 | NP_004359.1 | Actin binding protein | Y184 | CASQSGMTAyGTRR | SEQ ID NO: 3 |
| 5 | DBN1 | NP_004386.1 | Actin binding protein | Y597 | EGTQASEGyFSQSQEEEFAQSEEL | SEQ ID NO: 4 |
| | | | | | CAK | |
| 6 | DBNL | NP_001014436.1 | Actin binding protein | Y140 | VAKASGANySFHK | SEQ ID NO: 5 |
| 7 | FSCN2 | NP_036550.1 | Actin binding protein | Y352 | yVCMKKNGQLAAISDFVGK | SEQ ID NO: 6 |
| 8 | FLNB | NP_001448.2 | Actin binding protein | Y414 | DIYTAGAGVGDIGVEVEDPQGKNT | SEQ ID NO: 7 |
| | | | | | VELLVEDKGNQVy | |
| 9 | LCP1 | | Actin binding protein | Y28 | VDTDGNGyISFNELN | SEQ ID NO: 8 |
| 10 | PIP | NP_002643.1 | Actin binding protein | Y71 | TyLISSIPLQGAFNYKYTACKCDD | SEQ ID NO: 9 |
| | | | | | NPK | |
| 11 | ABI2 | NP_005750.4 | Adaptor/scaffold | Y192 | HSPyRTLEPVRPPVVPNDYVPSPT | SEQ ID NO: 10 |
| | | | | | R | |
| 12 | AMOTL1 | NP_570899.1 | Adaptor/scaffold | Y191 | STQPQQNNEELPTyEEAK | SEQ ID NO: 11 |
| 13 | ANK1 | NP_000028.3 | Adaptor/scaffold | Y1468 | EGQNANMENLyTALQSIDRGEIVN | SEQ ID NO: 12 |
| | | | | | MLEGSGRQSR | |
| 14 | ARRB2 | NP_004304.1 | Adaptor/scaffold | Y404 | LKGMKDDDyDDQLC | SEQ ID NO: 13 |
| 15 | PIK3AP1 | NP_689522.2 | Adaptor/scaffold | Y594 | DRPQSSIySPFAGMK | SEQ ID NO: 14 |
| 16 | PIK3AP1 | | Adaptor/scaffold | Y694 | AKVEFGVyESGPRKS | SEQ ID NO: 15 |
| 17 | NEDD9 | NP_006394.1 | Adaptor/scaffold | Y172 | yQKDVYDIPPSHTTGQVYDIPPSS | SEQ ID NO: 16 |
| | | | | | AK | |
| 18 | NEDD9 | | Adaptor/scaffold | Y177 | YQKDVyDIPPSHTTQGVYDIPPSS | SEQ ID NO: 17 |
| | | | | | AK | |
| 19 | NEDD9 | | Adaptor/scaffold | Y189 | DVYDIPPSHTTQGVyDIPPSSAK | SEQ ID NO: 18 |
| 20 | CD2AP | NP_036252.1 | Adaptor/scaffold | Y361 | yFSLKPEEKDEK | SEQ ID NO: 19 |
| 21 | DIAPH1 | NP_005210.2 | Adaptor/scaffold | Y415 | NDyEARPQYYK | SEQ ID NO: 20 |
| 22 | DAB2 | NP_001334.1 | Adaptor/scaffold | Y685 | KGEQTSSGTLSAFASyFNSK | SEQ ID NO: 21 |
| 23 | DOK2 | | Adaptor/scaffold | Y139 | CMEENELySSAVTVG | SEQ ID NO: 22 |
| 24 | DOK2 | NP_003965.2 | Adaptor/scaffold | Y330 | VPPQLLADPLyDSIEETLPPRPDH | SEQ ID NO: 23 |
| | | | | | IYDEPEGV | |
| 25 | DOK2 | | Adaptor/scaffold | Y345 | ADPLYDSIEETLPPRPDHIyDEPE | SEQ ID NO: 24 |
| | | | | | GV | |
| 26 | GAB2 | | Adaptor/scaffold | Y438 | AGDNSQSVyIPMSPGAHHFDSLGY | SEQ ID NO: 25 |
| | | | | | PSTTLPVHR | |
| 27 | GAB2 | NP_036428.1 | Adaptor/scaffold | Y525 | ANHTFNSSSSQyCR | SEQ ID NO: 26 |
| 28 | C20orf32 | NP_065089.2 | Adaptor/scaffold | Y312 | LSLPEIPSyGFLVPR | SEQ ID NO: 27 |
| 29 | HCLS1 | NP_005326.1 | Adaptor/scaffold | Y360 | GLQVEEEPVyE | SEQ ID NO: 28 |
| 30 | SLC4A1AP | NP_060628.1 | Adaptor/scaffold | Y773 | SSKYPEDDPDyCVW | SEQ ID NO: 29 |
| 31 | LAT2 | NP_054865.2 | Adaptor/scaffold | Y40 | RSEKIyQQR | SEQ ID NO: 30 |
| 32 | LAT2 | | Adaptor/scaffold | Y58 | SFTGSRTySLVGQAW | SEQ ID NO: 31 |
| 33 | LAT2 | NP_054865.2 | Adaptor/scaffold | Y84 | LLQFyPSLEDPASSR | SEQ ID NO: 32 |
| 34 | PDLIM5 | NP_001011513.1 | Adaptor/scaffold | Y138 | yTEFYHVPTHSDASK | SEQ ID NO: 33 |
| 35 | LIMS1 | NP_004978.2 | Adaptor/scaffold | Y304 | FVEFDMKPVCKKCyEK | SEQ ID NO: 34 |
| 36 | SCAP2 | | Adaptor/scaffold | Y237 | YDERGELyDDVDHPL | SEQ ID NO: 35 |
| 37 | SAMSN1 | NP_071419.3 | Adaptor/scaffold | Y160 | LDDDGPySGPFCGR | SEQ ID NO: 36 |
| 38 | SIT1 | NP_055265.1 | Adaptor/scaffold | Y127 | AAEEVMCyTSLQLRPPQGR | SEQ ID NO: 37 |
| 39 | SIT1 | NP_055265.1 | Adaptor/scaffold | Y169 | SQASGPEPELyASVCAQTR | SEQ ID NO: 38 |
| 40 | STAM | NP_003464.1 | Adaptor/scaffold | Y381 | LMNEDPMySMYAK | SEQ ID NO: 39 |
| 41 | STAM | NP_003464.1 | Adaptor/scaffold | Y384 | LMNEDPMYSMyAK | SEQ ID NO: 40 |
| 42 | SCAP1 | NP_003717.2 | Adaptor/scaffold | Y142 | GLFYyYANEK | SEQ ID NO: 41 |
| 43 | FYB | NP_001456.3 | Adaptor/scaffold | Y826 | YGYVLRSYLADNDGEIYDDIADGC | SEQ ID NO: 42 |
| | | | | | IyDND | |
| 44 | SDCBP | NP_001007068.1 | Adaptor/scaffold | Y91 | PSSINyMVAPVTGNDVGIR | SEQ ID NO: 43 |
| 45 | TRIP6 | NP_003293.2 | Adaptor/scaffold | Y149 | TGSLKPNPASPLPASPyGGPTPAS | SEQ ID NO: 44 |
| | | | | | YTTASTPAGPAFPVQVK | |
| 46 | TRIP6 | NP_003293.2 | Adaptor/scaffold | Y157 | TGSLKPNPASPLPASPYGGPTPAS | SEQ ID NO: 45 |
| | | | | | yTTASTPAGPAFPVQVK | |
| 47 | TJP2 | | Adaptor/scaffold | Y423 | PEERRHQySDYDYHS | SEQ ID NO: 46 |
| 48 | TJP2 | | Adaptor/scaffold | Y428 | HQYSDYDyHSSSEKL | SEQ ID NO: 47 |
| 49 | TRAF4 | NP_004286.2 | Adaptor/scaffold | Y212 | EFVFDTIQSHQyQCPR | SEQ ID NO: 48 |
| 50 | CRKL | NP_005198.1 | Adaptor/scaffold | Y127 | TAEDNLEyVRTLYDF | SEQ ID NO: 49 |
| 51 | ZFYVE9 | NP_015563.2 | Adaptor/scaffold | Y741 | LLyMDRKEARVCVICHSVLMNVAQ | SEQ ID NO: 50 |
| | | | | | PR | |
| 52 | TJP1 | NP_003248.2 | Adaptor/scaffold | Y833 | LSYLSAPGSEYSMySTDSR | SEQ ID NO: 51 |
| 53 | TJP2 | NP_004808.2 | Adaptor/scaffold | Y1178 | GyYGQSAR | SEQ ID NO: 52 |
| 54 | LPXN | | Adaptor/scaffold; | Y62 | PLPAQLVyTTNIQEL | SEQ ID NO: 53 |
| | | | Cytoskeletal protein | | | |
| 55 | LPXN | | Adaptor/scaffold; | Y72 | NIQELNVySEAQEPK | SEQ ID NO: 54 |
| | | | Cytoskeletal protein | | | |
| 56 | LPP | | Adaptor/scaffold; | Y234 | SAQPSPHyMAAPSSG | SEQ ID NO: 55 |
| | | | Cytoskeletal protein | | | |
| 57 | LPP | NP_005569.1 | Adaptor/scaffold; | Y346 | REPGYTPPGAGNQNPPGMyPVTGP | SEQ ID NO: 56 |
| | | | Cytoskeletal protein | | K | |
| 58 | G3BP2 | NP_036429.2 | Adaptor/scaffold; | Y175 | QENANSGyYEAHPV | SEQ ID NO: 57 |
| | | | RNA binding protein | | | |
| 59 | G3BP2 | NP_036429.2 | Adaptor/scaffold; | Y176 | QENANSGYyEAHPV | SEQ ID NO: 58 |
| | | | RNA binding protein | | | |
| 60 | ADAM18 | | Adhesion | Y47 | VSERKMIyIITIDGQ | SEQ ID NO: 59 |
| 61 | ADAM18 | NP_055052.1 | Adhesion | Y197 | ALyDYMGSEMMAVTQK | SEQ ID NO: 60 |
| 62 | MLLT4 | NP_005927.2 | Adhesion | Y202 | LAAEVyKDMPETSFTRTISNPEVV | SEQ ID NO: 61 |
| | | | | | MK | |
| 63 | CSPG3 | NP_004377.1 | Adhesion | Y264 | NPQELYDVYCFARELGGEVFyVGP | SEQ ID NO: 62 |
| | | | | | ARR | |
| 64 | DSC1 | NP_004939.1 | Adhesion | Y34 | VyLRVPSHLQAETLVGKVNLEECL | SEQ ID NO: 63 |
| | | | | | K | |
| 65 | DSCAM | NP_001380.2 | Adhesion | Y468 | ISQMITSEGNVVSyLNISSSQVR | SEQ ID NO: 64 |
| 66 | EDIL3 | NP_005702.3 | Adhesion | Y250 | IGSPEYIKSYKIAySNDGKTWAMY | SEQ ID NO: 65 |
| | | | | | K | |
| 67 | EDIL3 | NP_005702.3 | Adhesion | Y260 | IGSPEYIKSYKIAYSNDGKTWAMy | SEQ ID NO: 66 |
| | | | | | K | |
| 68 | ERBB2IP | NP_001006600.1 | Adhesion | Y977 | GPTSGPQSAPQIYGPPQyNIQYSS | SEQ ID NO: 67 |
| | | | | | SAAVK | |
| 69 | FGL1 | NP_004458.3 | Adhesion | Y80 | RQyADCSEIFNDGYK | SEQ ID NO: 68 |
| 70 | LGALS8 | NP_006490.3 | Adhesion | Y332 | EFKVAVNGVHSLEyKHR | SEQ ID NO: 69 |
| 71 | ICAM2 | NP_00864.1 | Adhesion | Y260 | MGTyGVRAAWRR | SEQ ID NO: 70 |
| 72 | PPFIA1 | NP_003617.1 | Adhesion | Y546 | FPMADGHTDSySTSAVLR | SEQ ID NO: 71 |
| 73 | PECAM1 | NP_000433.3 | Adhesion | Y663 | MSDPNMEANSHyGHNDDVR | SEQ ID NO: 72 |
| 74 | SIGLEC6 | NP_001236.3 | Adhesion | Y426 | SDHPAEAGPISEDEQELHy | SEQ ID NO: 73 |
| 75 | SIGLEC6 | NP_001236.3 | Adhesion | Y446 | VQPQEPKVTDTEySEIK | SEQ ID NO: 74 |
| 76 | SCARF1 | NP_003684.2 | Adhesion | Y692 | TVAEHVEAIEGSVQESSGPVTTIy | SEQ ID NO: 75 |
| | | | | | MLAGKPR | |
| 77 | THBS1 | NP_003237.2 | Adhesion | Y1126 | LSHRPKTGFIRVVMyEGK | SEQ ID NO: 76 |
| 78 | URP2 | NP_113659.3 | Adhesion | Y500 | TGSGGPGNHPHGPDASAEGLNPyG | SEQ ID NO: 77 |
| | | | | | LVAPR | |
| 79 | ICAM3 | NP_002153.1 | Adhesion; Immunoglobulin | Y527 | EESTyLPLTSMQPTEAMGEEPSRA | SEQ ID NO: 78 |
| | | | superfamily | | E | |
| 80 | DFFA | NP_004392.1 | Apoptosis | Y75 | DGTIVDDDDyFLCLPSNTKFVALA | SEQ ID NO: 79 |
| | | | | | SNE | |
| 81 | PDCD5 | NP_004699.1 | Apoptosis | Y125 | RKVMDSDEDDDy | SEQ ID NO: 80 |
| 82 | HRC | NP_002143.1 | Calcium-binding protein | Y209 | EEEEEEEEEEEEASTEyGHQAHRH | SEQ ID NO: 81 |
| 83 | CDC45L | NP_003495.1 | Cell cycle regulation | Y413 | SNLDKLyHGLELAK | SEQ ID NO: 82 |
| 84 | CLASP1 | NP_056097.1 | Cell cycle regulation | Y697 | LLGSGyGGLTGGSSRGPPVTPSSE | SEQ ID NO: 83 |
| | | | | | K | |
| 85 | SUGT1 | NP_006695.1 | Cell cycle regulation | Y47 | ALEQKPDDAQyYCQR | SEQ ID NO: 84 |
| 86 | SMC4L1 | NP_001002799.1 | Cell cycle regulation | Y150 | IIDKEGDDyEVIPNSNFYVSR | SEQ ID NO: 85 |
| 87 | TSC2 | NP_000539.1 | Cell cycle regulation; Tumor | Y1736 | SNPTDIyPSKWIARLRHIK | SEQ ID NO: 86 |
| | | | suppressor; GTPase activating | | | |
| | | | protein, misc. | | | |
| 88 | CD22 | | Cell surface | Y796 | TGDAESSEMQRPPPDCDDTVTySA | SEQ ID NO: 87 |
| | | | | | LHKR | |
| 89 | LY9 | NP_002339.2 | Cell surface | Y626 | TPVSQKEESSATIyCSIR | SEQ ID NO: 88 |
| 90 | CD72 | NP_001773.1 | Cell surface | Y39 | LGQDPGADDDGEITyENVQVPAVL | SEQ ID NO: 89 |
| | | | | | GVPSSLASSVLGDK | |
| 91 | APLP2 | NP_001633.1 | Cell surface; DNA binding | Y757 | MQNHGYENPTYKyLEQMQI | SEQ ID NO: 90 |
| | | | protein; Receptor, misc. | | | |
| 92 | CD84 | | Cell surface; Immunoglobulin | Y262 | AASKKTIyTYIMASR | SEQ ID NO: 91 |
| | | | superfamily | | | |
| 93 | CD84 | | Cell surface; Immunoglobulin | Y279 | QPAESRIyDEILQSK | SEQ ID NO: 92 |
| | | | superfamily | | | |
| 94 | CD84 | NP_003865.1 | Cell surface; Immunoglobulin | Y299 | VLPSKEEPVNTVySEVQFADKMGK | SEQ ID NO: 93 |
| | | | superfamily | | | |
| 95 | CACNB3 | NP_000716.2 | Channel, calcium | Y429 | HLEEDyADAYQDLYQPHR | SEQ ID NO: 94 |
| 96 | CACNB3 | NP_000716.2 | Channel, calcium | Y433 | HLEEDYADAyQDLYQPHR | SEQ ID NO: 95 |
| 97 | CACNB3 | NP_000716.2 | Channel, calcium | Y437 | HLEEDYADAYQDLyQPHR | SEQ ID NO: 96 |
| 98 | MGC15619 | NP_115745.2 | Channel, cation | Y35 | HFTVVGDDyHAWNINYKK | SEQ ID NO: 97 |
| 99 | MGC15619 | NP_115745.2 | Channel, cation | Y42 | FLRHFTVVGDDYHAWNINyK | SEQ ID NO: 98 |
| 100 | GABRA1 | NP_000797.2 | Channel, chloride | Y53 | ILFRLLDGyDNRLRPGLGER | SEQ ID NO: 99 |
| 101 | GABRA1 | NP_000797.2 | Channel, chloride | Y237 | NQYDLLGQTVDSGIVQSSTGEyVV | SEQ ID NO: 100 |
| | | | | | MTTHFH | |
| 102 | CFTR | NP_000483.3 | Channel, chloride; | Y1307 | NLDPyEQWSDQEIWKVADEVGLR | SEQ ID NO: 101 |
| | | | Transporter, ABC | | | |
| 103 | P2RX7 | NP_002553.2 | Channel, ligand-gated; | Y288 | TTNVSLyPGYNFRYAK | SEQ ID NO: 102 |
| | | | Receptor, misc. | | | |
| 104 | P2RX7 | NP_002553.2 | Channel, ligand-gated; | Y295 | TTNVSLYPGYNFRyAK | SEQ ID NO: 103 |
| | | | Receptor, misc. | | | |
| 105 | GJA5 | NP_005257.2 | Channel, misc. | Y123 | EAERAKEVRGSGSyEYPVAEK | SEQ ID NO: 104 |
| 106 | GJA5 | NP_005257.2 | Channel, misc. | Y125 | EAERAKEVRGSGSYEyPVAEK | SEQ ID NO: 105 |
| 107 | CCT2 | NP_006422.1 | Chaperone | Y297 | FINRQLIyNYPEQLF | SEQ ID NO: 106 |
| 108 | DNAJC13 | NP_056083.2 | Chaperone | Y1024 | MLNSNTESPyLIWNNSTR | SEQ ID NO: 107 |
| 109 | FKBP8 | NP_036313.3 | Chaperone | Y365 | STETALyR | SEQ ID NO: 108 |
| 110 | HSPCA | NP_001017963.1 | Chaperone | Y319 | VILHLKEDQTEyLEER | SEQ ID NO: 109 |
| 111 | HSPCA | NP_001017963.1 | Chaperone | Y614 | NQKHIyYITGE | SEQ ID NO: 110 |
| 112 | MRCL3 | NP_006462.1 | Contractile protein | Y155 | GNFNyIEFTR | SEQ ID NO: 111 |
| 113 | IL32 | NP_001012649.1 | Cytokine | Y62 | TVAAYyEEQHPE | SEQ ID NO: 112 |
| 114 | ACTN4 | NP_004915.2 | Cytoskeletal protein | Y234 | KDDPVTNLNNAFEVAEKyLDIPK | SEQ ID NO: 113 |
| 115 | ADD1 | NP_001110.2 | Cytoskeletal protein | Y35 | YFDRVDENNPEyLRER | SEQ ID NO: 114 |
| 116 | ADD3 | NP_001112.2 | Cytoskeletal protein | Y446 | WLNSPNTyMK | SEQ ID NO: 115 |
| 117 | BICD2 | NP_001003800.1 | Cytoskeletal protein | Y425 | RQTALDNEKDRDSHEDGDYyEVDI | SEQ ID NO: 116 |
| | | | | | | NGPE |
| 118 | KRT6A | NP_005545.1 | Cytoskeletal protein | Y278 | DVDAAyMNKVELQAK | SEQ ID NO: 117 |
| 119 | CLASP2 | NP_055912.1 | Cytoskeletal protein | Y1231 | SRDyNPYNYSDSISPFNK | SEQ ID NO: 118 |
| 120 | CLASP2 | NP_055912.1 | Cytoskeletal protein | Y1236 | SRDYNPYNySDSISPFNK | SEQ ID NO: 119 |
| 121 | CFL1 | | Cytoskeletal protein | Y68 | GQTVDDPyATFVKML | SEQ ID NO: 120 |
| 122 | CORO1A | NP_009005.1 | Cytoskeletal protein | Y180 | TLGPEVHPDTIySVDW | SEQ ID NO: 121 |
| 123 | CORO1A | NP_009005.1 | Cytoskeletal protein | Y364 | KSDLFQEDLyPPTAGPDPALTAEE | SEQ ID NO: 122 |
| | | | | | WLGGR | |
| 124 | JUP | NP_002221.1 | Cytoskeletal protein | Y660 | ISEDKNPDyR | SEQ ID NO: 123 |
| 125 | EMD | NP_000108.1 | Cytoskeletal protein | Y90 | KEDALLYQSKGyNDDYYEESYFTT | SEQ ID NO: 124 |
| | | | | | R | |
| 126 | EMD | | Cytoskeletal protein | Y155 | KDRERPMyGRDSAYQ | SEQ ID NO: 125 |
| 127 | EMD | | Cytoskeletal protein | Y161 | MYGRDSAyQSITHYR | SEQ ID NO: 126 |
| 128 | EMD | | Cytoskeletal protein | Y181 | RSSLDLSyYPTSSST | SEQ ID NO: 127 |
| 129 | ELMO1 | | Cytoskeletal protein | Y720 | EPSNyDFVYDCN | SEQ ID NO: 128 |
| 130 | ELMO1 | NP_055615.8 | Cytoskeletal protein | Y724 | EPSNYDFVyDCN | SEQ ID NO: 129 |
| 131 | EPLIN | NP_057441.1 | Cytoskeletal protein | Y190 | yNVPLNRLKMMFEKGEPTQTK | SEQ ID NO: 130 |
| 132 | EPLIN | NP_057441.1 | Cytoskeletal protein | Y751 | NRyYDEDEDEE | SEQ ID NO: 131 |
| 133 | EPB41L2 | NP_001422.1 | Cytoskeletal protein | Y88 | SyTLVVAK | SEQ ID NO: 132 |
| 134 | EPB41L2 | NP_001422.1 | Cytoskeletal protein | Y773 | VTEGTIREEQEyEEEVEEEPRPAA | SEQ ID NO: 133 |
| | | | | | K | |
| 135 | KRT2A | NP_000414.2 | Cytoskeletal protein | Y463 | LNDLEEALQQAKEDLARLLRDyQE | SEQ ID NO: 134 |
| | | | | | LMNVK | |
| 136 | LMNB1 | NP_005564.1 | Cytoskeletal protein | Y482 | NTSEQDQPMGGWEMIRKIGDTSVS | SEQ ID NO: 135 |
| | | | | | yK | |
| 137 | MAP1A | NP_002364.5 | Cytoskeletal protein | Y1388 | VVEPKDTAIyQKDE | SEQ ID NO: 136 |
| 138 | MAP1A | NP_002364.5 | Cytoskeletal protein | Y1696 | GREDVALEQDTyWRELSCER | SEQ ID NO: 137 |
| 139 | MAP1B | NP_005900.1 | Cytoskeletal protein | Y1870 | TPGDFSyAYQKPEETTR | SEQ ID NO: 138 |
| 140 | MAP1B | NP_005900.1 | Cytoskeletal protein | Y1872 | TPGDFSYAyQKPEETTRSPDEEDY | SEQ ID NO: 139 |
| | | | | | DYESYEK | |
| 141 | MAP1B | NP_005900.1 | Cytoskeletal protein | Y1892 | SPDEEDYDYESyEK | SEQ ID NO: 140 |
| 142 | MAP1B | NP_005900.1 | Cytoskeletal protein | Y1904 | TSDVGGyYYEK | SEQ ID NO: 141 |
| 143 | MAP1B | NP_005900.1 | Cytoskeletal protein | Y1905 | TSDVGGYyYEK | SEQ ID NO: 142 |
| 144 | MAP1B | NP_005900.1 | Cytoskeletal protein | Y1921 | SPSDSGySYETIGK | SEQ ID NO: 143 |
| 145 | MAP1B | NP_005900.1 | Cytoskeletal protein | Y1955 | TPEEGGySYDISEK | SEQ ID NO: 144 |
| 146 | MAP1B | NP_005900.1 | Cytoskeletal protein | Y1957 | TPEEGGYSyDISEK | SEQ ID NO: 145 |
| 147 | MAP1B | NP_005900.1 | Cytoskeletal protein | Y1972 | TTSPPEVSGySYEK | SEQ ID NO: 146 |
| 148 | MAP1B | NP_005900.1 | Cytoskeletal protein | Y1974 | TTSPPEVSGYSyEK | SEQ ID NO: 147 |
| 149 | MAP1B | NP_005900.1 | Cytoskeletal protein | Y1991 | LLDDISNGyDDSEDGGHTLGDPSY | SEQ ID NO: 148 |
| | | | | | SYETTEK | |
| 150 | MAP1B | NP_005900.1 | Cytoskeletal protein | Y2006 | LLDDISNGYDDSEDGGHTLGDPSy | SEQ ID NO: 149 |
| | | | | | SYETTEK | |
| 151 | MAP1B | NP_005900.1 | Cytoskeletal protein | Y2008 | LLDDISNGYDDSEDGGHTLGDPSY | SEQ ID NO: 150 |
| | | | | | SyETTEK | |
| 152 | MAP1B | NP_005900.1 | Cytoskeletal protein | Y2025 | ITSFPESEGYSyETSTK | SEQ ID NO: 151 |
| 153 | MAP2 | | Cytoskeletal protein | Y1685 | yQPKGGQVR | SEQ ID NO: 152 |
| 154 | MAP4 | NP_002366.2 | Cytoskeletal protein | Y1001 | VSySHIQSK | SEQ ID NO: 153 |
| 155 | NEB | NP_004534.1 | Cytoskeletal protein | Y4112 | AYELQSDNVyKADLEWLR | SEQ ID NO: 154 |
| 156 | PLEC1 | NP_000436.2 | Cytoskeletal protein | Y4283 | SRSSSVGSSSSyPISPAVSR | SEQ ID NO: 155 |
| 157 | RSN | NP_002947.1 | Cytoskeletal protein | Y108 | NDGSVAGVRyFQCEPLK | SEQ ID NO: 156 |
| 158 | TAGLN2 | NP_003555.1 | Cytoskeletal protein | Y8 | GPAyGLSR | SEQ ID NO: 157 |
| 159 | SPTBN1 | NP_842565.1 | Cytoskeletal protein | Y17 | TSSISGPLSPAyTGQVPYNYNQLE | SEQ ID NO: 158 |
| | | | | | GR | |
| 160 | TLN1 | NP_006280.2 | Cytoskeletal protein | Y127 | IGITNHDEySLVR | SEQ ID NO: 159 |
| 161 | HRIHFB2 | NP_008963.3 | Cytoskeletal protein | Y553 | FTSGKYQDVyVELSHIK | SEQ ID NO: 160 |
| | 122 | | | | | |
| 162 | TUBA1 | | Cytoskeletal protein | Y210 | DNEAIyDICRRNLDIERPT | SEQ ID NO: 161 |
| 163 | TUBA1 | | Cytoskeletal protein | Y224 | NLDIERPTyTNLNR | SEQ ID NO: 162 |
| 164 | TUBA1 | NP_005991.1 | Cytoskeletal protein | Y282 | AyHEQLSVAEITNACFEPANQMVK | SEQ ID NO: 163 |
| 165 | TUBA1 | NP_005991.1 | Cytoskeletal protein | Y399 | LDHKFDLMyAKR | SEQ ID NO: 164 |
| 166 | TUBA3 | NP_006000.2 | Cytoskeletal protein | Y451 | EDMAALEKDYEEVGVGSVEGEGEE | SEQ ID NO: 165 |
| | | | | | EGEEy | |
| 167 | TUBB2 | NP_001060.1 | Cytoskeletal protein | Y106 | GHyTEGAELVDSVLDVVRK | SEQ ID NO: 166 |
| 168 | TUBB2 | NP_001060.1 | Cytoskeletal protein | Y222 | LTTPTyGDLNHLVSATMSGVTTCL | SEQ ID NO: 167 |
| | | | | | R | |
| 169 | TUBB | NP_821133.1 | Cytoskeletal protein | Y51 | ISVYyNEATGGK | SEQ ID NO: 168 |
| 170 | VIM | NP_003371.2 | Cytoskeletal protein | Y276 | DVRQQyESVAAK | SEQ ID NO: 169 |
| 171 | RAD51 | NP_002866.2 | DNA repair | Y232 | YALLIVDSATALYRTDySGRGELS | SEQ ID NO: 170 |
| | | | | | ARQMHLAR | |
| 172 | XRCC1 | NP_006288.1 | DNA repair | Y576 | RKLIRYVTAFNGELEDyMSDR | SEQ ID NO: 171 |
| 173 | RFC2 | NP_002905.2 | DNA replication | Y277 | TFQMAEyLKLEFIKEIGYTHMK | SEQ ID NO: 172 |
| 174 | GIMAP1 | NP_570115.1 | G protein regulator, misc. | Y14 | MATDEENVyGLEENAQSR | SEQ ID NO: 173 |
| 175 | ARHGDIA | NP_004300.1 | G protein regulator, misc. | Y27 | HSVNyKPPAQKSIQE | SEQ ID NO: 174 |
| 176 | ARHGDIB | NP_001166.3 | G protein regulator, misc. | Y48 | SLKELQEMDKDDESLIKyK | SEQ ID NO: 175 |
| 177 | SIPA1L3 | NP_055888.1 | G protein regulator, misc. | Y1068 | RPVSFPETPyTVSPAGADR | SEQ ID NO: 176 |
| 178 | GNA12 | NP_002061.1 | G protein, heterotrimeric | Y61 | IIHEDGySEEECR | SEQ ID NO: 177 |
| 179 | GNA15 | NP_002059.1 | G protein, heterotrimeric | Y83 | QMRIIHGAGYSEEERKGFRPLVyQ | SEQ ID NO: 178 |
| | | | | | NIFVSMR | |
| 180 | GNA13 | NP_006487.1 | G protein, heterotrimeric | Y354 | NNLKECGLy | SEQ ID NO: 179 |
| 181 | ARFGAP1 | NP_060679.1 | GTPase activating protein, | Y208 | GNTPPPQKKEDDFLNNAMSSLySG | SEQ ID NO: 180 |
| | | | ARF | | W | |
| 182 | ARFGAP3 | | GTPase activating protein, | Y378 | WDDSSDSyWKKETSK | SEQ ID NO: 181 |
| | | | ARF | | | |
| 183 | CENTB1 | NP_055531.1 | GTPase activating protein, | Y712 | EAEAAQGQAGDETyLDIFR | SEQ ID NO: 182 |
| | | | ARF | | | |
| 184 | CENTD3 | NP_071926.4 | GTPase activating protein, | Y303 | LTPLLSGWLDKLSPQGNyVFQR | SEQ ID NO: 183 |
| | | | ARF; GTPase activating | | | |
| | | | protein, Rac/Rho | | | |
| 185 | CENTD3 | NP_071926.4 | GTPase activating protein, | Y139 | SLMyFGSDKDPFPK | SEQ ID NO: 184 |
| | | | ARF; GTPase activating | | | |
| | | | protein, Rac/Rho | | | |
| 186 | CENTD3 | NP_071926.4 | GTPase activating protein, | Y489 | QSWAAALQEAVTETLSDyEVAEK | SEQ ID NO: 185 |
| | | | ARF; GTPase activating | | | |
| | | | protein, Rac/Rho | | | |
| 187 | CENTD3 | NP_071926.4 | GTPase activating protein, | Y684 | ATYSGFLyCSPVSNK | SEQ ID NO: 186 |
| | | | ARF; GTPase activating | | | |
| | | | protein, Rac/Rho | | | |
| 188 | CENTD3 | NP_071926.4 | GTPase activating protein, | Y882 | TLyGQGEGR | SEQ ID NO: 187 |
| | | | ARF; GTPase activating | | | |
| | | | protein, Rac/Rho | | | |
| 189 | SIPA1L1 | NP_056371.1 | GTPase activating protein, | Y1166 | TGSVGGTyRQKSMPE | SEQ ID NO: 188 |
| | | | misc. | | | |
| 190 | ARHGAP4 | NP_001657.2 | GTPase activating protein, | Y483 | GDKEEQEVSWTQyTQRK | SEQ ID NO: 189 |
| | | | Rac/Rho | | | |
| 191 | RGS10 | NP_001005339.1 | GTPase activating protein, | Y94 | EIyMTFLSSKASSQVNVEGQSR | SEQ ID NO: 190 |
| | | | RGS | | | |
| 192 | RGS14 | NP_006471.2 | GTPase activating protein, | Y122 | NIyQEFLSSQALSPVNIDR | SEQ ID NO: 191 |
| | | | RGS | | | |
| 193 | ARL1 | NP_001168.1 | Guanine nucleotide exchange | Y58 | NVETVTyKNLKFQVW | SEQ ID NO: 192 |
| | | | factor, ARF | | | |
| 194 | CENTD2 | NP_056057.1 | Guanine nucleotide exchange | Y191 | ARLSSAyLLGVPGSEQPDRAGSLE | SEQ ID NO: 193 |
| | | | factor, ARF | | LR | |
| 195 | WBSCR16 | | Guanine nucleotide exchange | Y216 | KVVENEIySESHRVH | SEQ ID NO: 194 |
| | | | factor, misc. | | | |
| 196 | ARHGEF6 | NP_004831.1 | Guanine nucleotide exchange | Y644 | KPSEEEyVIRK | SEQ ID NO: 195 |
| | | | factor, Rac/Rho | | | |
| 197 | RAC2 | NP_002863.1 | Guanine nucleotide exchange | Y139 | EKKLAPITyPQGLALAKEIDSVK | SEQ ID NO: 196 |
| | | | factor, Rac/Rho | | | |
| 198 | ARHGEF2 | NP_004714.2 | Guanine nucleotide exchange | Y866 | SLPAGDALyLSFNPPQPSR | SEQ ID NO: 197 |
| | | | factor, Rac/Rho | | | |
| 199 | VAV1 | | Guanine nucleotide exchange | Y826 | GWWRGEIyGRVGWFP | SEQ ID NO: 198 |
| | | | factor, Rac/Rho | | | |
| 200 | RAPGEF1 | | Guanine nucleotide exchange | Y341 | RLSGGSHSyGGESPRLSPCSSIDK | SEQ ID NO: 199 |
| | | | factor, Ras | | LSK | |
| 201 | DDX46 | NP_055644.2 | Helicase | Y730 | yAGDIIKALELSGTAVPPDLEK | SEQ ID NO: 200 |
| 202 | DDX3X | NP_001347.3 | Helicase | Y580 | QEVPSWLENMAYEHHyK | SEQ ID NO: 201 |
| 203 | DDX20 | NP_009135.3 | Helicase | Y756 | LQTEAQEDDWyDCHR | SEQ ID NO: 202 |
| 204 | BAT1 | NP_004631.1 | Helicase | Y39 | GSyVSIHSSGFR | SEQ ID NO: 203 |
| 205 | WRN | NP_000544.2 | Helicase | Y212 | LyAATDAYAGFIIYR | SEQ ID NO: 204 |
| 206 | CD7 | NP_006128.1 | Immunoglobulin superfamily | Y239 | CNTLSSPNQyQ | SEQ ID NO: 205 |
| 207 | SLAMF9 | NP_254273.1 | Immunoglobulin superfamily | Y125 | TSQISTMQQyNLCVYR | SEQ ID NO: 206 |
| 208 | ANP32A | NP_006296.1 | Inhibitor protein | Y148 | LLPQLTYLDGyDR | SEQ ID NO: 207 |
| 209 | SERPINC1 | NP_000479.1 | Inhibitor protein | Y163 | TSDQIHFFFAKLNCRLyR | SEQ ID NO: 208 |
| 210 | AK2 | | Kinase (non-protein) | Y200 | IRLQAYHTQTTPLIEyYRK | SEQ ID NO: 209 |
| 211 | CMPK | NP_057392.1 | Kinase (non-protein) | Y187 | RIQTyLQSTKPIIDLYEEMGKVKK | SEQ ID NO: 210 |
| | | | | | IDASK | |
| 212 | CMPK | NP_057392.1 | Kinase (non-protein) | Y198 | RIQTYLQSTKPIIDLyEEMGKVKK | SEQ ID NO: 212 |
| | | | | | IDASK | |
| 213 | HK1 | NP_000179.1 | Kinase (non-protein) | Y749 | MISGMyLGEIVR | SEQ ID NO: 212 |
| 214 | PRPS1 | NP_002755.1 | Kinase (non-protein) | Y146 | QGFFDIPVDNLyAEPA | SEQ ID NO: 213 |
| 215 | TRIM24 | NP_003843.3 | KINASE(atypical); Protein | Y506 | yPPNQNIPRQAIKPNPLQMAFLAQ | SEQ ID NO: 214 |
| | | | kinase | | QAIK | |
| 216 | TRIM28 | NP_005753.1 | KINASE(atypical); Protein | Y242 | DCQLNAHKDHQyQFLEDAVR | SEQ ID NO: 215 |
| | | | kinase | | | |
| 217 | TRIM28 | NP_005753.1 | KINASE(atypical); Protein | Y369 | LIyFQLHR | SEQ ID NO: 216 |
| | | | kinase | | | |
| 218 | TRIM33 | NP_056990.3 | KINASE(atypical); Protein | Y1018 | HSQHyQIPDDFVADVRLIFK | SEQ ID NO: 217 |
| | | | kinase | | | |
| 219 | PTK9 | NP_002813.2 | KINASE(dual); Protein kinase, | Y135 | QKMLyAATRATLKKEFGGGHIK | SEQ ID NO: 218 |
| | | | dual-specificity | | | |
| 220 | DYRK1A | NP_001387.2 | KINASE(dual); Protein kinase, | Y177 | YEIDSLIGKGSFGQVVKAyDR | SEQ ID NO: 219 |
| | | | dual-specificity | | | |
| 221 | NPR2 | NP_003986.2 | KINASE(dual); Protein kinase, | Y725 | SGPFyLEGLDLSPKEIVQK | SEQ ID NO: 220 |
| | | | dual-specificity | | | |
| 222 | TTK | NP_003309.2 | KINASE(dual); Protein kinase, | Y811 | GTTEEMKyVLGQLVGLNSPNSILK | SEQ ID NO: 221 |
| | | | dual-specificity | | | |
| 223 | PKIA | | KINASE(regulator); Protein | Y8 | MTDVETTyADFIASGRTGR | SEQ ID NO: 222 |
| | | | kinase, regulatory subunit | | | |
| 224 | PRKAR1B | NP_002726.1 | KINASE(regulator); Protein | Y312 | SPNEEyVEVGR | SEQ ID NO: 223 |
| | | | kinase, regulatory subunit | | | |
| 225 | BCR | NP_004318.3 | KINASE(S/T); GTPase | Y58 | MIyLQTLLAK | SEQ ID NO: 224 |
| | | | activating protein, Rac/Rho; | | | |
| | | | Protein kinase, Ser/Thr | | | |
| | | | (non-receptor) | | | |
| 226 | BCR | NP_004318.3 | KINASE(S/T); GTPase | Y231 | SQHGAGSSVGDASRPPyR | SEQ ID NO: 225 |
| | | | activating protein, Rac/Rho; | | | |
| | | | Protein kinase, Ser/Thr | | | |
| | | | (non-receptor) | | | |
| 227 | BCR | NP_004318.3 | KINASE(S/T); GTPase | Y554 | VPELyEIHKEFYDGLFPR | SEQ ID NO: 226 |
| | | | activating protein, Rac/Rho; | | | |
| | | | Protein kinase, Ser/Thr | | | |
| | | | (non-receptor) | | | |
| 228 | BCR | NP_004318.3 | KINASE(S/T); GTPase | Y561 | VPELYEIHKEFyDGLFPR | SEQ ID NO: 227 |
| | | | activating protein, Rac/Rho; | | | |
| | | | Protein kinase, Ser/Thr | | | |
| | | | (non-receptor) | | | |
| 229 | BCR | NP_004318.3 | KINASE(S/T); GTPase | Y852 | SYTFLISSDyER | SEQ ID NO: 228 |
| | | | activating protein, Rac/Rho; | | | |
| | | | Protein kinase, Ser/Thr | | | |
| | | | (non-receptor) | | | |
| 230 | ATM | NP_000042.3 | KINASE(S/T); Kinase, lipid; | Y2129 | EVEGTSYHESLyNALQSLRDREFS | SEQ ID NO: 229 |
| | | | Protein kinase, Ser/Thr | | TFYESLKYAR | |
| | | | (non-receptor) | | | |
| 231 | RIOK2 | NP_060813.1 | KINASE(S/T); Protein kinase | Y445 | VQGGVPAGSDEyEDECPHLIALSS | SEQ ID NO: 230 |
| | | | | | LNR | |
| 232 | RIOK2 | NP_060813.1 | KINASE(S/T); Protein kinase | Y477 | EFRPFRDEENVGAMNQyR | SEQ ID NO: 231 |
| 233 | RIPK4 | NP_065690.2 | KINASE(S/T); Protein kinase, | Y576 | GVDVSLQGKDAWLPLHyAAWQGHL | SEQ ID NO: 232 |
| | | | Ser/Thr (non-receptor) | | PIVKLLAK | |
| 234 | STK6 | NP_003591.2 | KINASE(S/T); Protein kinase, | Y236 | LSKFDEQRTATyITELANALSYCH | SEQ ID NO: 233 |
| | | | Ser/Thr (non-receptor) | | SK | |
| 235 | STK6 | NP_003591.2 | KINASE(S/T); Protein kinase, | Y246 | LSKFDEQRTATITELANALSyCHS | SEQ ID NO: 234 |
| | | | Ser/Thr (non-receptor) | | K | |
| 236 | CSNK1A1 | NP_001883.4 | KINASE(S/T); Protein kinase, | Y294 | TLNHQYDyTFDWTMLK | SEQ ID NO: 235 |
| | | | Ser/Thr (non-receptor) | | | |
| 237 | CDC2L5 | NP_003709.2 | KINASE(S/T); Protein kinase, | Y300 | EPPKAyREDK | SEQ ID NO: 236 |
| | | | Ser/Thr (non-receptor) | | | |
| 238 | CDKL2 | NP_003939.1 | KINASE(S/T); Protein kinase, | Y15 | YENLGLVGEGSyGMVMKCR | SEQ ID NO: 237 |
| | | | Ser/Thr (non-receptor) | | | |
| 239 | GRK4 | NP_001004056.1 | KINASE(S/T); Protein kinase, | Y222 | FVVSLAYAyETK | SEQ ID NO: 238 |
| | | | Ser/Thr (non-receptor) | | | |
| 240 | LATS1 | NP_004681.1 | KINASE(S/T); Protein kinase, | Y779 | DNLyFVMDYIPGGDMMSLLIR | SEQ ID NO: 239 |
| | | | Ser/Thr (non-receptor) | | | |
| 241 | MARK2 | NP_004945.3 | KINASE(S/T); Protein kinase, | Y389 | FSDQAGPAIPTSNSySKK | SEQ ID NO: 240 |
| | | | Ser/Thr (non-receptor) | | | |
| 241 | MARK2 | NP_004945.3 | KINASE(S/T); Protein kinase, | Y389 | FSDQAGPAIPTSNSySKK | SEQ ID NO: 240 |
| | | | Ser/Thr (non-receptor) | | | |
| 242 | MARK2 | NP_004945.3 | KINASE(S/T); Protein kinase, | Y525 | DQQNLPyGVTPASPSGHSQGR | SEQ ID NO: 241 |
| | | | Ser/Thr (non-receptor) | | | |
| 243 | MARK3 | NP_002367.4 | KINASE(S/T); Protein kinase, | Y432 | RYSDHAGPAIPSVVAyPK | SEQ ID NO: 242 |
| | | | Ser/Thr (non-receptor) | | | |
| 244 | MELK | NP_055606.1 | KINASE(S/T); Protein kinase, | Y438 | SAVKNEEyFMFPEPK | SEQ ID NO: 243 |
| | | | Ser/Thr (non-receptor) | | | |
| 245 | MAP3K3 | NP_002392.2 | KINASE(S/T); Protein kinase, | Y155 | ASQSAGDINTIyQPPEPR | SEQ ID NO: 244 |
| | | | Ser/Thr (non-receptor) | | | |
| 246 | MINK1 | NP_722549.2 | KINASE(S/T); Protein kinase, | Y706 | SNSAWQIyLQR | SEQ ID NO: 245 |
| | | | Ser/Thr (non-receptor) | | | |
| 247 | MINK1 | NP_722549.2 | KINASE(S/T); Protein kinase, | Y900 | GQSPPSKDGSGDyQSR | SEQ ID NO: 246 |
| | | | Ser/Thr (non-receptor) | | | |
| 248 | MYLK | NP_444253.2 | KINASE(S/T); Protein kinase, | Y464 | QEGSIEVyEDAGSHYLCLLK | SEQ ID NO: 247 |
| | | | Ser/Thr (non-receptor) | | | |
| 249 | MYLK | NP_444253.2 | KINASE(S/T); Protein kinase, | Y471 | QEGSIEVYEDAGSHyLCLLK | SEQ ID NO: 248 |
| | | | Ser/Thr (non-receptor) | | | |
| 250 | MAPK14 | NP_001306.1 | KINASE(S/T); Protein kinase, | Y24 | yQNLSPVGSGAYGSVCAAFDTKTG | SEQ ID NO: 249 |
| | | | Ser/Thr (non-receptor) | | LR | |
| 251 | ALS2CR7 | NP_631897.1 | KINASE(S/T); Protein kinase, | Y63 | LGEGSyATVYKGISRINGQLVALK | SEQ ID NO: 250 |
| | | | Ser/Thr (non-receptor) | | | |
| 252 | PRKCZ | NP_002735.3 | KINASE(S/T); Protein kinase, | Y356 | FyAAEICIALNFLHER | SEQ ID NO: 251 |
| | | | Ser/Thr (non-receptor) | | | |
| 253 | MARK3 | | KINASE(S/T); Protein kinase, | Y402 | ySDHAGPGIPSVVAYPKRSQTSTA | SEQ ID NO: 252 |
| | | | Ser/Thr (non-receptor) | | DSDLK | |
| 254 | STK31 | NP_113602.2 | KINASE(S/T); Protein kinase, | Y992 | YTLyKKEEE | SEQ ID NO: 253 |
| | | | Ser/Thr (non-receptor) | | | |
| 255 | DKFZp76 | XP_291277.2 | KINASE(S/T); Protein kinase, | Y132 | QEDAPVVyLGSFR | SEQ ID NO: 254 |
| | 1P0423 | | Ser/Thr (non-receptor) | | | |
| 256 | STK39 | NP_037365.1 | KINASE(S/T); Protein kinase, | Y446 | QIQSLSVHDSQGPPNANEDyRE | SEQ ID NO: 255 |
| | | | Ser/Thr (non-receptor) | | | |
| 257 | TSSK1 | NP_114417.1 | KINASE(S/T); Protein kinase, | Y12 | RGyLLGINLGEGSYAKVK | SEQ ID NO: 256 |
| | | | Ser/Thr (non-receptor) | | | |
| 258 | TTN | NP_003310.3 | KINASE(S/T); Protein kinase, | Y1845 | SKRFRVRyDGIHYLDIVDCKSYDT | SEQ ID NO: 257 |
| | | | Ser/Thr (non-receptor) | | GEVK | |
| 259 | TTN | NP_003310.3 | KINASE(S/T); Protein kinase, | Y1850 | SKRFRVRYDGIHyLDIVDCKSYDT | SEQ ID NO: 258 |
| | | | Ser/Thr (non-receptor) | | GEVK | |
| 260 | TTN | NP_003310.3 | KINASE(S/T); Protein kinase, | Y1859 | SKRFRVRYDGIHYLDIVDCKSyDT | SEQ ID NO: 259 |
| | | | Ser/Thr (non-receptor) | | GEVK | |
| 261 | TTN | NP_003310.3 | KINASE(S/T); Protein kinase, | Y8052 | DLIONGEyFFR | SEQ ID NO: 260 |
| | | | Ser/Thr (non-receptor) | | | |
| 262 | KALRN | NP_003938.1 | KINASE(S/T); Protein kinase, | Y1351 | yEQLPEDVGHCFVTWADKFQMYVT | SEQ ID NO: 261 |
| | | | Ser/Thr (non-receptor) | | YCKNK | |
| 263 | KALRN | NP_003938.1 | KINASE(S/T); Protein kinase, | Y1372 | YEQLPEDVGHVFVTWADKFQMyVT | SEQ ID NO: 262 |
| | | | Ser/Thr (non-receptor) | | YCKNK | |
| 264 | KALRN | NP_003938.1 | KINASE(S/T); Protein kinase, | Y1375 | YEQLPEDVGHCFVTWADKFQMYVT | SEQ ID NO: 263 |
| | | | Ser/Thr (non-receptor) | | yCKNK | |
| 265 | AKT3 | NP_005456.1 | KINASE(S/T); Protein kinase, | Y251 | TRFyGAEIVSALDYLHSGKIVYR | SEQ ID NO: 264 |
| | | | Ser/Thr (non-receptor) | | | |
| 266 | AKT3 | NP_005456.1 | KINASE(S/T); Protein kinase, | Y269 | TRFYGAEIVSALDYLHSGKIVyR | SEQ ID NO: 265 |
| | | | Ser/Thr (non-receptor) | | | |
| 267 | WNK1 | NP_061852.1 | KINASE(S/T); Protein kinase, | Y2276 | GHMNyEGPGMAR | SEQ ID NO: 266 |
| | | | Ser/Thr (non-receptor) | | | |
| 268 | MAPK3 | NP_002737.2 | KINASE(S/T); Protein kinase, | Y210 | IADPEHDHTGFLTEYVATRWyR | SEQ ID NO: 267 |
| | | | Ser/Thr (non-receptor) | | | |
| | | | Transcription factor | | | |
| 269 | ACVR2B | NP_001097.1 | KINASE(S/T); Receptor | Y85 | RLHCYASWANSSGTIELVKKGCWL | SEQ ID NO: 268 |
| | | | Ser/Thr kinase | | DDFNCyDR | |
| 270 | BMPR1A | NP_004320.2 | KINASE(S/T); Receptor | Y407 | RyMAPEVLDESLNK | SEQ ID NO: 269 |
| | | | Ser/Thr kinase | | | |
| 271 | BLK | NP_001706.2 | KINASE(Y); Protein kinase, | Y187 | CLDEGGyYISPR | SEQ ID NO: 270 |
| | | | tyrosine (non-receptor) | | | |
| 272 | BTK | NP_000052.1 | KINASE(Y); Protein kinase, | Y344 | HYVVCSTPQSQyYLAEK | SEQ ID NO: 271 |
| | | | tyrosine (non-receptor) | | | |
| 273 | LCK | NP_005347.2 | KINASE(Y); Protein kinase, | Y470 | MVRPDNCPEELyQLMR | SEQ ID NO: 272 |
| | | | tyrosine (non-receptor) | | | |
| 274 | SYK | NP_003168.2 | KINASE(Y); Protein kinase, | Y296 | IKSySFPKPGHR | SEQ ID NO: 273 |
| | | | tyrosine (non-receptor) | | | |
| 275 | SYK | NP_003168.2 | KINASE(Y); Protein kinase, | Y630 | LRNYyYDVVN | SEQ ID NO: 274 |
| | | | tyrosine (non-receptor) | | | |
| 276 | SYK | NP_003168.2 | KINASE(Y); Protein kinase, | Y631 | LRNYYyDVVN | SEQ ID NO: 275 |
| | | | tyrosine (non-receptor) | | | |
| 277 | TEC | NP_003206.1 | KINASE(Y); Protein kinase, | Y519 | RYFLDDQyTSSSGAK | SEQ ID NO: 276 |
| | | | tyrosine (non-receptor) | | | |
| 278 | TYK2 | NP_003322.2 | KINASE(Y); Protein kinase, | Y433 | LTADSSHyLCHEVAPPR | SEQ ID NO: 277 |
| | | | tyrosine (non-receptor) | | | |
| 279 | TYK2 | NP_003322.2 | KINASE(Y); Protein kinase, | Y914 | VSLyCYDPTNDGTGEMVAVK | SEQ ID NO: 278 |
| | | | tyrosine (non-receptor) | | | |
| 280 | ABL1 | NP_005148.2 | KINASE(Y); Protein kinase, | Y128 | HSWyHGPVSR | SEQ ID NO: 279 |
| | | | tyrosine (non-receptor) | | | |
| 281 | FES | | KINASE(Y); Protein kinase, | Y713 | EEADGVyAASGGLR | SEQ ID NO: 280 |
| | | | tyrosine (non-receptor) | | | |
| 282 | LYN | NP_002341.1 | KINASE(Y); Protein kinase, | Y266 | LGAGQFGEVWMGYyNNSTK | SEQ ID NO: 281 |
| | | | tyrosine (non-receptor) | | | |
| 283 | ZAP70 | NP_001070.2 | KINASE(Y); Protein kinase, | Y69 | QLNGTyAIAGGK | SEQ ID NO: 282 |
| | | | tyrosine (non-receptor) | | | |
| 284 | ZAP70 | NP_001070.2 | KINASE(Y); Protein kinase, | Y164 | MPWyHSSLTR | SEQ ID NO: 283 |
| | | | tyrosine (non-receptor) | | | |
| 285 | EPHA7 | NP_004431.1 | KINASE(Y); Receptor tyrosine | Y511 | STSASINNLKPGTVyVFQIR | SEQ ID NO: 284 |
| | | | kinase, | | | |
| 286 | FLT3 | NP_004110.1 | KINASE(Y); Receptor tyrosine | Y630 | VMNATAyGISK | SEQ ID NO: 285 |
| | | | kinase, | | | |
| 287 | FLT3 | NP_004110.1 | KINASE(Y); Receptor tyrosine | Y726 | TWTEIFKEHNFSFyPTFQSHPNSS | SEQ ID NO: 286 |
| | | | kinase, | | MPGSR | |
| 288 | FLT3 | NP_004110.1 | KINASE(Y); Receptor tyrosine | Y768 | EVQIHPDSDQISGLHGNSFHSEDE | SEQ ID NO: 287 |
| | | | kinase, | | IEyENQK | |
| 289 | ROS1 | NP_002935.2 | KINASE(Y); Receptor tyrosine | Y363 | KAANMSDVSDLRIFyR | SEQ ID NO: 288 |
| | | | kinase, | | | |
| 290 | TEK | NP_000450.2 | KINASE(Y); Receptor tyrosine | Y897 | NLLGACEHRGy | SEQ ID NO: 289 |
| | | | kinase, | | | |
| 291 | TYRO3 | NP_006284.2 | KINASE(Y); Receptor tyrosine | Y681 | KIySGDYYR | SEQ ID NO: 290 |
| | | | kinase, | | | |
| 292 | DGKA | NP_001336.2 | Kinase, lipid | Y623 | RPHGDIyGINQALGATAK | SEQ ID NO: 291 |
| 293 | PIK4CA | NP_002641.1 | Kinase, lipid | Y284 | yISLSEK | SEQ ID NO: 292 |
| 294 | PIK3CB | NP_006210.1 | Kinase, lipid | Y962 | ERVPFILTyDFIHVIQQGK | SEQ ID NO: 293 |
| 295 | PIK3CB | NP_006210.1 | Kinase, lipid | Y1023 | RHGNLFITLFALMLTAGLPELTSV | SEQ ID NO: 294 |
| | | | | | KDIQyLK | |
| 296 | PIK3CD | | Kinase, lipid | Y484 | EVAPHPVyYPALEKI | SEQ ID NO: 295 |
| 297 | PIK3C2B | NP_002637.2 | Kinase, lipid | Y68 | QNADPSLISWDEPGVDFySKPAGR | SEQ ID NO: 296 |
| 298 | PIK3R1 | NP_852556.2 | Kinase, lipid | Y286 | QAAEyREIDKR | SEQ ID NOl 297 |
| 299 | EPRS | | Ligase | Y690 | GFFICDQPYEPVSPySCK | SEQ ID NOl 298 |
| 300 | PAICS | NP_006443.1 | Ligase | Y22 | EVyELLDSPGK | SEQ ID NO: 299 |
| 301 | OSBP | NP_002547.1 | Lipid binding protein | Y119 | GYQRRWFVLSNGLLSyYRSKAEMR | SEQ ID NO: 300 |
| 302 | OSBP | NP_002547.1 | Lipid binding protein | Y120 | GYQRRWFVLSNGLLSYyRSKAEMR | SEQ ID NO: 301 |
| 303 | OSBP | NP_002547.1 | Lipid binding protein | Y767 | EAEAMKATEDGTPYDPyKALWFER | SEQ ID NO: 302 |
| 304 | DNMT1 | NP_001370.1 | Methyltransferase | Y399 | LSIFDANESGFESyEALPQHK | SEQ ID NO: 303 |
| 305 | DNMT1 | NP_001370.1 | Methyltransferase | Y969 | KEPVDEDLyPEHYRK | SEQ ID NO: 304 |
| 306 | KIAA0339 | NP_055527.1 | Methyltransferase | Y748 | EAyHLPMPMAAEPLPSSSVSGEEA | SEQ ID NO: 305 |
| | | | | | RLPPR | |
| 307 | DNCI2 | NP_001369.1 | Motor protein | Y327 | TTPEyVFHCQSAVMSATFAK | SEQ ID NO: 306 |
| 308 | KNS2 | NP_005543.2 | Motor protein | Y449 | DGTSFGEyGGWYK | SEQ ID NO: 307 |
| 309 | KIF20A | NP_005724.1 | Motor protein | Y869 | TPTCQSSTDCSPyAR | SEQ ID NO: 308 |
| 310 | KLC2 | NP_073733.1 | Motor protein | Y434 | DSAPYGEyGSWYK | SEQ ID NO: 309 |
| 311 | MYO1E | NP_004989.2 | Motor protein | Y950 | NTTQNTGYSSGTQNANyPVR | SEQ ID NO: 310 |
| 312 | MYO9B | NP_004136.2 | Motor protein | Y1683 | IQSHCSyTYGR | SEQ ID NO: 311 |
| 313 | NYO7A | NP_000251.1 | Motor protein | Y142 | KIGEMPPHIFAIADNCyFNMKR | SEQ ID NO: 312 |
| 314 | NY07A | NP_000251.1 | Motor protein | Y1211 | FVKyLRNFIHGGPPGYAPYCEER | SEQ ID NO: 313 |
| 315 | MYH10 | NP_005955.1 | Motor protein | Y22 | AVIyNPATQADWTAK | SEQ ID NO: 314 |
| 316 | MYH9 | NP_002464.1 | Motor protein | Y190 | VIQyLAYVASSHK | SEQ ID NO: 315 |
| 317 | MYH9 | NP_002464.1 | Motor protein | Y193 | KVIQYLAyVASSHK | SEQ ID NO: 316 |
| 318 | SEC24C | | Motor protein | Y296 | ARGPQSNyGGPYPAA | SEQ ID NO: 317 |
| 319 | SEC24C | | Motor protein | Y300 | QSNYGGPyPAAPTFG | SEQ ID NO: 318 |
| 320 | TNNT2 | NP_000355.2 | Motor protein | Y266 | yEINVLRNRINDNQKVSKTRGKAK | SEQ ID NO: 319 |
| | | | | | VTGRWK | |
| 321 | TUBA6 | NP_116093.1 | Motor protein | Y432 | ALEKDyEEVGADSADGEDEGEE | SEQ ID NO: 320 |
| 322 | ALDH9A1 | NP_000687.2 | Oxidoreductase | Y476 | VTIEyYSQLK | SEQ ID NO: 321 |
| 323 | AKR7A2 | NP_003680.2 | Oxidoreductase | Y223 | FyAYNPLAGGLLTGKYKYEDK | SEQ ID NO: 322 |
| 324 | AKR7A2 | NP_003680.2 | Oxidoreductase | Y225 | LGCQDAFPEVyDK | SEQ ID NO: 323 |
| 325 | DHCR24 | NP_055577.1 | Oxidoreductase | Y507 | LGCQDAFPEVyDK | SEQ ID NO: 324 |
| 326 | GPD1 | NP_005267.2 | Oxidoreductase | Y326 | LQGPETARELYSILQHKGLVDKFP | SEQ ID NO: 325 |
| | | | | | LFMAVyK | |
| 327 | HSD17B2 | NP_002144.1 | Oxidoreductase | Y232 | GRLVNVSSMGGGAPMERLASyGSS | SEQ ID NO: 326 |
| | | | | | K | |
| 328 | IDH2 | NP_002159.2 | Oxidoreductase | Y258 | AyDGRFKDIFQEIFDK | SEQ ID NO: 327 |
| 329 | NDUFS7 | NP_077718.2 | Oxidoreductase | Y146 | yVVSMGSCANGGGYYHYSYSVVR | SEQ ID NO: 328 |
| 330 | NDUFS7 | NP_077718.2 | Oxidoreductase | Y162 | YVVSMGSCANGGGYYHySYSVVR | SEQ ID NO: 329 |
| 331 | ENPP3 | NP_005012.1 | Phosphatase (non-protein) | Y630 | EyVSGFGKAMR | SEQ ID NO: 330 |
| 332 | PPAP2A | NP_003702.2 | Phosphatase (non-protein) | Y168 | LSFySGHSSFSMYCMLFVALYLQA | SEQ ID NO: 331 |
| | | | | | RMK | |
| 333 | PPAP2A | NP_003702.2 | Phosphatase (non-protein) | Y185 | LSFYSGHSSFSMYCMLFVALyLQA | SEQ ID NO: 332 |
| | | | | | RMK | |
| 334 | INPP1 | NP_002185.1 | Phosphatase, lipid | Y225 | WGLSyMGTNMHSLQLTISRRNGSE | SEQ ID NO: 333 |
| | | | | | THTGNTGSEAAF | |
| 335 | INPP5D | NP_001017915.1 | Phosphatase, lipid | Y339 | SKDGSEDKFySHKKILQLIK | SEQ ID NO: 334 |
| 336 | INPP5D | NP_001017915.1 | Phosphatase, lipid | Y1161 | GRDyRDNTELPHHGK | SEQ ID NO: 335 |
| 337 | INPPL1 | NP_001558.2 | Phosphatase, lipid | Y831 | SMDGyESYGECVVALK | SEQ ID NO: 336 |
| 338 | INPPL1 | | Phosphatase, lipid | Y1135 | KTLSEVDyAPAGPAR | SEQ ID NO: 337 |
| 339 | INPPL1 | | Phosphatase, lipid | Y1162 | PRGLPSDyGRPLSFP | SEQ ID NO: 338 |
| 340 | PPM1G | NP_002698.1 | PHOSPHATASE; Phosphatase | Y364 | ALDMSyDHKPEDEVELARIK | SEQ ID NO: 339 |
| 341 | PPP1R12A | NP_002471.1 | PHOSPHATASE; Protein | Y549 | NSSVNEGSTyHK | SEQ ID NO: 340 |
| | | | phosphatase, regulatory | | | |
| | | | subunit; Protein phosphatase, | | | |
| | | | dual-specificity | | | |
| 342 | PPP1R12A | NP_002471.1 | PHOSPHATASE; Protein | Y762 | YSRTyDETYQR | SEQ ID NO: 341 |
| | | | phosphatase, regulatory | | | |
| | | | subunit; Protein phosphatase, | | | |
| | | | dual-specificity | | | |
| 343 | PPP1CB | NP_002700.1 | PHOSPHATASE; Protein | Y306 | YQyGGLNSGRPVTPPR | SEQ ID NO: 342 |
| | | | phosphatase, Ser/Thr | | | |
| | | | (non-receptor) | | | |
| 344 | PTPN18 | NP_055184.2 | PHOSPHATASE; Protein | Y314 | ENCAPLyDDALFLR | SEQ ID NO: 343 |
| | | | phosphatase, tyrosine | | | |
| | | | (non-receptor) | | | |
| 345 | PTPN7 | NP_002823.2 | PHOSPHATASE; Protein | Y149 | AQSQEDGDyINANYIR | SEQ ID NO: 344 |
| | | | phosphatase, tyrosine | | | |
| | | | (non-receptor) | | | |
| 346 | PTPN7 | NP_002823.2 | PHOSPHATASE; Protein | Y154 | AQSQEDGDYINANyIR | SEQ ID NO: 345 |
| | | | phosphatase, tyrosine | | | |
| | | | (non-receptor) | | | |
| 347 | PTPN6 | | PHOSPHATASE; Protein | Y543 | SEYGNITyPPAMKNA | SEQ ID NO: 346 |
| | | | phosphatase, tyrosine | | | |
| | | | (non-receptor) | | | |
| 348 | PTPRC | NP_002829.2 | PHOSPHATASE; Receptor | Y705 | VELSEINGDAGSNyINASYIDGFK | SEQ ID NO: 347 |
| | | | protein phosphatase, tyrosine | | EPR | |
| 349 | PRPRC | NP_002829.2 | PHOSPHATASE; Receptor | Y710 | VELSEINGDAGSNYINASyIDGFK | SEQ ID NO: 348 |
| | | | protein phosphatase, tyrosine | | EPR | |
| 350 | PTPRB | NP_002828.2 | PHOSPHATASE; Receptor | Y873 | VFPPFHLVNTATEyR | SEQ ID NO: 349 |
| | | | protein phosphatase, tyrosine | | | |
| 351 | PDE2A | NP_002590.1 | Phosphodiesterase | Y920 | GLPSNNSLDFLDEEyEVPDLDGTR | SEQ ID NO: 350 |
| | | | | | APINGCCSLDAE | |
| 352 | PDE8A | NP_002596.1 | Phosphodiesterase | Y194 | RYVENPNIMACyNELLQLEFGEVR | SEQ ID NO: 351 |
| | | | | | SQLKLR | |
| 353 | PLCG1 | NP_002651.2 | Phospholipase | Y481 | KLAEGSAYEEVPTSMMySENDISN | SEQ ID NO: 352 |
| | | | | | SIK | |
| 354 | PLCG2 | NP_002652.1 | Phospholipase | Y100 | AVRQKEDCCFTILyGTQFVLSTLS | SEQ ID NO: 353 |
| | | | | | LAADSK | |
| 355 | PLCG2 | NP_002652.1 | Phospholipase | Y680 | KREGSDSyAITFR | SEQ ID NO: 354 |
| 356 | PLD1 | NP_002653.1 | Phospholipase | Y757 | SAADWSAGIKyHEESIHAAYVHVI | SEQ ID NO: 355 |
| | | | | | ENSR | |
| 357 | PLD1 | NP_002653.1 | Phospholipase | Y766 | SAADWSAGIKYHEESIHAAyVHVI | SEQ ID NO: 356 |
| | | | | | ENSR | |
| 358 | PLD2 | | Phospholipase | Y580 | TPIYPyLLPK | SEQ ID NO: 357 |
| 359 | PLCG2 | | Phospholipase | Y1264 | VSNSKFyS | SEQ ID NO: 358 |
| 360 | CTSK | | Protease (non-proteasomal) | Y307 | ENWGNKGyILMARNK | SEQ ID NO: 359 |
| 361 | FAP | NP_004451.2 | Protease (non-proteasomal) | Y374 | DGyKHIHYIKDTVENAIQITSGK | SEQ ID NO: 360 |
| 362 | FAP | NP_004451.2 | Protease (non-proteasomal) | Y379 | DGYKHIHyIKDTVENAIQITSGK | SEQ ID NO: 361 |
| 363 | MMP9 | NP_004985.2 | Protease (non-proteasomal) | Y54 | QLAEEYLYRYGyTRVAEMR | SEQ ID NO: 362 |
| 364 | PSMA5 | NP_002781.2 | Protease (proteasomal | Y8 | SEyDRGVNTFSPEGR | SEQ ID NO: 363 |
| | | | subunit) | | | |
| 365 | PSMB4 | NP_002787.2 | Protease (proteasomal | Y107 | VNNSTMLGASGDYADFQyLK | SEQ ID NO: 364 |
| | | | subunit) | | | |
| 366 | LEPR | NP_001003679.1 | Receptor, cytokine | Y795 | yYIHDHFIPIEK | SEQ ID NO: 365 |
| 367 | LEPR | NP_001003679.1 | Receptor, cytokine | Y796 | YyIHDHFIPIEK | SEQ ID NO: 366 |
| 368 | MPL | | Receptor, cytokine | Y591 | SSQAQMDyRRPQPSC | SEQ ID NO: 367 |
| 369 | ADORA2B | NP_000667.1 | Receptor, GPCR | Y113 | yKSLVTGTRARGVIAVLWVLAFGI | SEQ ID NO: 368 |
| | | | | | GLTPFLGWNSK | |
| 370 | BAI3 | NP_001695.1 | Receptor, GPCR | Y1419 | ySDLDFEKVMHTRK | SEQ ID NO: 369 |
| 371 | SSTR4 | NP_001043.1 | Receptor, GPCR | Y347 | CCLLEGAGGAEEEPLDYyATALK | SEQ ID NO: 370 |
| 372 | TSHR | NP_000360.2 | Receptor, GPCR | Y643 | MAVLIFTDFICMAPISFyALSAIL | SEQ ID NO: 371 |
| | | | | | NKPLITVSNSK | |
| 373 | PROCR | NP_006395.2 | Receptor, misc. | Y171 | PERALWQADTQVTSGVVTFTLQQL | SEQ ID NO: 372 |
| | | | | | NAyNRTR | |
| 374 | CD19 | NP_001761.3 | Receptor, misc. | Y508 | GILyAAPQLR | SEQ ID NO: 373 |
| 375 | CD3G | NP_000064.1 | Receptor, misc. | Y160 | ASDKQTLLPNDQLyQPLKDREDDQ | SEQ ID NO: 374 |
| | | | | | YSHLQGNQLR | |
| 376 | CD3G | NP_000064.1 | Receptor, misc. | Y171 | ASDKQTLLPNDQLYQPLKDREDDQ | SEQ ID NO: 375 |
| | | | | | ySHLQGNQLR | |
| 377 | CD79B | NP_000617.1 | Receptor, misc. | Y196 | AGMEEDHTyEGLDIDQTATYEDIV | SEQ ID NO: 376 |
| | | | | | TLR | |
| 378 | CD79B | NP_000617.1 | Receptor, misc. | Y207 | AGMEEDHTYEGLDIDQTATyEDIV | SEQ ID NO: 377 |
| | | | | | TLR | |
| 379 | CR2 | NP_001006659.1 | Receptor, misc. | Y108 | YSSCPEPIVPGGYKIRGSTPyR | SEQ ID NO: 378 |
| 380 | GCER1G | | Receptor, misc. | Y65 | YEKSDGVyTGLSTRN | SEQ ID NO: 379 |
| 381 | KIR3DL2 | NP_006728.1 | Receptor, misc. | Y428 | TPLTDTSVyTELPNAEPR | SEQ ID NO: 380 |
| 382 | P2RY2 | NP_002555.2 | Receptor, misc. | Y118 | FLFYTNLyCSILFLTCISVHR | SEQ ID NO: 381 |
| 383 | ARNT | NP_001659.1 | Receptor, nuclear | Y561 | FSEIyHNINADQSK | SEQ ID NO: 382 |
| 384 | PPARA | NP_001001928.1 | Receptor, nuclear | Y136 | LKLVyDKCDRSCKIQKKNR | SEQ ID NO: 383 |
| 385 | XPO7 | NP_055839.2 | Receptor, protein | Y883 | LLLSIPHSDLLDyPK | SEQ ID NO: 384 |
| | | | translocating | | | |
| 386 | RANBP5 | NP_002262.3 | Receptor, protein | Y838 | RQDEDyDEQVEESLQDEDDNDVYI | SEQ ID NO: 385 |
| | | | translocating | | LTK | |
| 387 | CUTL1 | NP_001904.2 | Transcription factor | Y594 | KyLSLSPWDKATLSMGRLVLSNKM | SEQ ID NO: 386 |
| | | | | | AR | |
| 388 | CEBPZ | NP_005751.2 | Transcription factor | Y544 | yYTALYRKMLDPGLMTCSKQAMFL | SEQ ID NO: 387 |
| | | | | | NLVYKSLK | |
| 389 | CEBPZ | NP_005751.2 | Transcription factor | Y545 | YyTALYRKMLDPGLMTCSKQAMFL | SEQ ID NO: 388 |
| | | | | | NLVYKSLK | |
| 390 | CEBPZ | NP_005751.2 | Transcription factor | Y549 | YYTALyRKMLDPGLMTCSKQAMFL | SEQ ID NO: 389 |
| | | | | | NLVYSKSLK | |
| 391 | CEBPZ | NP_005751.2 | Transcription factor | Y571 | YYTALYRKMLDPGLMTCSKQAMFL | SEQ ID NO: 390 |
| | | | | | NLVyKSLK | |
| 392 | TCF3 | NP_003191.1 | Transcription factor | Y149 | GTSQyYPSYSGSSR | SEQ ID NO: 391 |
| 393 | TEV4 | NP_001977.1 | Transcription factor | Y416 | YYYEKGIMQKVAGERyVYK | SEQ ID NO: 392 |
| 394 | FUBP1 | NP_003893.2 | Transcription factor | Y60 | IGGDAGTSLNSNDYGyGGQK | SEQ ID NO: 393 |
| 395 | FUBP3 | NP_003925.1 | Transcription factor | Y81 | IDSIPHLNNSTPLVDPSVYGyGVQ | SEQ ID NO: 394 |
| | | | | | K | |
| 396 | FLI1 | NP_002008.2 | Transcription factor | Y222 | LSVKEDPSyDSVRR | SEQ ID NO: 395 |
| 397 | C21orf66 | NP_037461.2 | Transcription factor | Y609 | yYTSYKDAYIGCLPK | SEQ ID NO: 396 |
| 398 | C21orf66 | NP_037461.2 | Transcription factor | Y610 | YyTSYKDAYIGLCLPK | SEQ ID NO: 397 |
| 399 | C21orf66 | NP_037461.2 | Transcription factor | Y613 | YYTSyKDAYIGLCLPK | SEQ ID NO: 398 |
| 400 | GTF2H2 | NP_001506.1 | Transcription factor | Y289 | PSFSMAHLDGNTEPGLTLGGyFCP | SEQ ID NO: 399 |
| | | | | | QCRAK | |
| 401 | HKR2 | NP_862829.1 | Transcription factor | Y260 | FDLVDAYGTEPPyTYSGKR | SEQ ID NO: 400 |
| 402 | HKR2 | NP_862829.1 | Transcription factor | Y262 | FDLVDAYGREPPYTySGKR | SEQ ID NO: 401 |
| 403 | IRF5 | NP_002191.1 | Transcription factor | Y136 | IYEVCSNGPAPTDSQPPEDySFGA | SEQ ID NO: 402 |
| | | | | | GEEEEEEEELQR | |
| 404 | LMO4 | NP_006760.1 | Transcription factor | Y37 | CAGCGGKIADRFLLyAMDSYWHSR | SEQ ID NO: 403 |
| | | | | | CLK | |
| 405 | LMO4 | NP_006760.1 | Transcription factor | Y42 | CAGCGGKIADRFLLYAMDSyWHSR | SEQ ID NO: 404 |
| | | | | | CLK | |
| 406 | MXD1 | NP_002348.1 | Transcription factor | Y18 | MNIQMLLEAADyLERR | SEQ ID NO: 405 |
| 407 | ILF3 | NP_036350.2 | Transcription factor | Y867 | QGGYSQSNYNSPGSGQNySGPPSS | SEQ ID NO: 406 |
| | | | | | YQSSQGGYGR | |
| 408 | NFYA | NP_002496.1 | Transcription factor | Y266 | IPLPGAEMLEEEPLyVNAK | SEQ ID NO: 407 |
| 409 | RAI1 | NP_109590.3 | Transcription factor | Y305 | HHAQETLHyQNLAK | SEQ ID NO: 408 |
| 410 | SPEN | NP_055816.2 | Transcription factor | Y378 | FGKVTSVQIHGTSEERyGLVFFR | SEQ ID NO: 409 |
| 411 | STAT5A | NP_003143.2 | Transcription factor | Y22 | QMQVLyGQHFPEIVR | SEQ ID NO: 410 |
| 412 | STAT5A | NP_003143.2 | Transcription factor | Y90 | LGHyATQLQK | SEQ ID NO: 411 |
| 413 | STAT5A | NP_003143.2 | Transcription factor | Y114 | HILyNEQR | SEQ ID NO: 412 |
| 414 | SMAD2 | NP_001003652.1 | Transcription factor | Y102 | GLSTPNTIDQWDTTGLySFSEQTR | SEQ ID NO: 413 |
| | | | | | SL | |
| 415 | ETS1 | NP_005229.1 | Transcription factor | Y283 | VPSyDSFDSEDYPAALPNHKPK | SEQ ID NO: 414 |
| 416 | NSEP1 | NP_003550.2 | Transcription factor | Y145 | YAADRNHyRR | SEQ ID NO: 415 |
| 417 | NSEP1 | NP_004550.2 | Transcription factor | Y158 | NyQQNYQNSESGEKNEGSESAPEG | SEQ ID NO: 416 |
| | | | | | QAQQR | |
| 418 | NSEP1 | NP_004550.2 | Transcription factor | Y208 | RPQySNPPVQGEVMEGADNQGAGE | SEQ ID NO: 417 |
| | | | | | QGRPVR | |
| 419 | ZNF289 | | Transcription factor | Y445 | EVDAEyEAR | SEQ ID NO: 418 |
| 420 | GTF2I | NP_001509.2 | Transcription, | Y249 | SEDPDYyQYNIQGSHHSSEGNE | SEQ ID NO: 491 |
| | | | coactivator/corepressor | | | |
| 421 | GTF2I | NP_001509.2 | Transcription, | Y879 | APSyLEISSMR | SEQ ID NO: 420 |
| | | | coactivator/corepressor | | | |
| 422 | SKIIP | NP_036377.1 | Transcription, | Y176 | AADKLAPAQyIR | SEQ ID NO: 421 |
| | | | coactivator/corepressor | | | |
| 423 | SKIIP | NP_036377.1 | Transcription, | Y430 | DMDSGFAGGEDEIyNVYDQAWR | SEQ ID NO: 422 |
| | | | coactivator/corepressor | | | |
| 424 | SKIIP | NP_036377.1 | Transcription, | Y433 | GMDSGFAGGEDEIYNVyDQAWR | SEQ ID NO: 423 |
| | | | coactivator/corepressor | | | |
| 425 | TP53BP2 | NP_005417.1 | Transcription, | Y541 | NIySNSQGKPGSPEPE | SEQ ID NO: 424 |
| | | | coactivator/corepressor | | | |
| 426 | MPST | NP_001013454.1 | Transferase | Y272 | LCGKPDVPIyDGSWVEW | SEQ ID NO: 425 |
| 427 | AGXT | NP_000021.1 | Transferase | Y260 | MyHHTIPVISLYSLR | SEQ ID NO: 426 |
| 428 | ASNS | NP_001664.2 | Transferase | Y428 | VPFLDHRFSSyYLSLPPEMRIPK | SEQ ID NO: 427 |
| 429 | ASNS | NP_001664.2 | Transferase | Y429 | VPFLDHRFSSYyLSLPPEMRIPK | SEQ ID NO: 428 |
| 430 | BCAT1 | NP_005495.2 | Transferase | Y90 | LHyAVELGEGLK | SEQ ID NO: 429 |
| 431 | CHST1 | NP_003645.1 | Transferase | Y302 | YMLVRyEDLARNPMKK | SEQ ID NO: 430 |
| 432 | GSTP1 | NP_000843.1 | Transferase | Y8 | PPYTVVyFPVR | SEQ ID NO: 431 |
| 433 | GNPAT | NP_055051.1 | Transferase | Y60 | HVSDLKFAMKCYTPLVyKGITPCK | SEQ ID NO: 432 |
| 434 | HAS3 | NP_005320.2 | Transferase | Y520 | TAYCQDLFSETELAFLVSGAILyG | SEQ ID NO: 433 |
| | | | | | CY | |
| 435 | HAS3 | NP_005320.2 | Transferase | Y523 | TAYCQDLFSETELAFLVSGAILYG | SEQ ID NO: 434 |
| | | | | | Cy | |
| 436 | HADHB | NP_000174.1 | Transferase | Y244 | LEQDEyALR | SEQ ID NO: 435 |
| 437 | NMT1 | NP_066565.1 | Transferase | Y476 | LKFGIGDGNLQyYLYNKWK | SEQ ID NO: 436 |
| 438 | GLANT2 | NP_004472.1 | Transferase | Y177 | EIILVDDySNDPEDGALLGKIEKV | SEQ ID NO: 437 |
| | | | | | RVLRNDR | |
| 439 | POLR2H | NP_006223.2 | Transferase | Y75 | DGTLDDGEyNPTDDRPSRADQFE | SEQ ID NO: 438 |
| 440 | Shmt1 | | Transferase | Y28 | MLSQPLKDSDAEVySIIKK | SEQ ID NO: 439 |
| 441 | SHMT1 | NP_004160.3 | Transferase | Y34 | MLAQPLKDSDVEVYNIIK | SEQ ID NO: 440 |
| 442 | B4GALT5 | NP_004767.1 | Transferase | Y65 | DNVRTIGAQVyEQVLR | SEQ ID NO: 441 |
| 443 | B4GALT5 | NP_004767.1 | Transferase | Y262 | yMYLLPYTEFFGGVSGLTVEQFR | SEQ ID NO: 442 |
| 444 | B4GALT5 | NP_004767.1 | Transferase | Y264 | MYyLLPYTEFFGGVSGLTVEQFR | SEQ ID NO: 443 |
| 445 | B4GALT5 | NP_004767.1 | Transferase | Y278 | YMYLLPyTEFFGGVSGLTVEQFR | SEQ ID NO: 444 |
| 446 | EIF4EBP2 | NP_004087.1 | Translocation initiation | Y34 | TVAISDAAQLPHDyCTTPGGTLFS | SEQ ID NO: 445 |
| | | | complex | | TTPGGTR | |
| 447 | eIF2A | NP_114414.2 | Translocation initiation | Y250 | VIASTDVDKTGASyYGEQTLHY | SEQ ID NO: 446 |
| | | | complex | | | |
| 448 | IEF4G2 | NP_001409.1 | Translocation initiation | Y439 | SQGLSQLyHNQSQGLLSQLQGQSK | SEQ ID NO: 447 |
| | | | complex | | | |
| 449 | EIF3S8 | NP_003743.1 | Translocation initiation | Y881 | VFDHKQGTyGGYFR | SEQ ID NO: 448 |
| | | | complex | | | |
| 450 | EIF3S6 | NP_001559.1 | Translocation initiation | Y401 | LGHVVMGNNAVSPyQQVIEK | SEQ ID NO: 449 |
| | | | complex | | | |
| 451 | EEF1A1 | NP_001393.1 | Translocation initiation | Y86 | FETSKYyVTIIDAPGHR | SEQ ID NO: 450 |
| | | | complex | | | |
| 452 | EEF1D | NP_001951.2 | Translocation initiation | Y26 | FyEQMNGPVAGASR | SEQ ID NO: 451 |
| | | | complex | | | |
| 453 | EEF2 | NP_001952.1 | Translocation initiation | Y373 | CELLyEGPPDDEAAMGIK | SEQ ID NO: 452 |
| | | | complex | | | |
| 454 | EIF3S6IP | NP_057175.1 | Translocation initiation | Y23 | AAYDPYAYPSDyDMHTGDPKQDLA | SEQ ID NO: 453 |
| | | | complex | | YE | |
| 455 | EIF4B | NP_001408.2 | Translocation initiation | Y266 | YDDRGSRDyDRGYDSR | SEQ ID NO: 454 |
| | | | complex | | | |
| 456 | EIF4B | NP_001408.2 | Translocation initiation | Y270 | DRYDDRGSRDYDRGyDSR | SEQ ID NO: 455 |
| | | | complex | | | |
| 457 | EIF4B | NP_001408.2 | Translocation initiation | Y291 | DDDyRGGGDRYEDRYDRRDDR | SEQ ID NO: 456 |
| | | | complex | | | |
| 458 | EIF4B | NP_001408.2 | Translocation initiation | Y298 | DDDYRGGGDRyEDRYDRRDDR | SEQ ID NO: 457 |
| | | | complex | | | |
| 459 | EIF4B | NP_001408.2 | Translocation initiation | Y593 | SSASKyAALSVDGEDENEGEDYAE | SEQ ID NO: 458 |
| | | | complex | | | |
| 460 | IEF4B | NP_001408.2 | Translocation initiation | Y609 | SSASKYAALSVDGEDENEGEDyAE | SEQ ID NO: 459 |
| | | | complex | | | |
| 461 | EIF5 | NP_001960.2 | Translocation initiation | Y405 | VVySKAASVPKVE | SEQ ID NO: 460 |
| | | | complex | | | |
| 462 | RPS10 | NP_001005.1 | Translocation initiation | Y127 | LTRGEADRDTyRR | SEQ ID NO: 461 |
| | | | complex | | | |
| 463 | TAF15 | NP_003478.1 | Translation initiation | Y50 | SGYGQTTDSSYGQNySGY | SEQ ID NO: 462 |
| | | | complex; RNA binding protein | | | |
| 464 | TAF15 | NP_003478.1 | Translation initiation | Y132 | DQHQGSYDEQSNyDQQHDSY | SEQ ID NO: 463 |
| | | | complex; RNA binding protein | | | |
| 465 | TAF15 | NP_003478.1 | Translation initiation | Y139 | DQHQGSYDEQSNYDQQHDSySQNQ | SEQ ID NO: 464 |
| | | | complex; RNA binding protein | | QSY | |
| 466 | ABCD3 | NP_002849.1 | Transporter, ABC | Y267 | LRRPIGKMTITEQKYEGEYRyVNS | SEQ ID NO: 465 |
| | | | | | R | |
| 467 | TAP2 | NP_000535.3 | Transporter, ABC | Y693 | LAQLQEGQDLySR | SEQ ID NO: 466 |
| 468 | ATP1A2 | NP_000693.1 | Transporter, active | Y9 | EySPAATTAENGGGKKKQKEK | SEQ ID NO: 467 |
| 469 | SLC6A6 | NP_003034.2 | Transporter, active | Y598 | VKYLLTPREPNRWAVEREGATPyN | SEQ ID NO: 468 |
| | | | | | SR | |
| 470 | ATP6V0A2 | NP_036595.2 | Transporter, active | Y149 | NVEFEPTyEEFPSLESDSLLDYSC | SEQ ID NO: 469 |
| | | | | | MQR | |
| 471 | ATP6V0A2 | NP_036595.2 | Transporter, active | Y163 | NVEFEPTYEEFPSLESDSLLDySC | SEQ ID NO: 470 |
| | | | | | MQR | |
| 472 | SLC1A6 | NP_005062.1 | Transporter, facilitator | Y88 | yFSFPGELLMRMLQMLVLPLIVSS | SEQ ID NO: 471 |
| | | | | | LVTGMASLDNK | |
| 473 | SLC1A4 | NP_003029.2 | Transporter, facilitator | Y10 | SNETNGyLDSAQAGPAAGPGAPGT | SEQ ID NO: 472 |
| | | | | | AAGR | |
| 474 | SLC7A2 | NP_001008539.1 | Transporter, facilitator | Y621 | DENNEEDAyPDNVHAAAEEK | SEQ ID NO: 473 |
| 475 | APC | NP_000029.2 | Tumor suppressor | Y1078 | NQSTTYPVyTESTDDK | SEQ ID NO: 474 |
| 476 | BIRC6 | | Ubiquitin conjugating system | Y4260 | STEEQQLyWAKGTGF | SEQ ID NO: 475 |
| 477 | NYCBP2 | NP_055872.2 | Ubiquitin conjugating system | Y1238 | FSADTDILLGGLGLFGGRGEyTAK | SEQ ID NO: 476 |
| | | | | | IK | |
| 478 | PIAS1 | NP_057250.1 | Ubiquitin conjugating system | Y144 | LQKLPFyDLLDELIK | SEQ ID NO: 477 |
| 479 | CAND2 | XP_371617.2 | Ubiquitin conjugating system | Y965 | PSLVRDLLDDILPLLyQETK | SEQ ID NO: 478 |
| 480 | USP19 | NP_006668.1 | Ubiquitin conjugating system | Y853 | LTyARLAQLLEGYARYSVSVFQPP | SEQ ID NO: 479 |
| | | | | | FQPGR | |
| 481 | CUL5 | NP_003469.2 | Ubiquitin conjugating system | Y373 | FLTARDKAyKAVVNDATIFKLELP | SEQ ID NO: 480 |
| | | | | | LKQK | |

The short name for each protein in which a phosphorylation site has presently been identified is provided in Column A, and its SwissProt accession number (human) is provided Column B. The protein type/group into which each protein falls is provided in Column C. The identified tyrosine or serine residue at which phosphorylation occurs in a given protein is identified in Column D, and the amino acid sequence of the phosphorylation site encompassing the tyrosine residue is provided in Column E (lower case y=the tyrosine (identified in Column D)) at which phosphorylation occurs. Table 1 above is identical to FIG. 2, except that the latter includes the disease and cell type(s) in which the particular phosphorylation site was identified (Columns F and G).

The identification of these 480 phosphorylation sites is described in more detail in Part A below and in Example 1.

### Definitions

As used herein, the following terms have the meanings indicated:

“Antibody” or “antibodies” refers to all types of immunoglobulins, including IgG, IgM, IgA, IgD, and IgE, including F_{ab} or antigen-recognition fragments thereof, including chimeric, polyclonal, and monoclonal antibodies. The term “does not bind” with respect to an antibody's binding to one phospho-form of a sequence means does not substantially react with as compared to the antibody's binding to the other phospho-form of the sequence for which the antibody is specific.

“Leukemia-related signaling protein” means any protein (or poly-peptide derived therefrom) enumerated in Column A of Table 1/FIG. 2, which is disclosed herein as being phosphorylated in one or more leukemia cell line(s). Leukemia-related signaling proteins may be tyrosine kinases, such as Flt-3 or BCR-Abl, or serine/threonine kinases, or direct substrates of such kinases, or may be indirect substrates downstream of such kinases in signaling pathways. A Leukemia-related signaling protein may also be phosphorylated in other cell lines (non-leukemic) harboring activated kinase activity.

“Heavy-isotope labeled peptide” (used interchangeably with AQUA peptide) means a peptide comprising at least one heavy-isotope label, which is suitable for absolute quantification or detection of a protein as described in WO/03016861, “Absolute Quantification of Proteins and Modified Forms Thereof by Multistage Mass Spectrometry” (Gygi et al.), further discussed below.

“Protein” is used interchangeably with polypeptide, and includes protein fragments and domains as well as whole protein.

“Phosphorylatable amino acid” means any amino acid that is capable of being modified by addition of a phosphate group, and includes both forms of such amino acid.

“Phosphorylatable peptide sequence” means a peptide sequence comprising a phosphorylatable amino acid.

“Phosphorylation site-specific antibody” means an antibody that specifically binds a phosphorylatable peptide sequence/epitope only when phosphorylated, or only when not phosphorylated, respectively. The term is used interchangeably with “phospho-specific” antibody.

### A. Identification of Novel Leukemia-Related Protein Phosphorylation Sites.

The nearly 480 novel Leukemia-related signaling protein phosphorylation sites disclosed herein and listed in Table 1/FIG. 2 were discovered by employing the modified peptide isolation and characterization techniques described in “Immunoaffinity Isolation of Modified Peptides From Complex Mixtures,” U.S. Patent Publication No. 20030044848, Rush et al. (the teaching of which is hereby incorporated herein by reference, in its entirety) using cellular extracts from the following human Leukemia (AML, ALL, CML and CLL) derived cell lines and patient samples: Jurkat, K562, SEM, HT-93, CTV-1, MOLT15, CLL-9, H1993, OCL-ly3, KBM-3, UT-7, SUPT-13, MKPL-1, HU-3, M-07e, HU-3, EHEB, SU-DHL1, OCI-Iy1, DU-528, CMK, OCI-Iy8, ELF-153, OCI-Iy18, MEC-1, Karpas 299, CLL23LB4, OCI-Iy12, M01043, CLL-10, HL60, Molm 14, MV4-11, CLL-1202, EOL-1, CLL-19, CV-1, PL21; or from the following cell lines expressing activated BCR-Abl wild type and mutant kinases such as: Baf3-p210 BCR-Abl, Baf3-M351T-BCR-ABL, Baf3-E255K-BCR-Abl, Baf3-Y253F-BCR-Abl, Baf3-T315l-BCR-ABI, 3T3-v-Abl; or activated Flt3 kinase such as Baf3-FLT3 or FLT3-ITD; or JAK2 such as Baf3/Jak2; or mutant JAK2 V617F such as Baf3-V617F -JAK2, or Tyk2 such as Baf3/Tyk2; or TEL-FGFR3 such as Baf3-Tel/FGFR3; or TpoR such as Baf3/TpoR and Baf3/cc-TpoR-IV; or FGFR1 such as 293T-FGFR. The isolation and identification of phosphopeptides from these cell lines, using an immobilized general phosphotyrosine-specific antibody, or an antibody recognizing the phosphorylated motif PXpSP is described in detail in Example 1 below. In addition to the 80 previously unknown protein phosphorylation sites (tyrosine) discovered, many known phosphorylation sites were also identified (not described herein). The immunoaffinity/mass spectrometric technique described in the '848 Patent Publication (the “IAP” method)—and employed as described in detail in the Examples—is briefly summarized below.

The IAP method employed generally comprises the following steps: (a) a proteinaceous preparation (e.g. a digested cell extract) comprising phosphopeptides from two or more different proteins is obtained from an organism; (b) the preparation is contacted with at least one immobilized general phosphotyrosine-specific antibody; (c) at least one phosphopeptide specifically bound by the immobilized antibody in step (b) is isolated; and (d) the modified peptide isolated in step (c) is characterized by mass spectrometry (MS) and/or tandem mass spectrometry (MS-MS). Subsequently, (e) a search program (e.g. Sequest) may be utilized to substantially match the spectra obtained for the isolated, modified peptide during the characterization of step (d) with the spectra for a known peptide sequence. A quantification step employing, e.g. SILAC or AQUA, may also be employed to quantify isolated peptides in order to compare peptide levels in a sample to a baseline.

In the IAP method as employed herein, a general phosphotyrosine-specific monoclonal antibody (commercially available from Cell Signaling Technology, Inc., Beverly, Mass., Cat #9411 (p-Tyr-100)), was used in the immunoaffinity step to isolate the widest possible number of phospho-tyrosine containing peptides from the cell extracts.

Extracts from the following human Leukemia cell lines (ALL, AML, CLL, CML, respectively) were employed: Jurkat, K562, SEM, HT-93, CTV-1, MOLT15, CLL-9, H1993, OCL-Iy3, KBM-3, UT-7, SUPT-13, MKPL-1, HU-3, M-07e, HU-3, EHEB, SU-DHL1, OCI-Iy1, DU-528, CMK, OCI-Iy8, ELF-153, OCI-Iy18, MEC-1, Karpas 299, CLL23LB4, OCI-Iy12, M01043, CLL-10, HL60, Molm 14, MV4-11, CLL-1202, EOL-1, CLL-19, CV-1, PL21; or from the following cell lines expressing activated BCR-Abl wild type and mutant kinases such as: Baf3-p210 BCR-Abl, Baf3-M351T-BCR-ABL, Baf3-E255K-BCR-Abl, Baf3-Y253F-BCR-Abl, Baf3-T3151-BCR-ABI, 3T3-v-Abl; or activated Flt3 kinase such as Baf3-FLT3 or FLT3-ITD; or JAK2 such as Baf3/Jak2; or mutant JAK2 V617F such as Baf3-V617F -JAK2, orTyk2 such as Baf3/Tyk2; or TEL-FGFR3 such as Baf3-Tel/FGFR3; or TpoR such as Baf3/TpoR and Baf3/cc-TpoR-IV; or FGFR1 such as 293T-FGFR.

As described in more detail in the Examples, lysates were prepared from these cells line and digested with trypsin after treatment with DTT and iodoacetamide to alkylate cysteine residues. Before the immunoaffinity step, peptides were pre-fractionated by reversed-phase solid phase extraction using Sep-Pak C₁₈ columns to separate peptides from other cellular components. The solid phase extraction cartridges were eluted with varying steps of acetonitrile. Each lyophilized peptide fraction was redissolved in PBS and treated with a phosphotyrosine antibody (P-Tyr-100, CST#9411) immobilized on protein G-Sepharose or Protein A-Sepharose. Immunoaffinity-purified peptides were eluted with 0.1% TFA and a portion of this fraction was concentrated with Stage or Zip tips and analyzed by LC-MS/MS, using a ThermoFinnigan LTQ ion trap mass spectrometer. Peptides were eluted from a 10 cm×75 μm reversed-phase column with a 45-min linear gradient of acetonitrile. MS/MS spectra were evaluated using the program Sequest with the NCBI human protein database.

This revealed a total of nearly 480 novel tyrosine phosphorylation sites in signaling pathways affected by kinase activation or active in leukemia cells. The identified phosphorylation sites and their parent proteins are enumerated in Table 1/FIG. 2. The tyrosine (human sequence) at which phosphorylation occurs is provided in Column D, and the peptide sequence encompassing the phosphorylatable tyrosine residue at the site is provided in Column E. FIG. 2 also shows the particular type of leukemic disease (see Column G) and cell line(s) (see Column F) in which a particular phosphorylation site was discovered.

As a result of the discovery of these phosphorylation sites, phospho-specific antibodies and AQUA peptides for the detection of and quantification of these sites and their parent proteins may now be produced by standard methods, described below. These new reagents will prove highly useful in, e.g., studying the signaling pathways and events underlying the progression of leukemias and the identification of new biomarkers and targets for diagnosis and treatment of such diseases.

### B. Antibodies and Cell Lines

Isolated phosphorylation site-specific antibodies that specifically bind a Leukemia-related signaling protein disclosed in Column A of Table 1 only when phosphorylated (or only when not phosphorylated) at the corresponding amino acid and phosphorylation site listed in Columns D and E of Table 1/FIG. 2 may now be produced by standard antibody production methods, such as anti-peptide antibody methods, using the phosphorylation site sequence information provided in Column E of Table 1. For example, two previously unknown BCR kinase phosphorylation sites (tyrosines 58 and 231) (see Rows 225-226 of Table 1/FIG. 2) are presently disclosed. Thus, antibodies that specifically bind either of these novel BCR kinase sites can now be produced, e.g. by immunizing an animal with a peptide antigen comprising all or part of the amino acid sequence encompassing the respective phosphorylated residue (e.g. a peptide antigen comprising the sequence set forth in Row 225, Column E, of Table 1 (SEQ ID NO: 224) (which encompasses the phosphorylated tyrosine at position 58 in BCR), to produce an antibody that only binds BCR kinase when phosphorylated at that site.

Polyclonal antibodies of the invention may be produced according to standard techniques by immunizing a suitable animal (e.g., rabbit, goat, etc.) with a peptide antigen corresponding to the Leukemia-related phosphorylation site of interest (i.e. a phosphorylation site enumerated in Column E of Table 1, which comprises the corresponding phosphorylatable amino acid listed in Column D of Table 1), collecting immune serum from the animal, and separating the polyclonal antibodies from the immune serum, in accordance with known procedures. For example, a peptide antigen corresponding to all or part of the novel ATM kinase phosphorylation site disclosed herein (SEQ ID NO: 229=EVEGTSYHESLyNALQSLRDREFYESLKYAR, encompassing phosphorylated tyrosine 2129 (see Row 230 of Table 1)) may be used to produce antibodies that only bind ATM when phosphorylated at Tyr2129. Similarly, a peptide comprising all or part of any one of the phosphorylation site sequences provided in Column E of Table 1 may employed as an antigen to produce an antibody that only binds the corresponding protein listed in Column A of Table 1 when phosphorylated (or when not phosphorylated) at the corresponding residue listed in Column D. If an antibody that only binds the protein when phosphorylated at the disclosed site is desired, the peptide antigen includes the phosphorylated form of the amino acid. Conversely, if an antibody that only binds the protein when not phosphorylated at the disclosed site is desired, the peptide antigen includes the non-phosphorylated form of the amino acid.

Peptide antigens suitable for producing antibodies of the invention may be designed, constructed and employed in accordance with well-known techniques. See, e.g., ANTIBODIES: A LABORATORY MANUAL, Chapter 5, p. 75-76, Harlow & Lane Eds., Cold Spring Harbor Laboratory (1988); Czernik, Methods In Enzymology, 201: 264-283 (1991); Merrifield, J. Am. Chem. Soc. 85: 21-49 (1962)).

It will be appreciated by those of skill in the art that longer or shorter phosphopeptide antigens may be employed. See Id. For example, a peptide antigen may comprise the full sequence disclosed in Column E of Table 1/FIG. 2, or it may comprise additional amino acids flanking such disclosed sequence, or may comprise of only a portion of the disclosed sequence immediately flanking the phosphorylatable amino acid (indicated in Column E by an uppercase “Y”). Typically, a desirable peptide antigen will comprise four or more amino acids flanking each side of the phosphorylatable amino acid and encompassing it. Polyclonal antibodies produced as described herein may be screened as further described below.

Monoclonal antibodies of the invention may be produced in a hybridoma cell line according to the well-known technique of Kohler and Milstein. See Nature 265: 495-97 (1975); Kohler and Milstein, Eur. J Immunol. 6: 511 (1976); see also, CURRENT PROTOCOLS IN MOLECULAR BIOLOGY, Ausubel et al. Eds. (1989). Monoclonal antibodies so produced are highly specific, and improve the selectivity and specificity of diagnostic assay methods provided by the invention. For example, a solution containing the appropriate antigen may be injected into a mouse or other species and, after a sufficient time (in keeping with conventional techniques), the animal is sacrificed and spleen cells obtained. The spleen cells are then immortalized by fusing them with myeloma cells, typically in the presence of polyethylene glycol, to produce hybridoma cells. Rabbit fusion hybridomas, for example, may be produced as described in U.S. Pat. No. 5,675,063, C. Knight, Issued Oct. 7, 1997. The hybridoma cells are then grown in a suitable selection media, such as hypoxanthine-aminopterin-thymidine (HAT), and the supernatant screened for monoclonal antibodies having the desired specificity, as described below. The secreted antibody may be recovered from tissue culture supernatant by conventional methods such as precipitation, ion exchange or affinity chromatography, or the like.

Monoclonal Fab fragments may also be produced in Escherichia coli by recombinant techniques known to those skilled in the art. See, e.g., W. Huse, Science 246:1275-81 (1989); Mullinax et al., Proc. Nat'l Acad. Sci. 87: 8095 (1990). If monoclonal antibodies of one isotype are preferred for a particular application, particular isotypes can be prepared directly, by selecting from the initial fusion, or prepared secondarily, from a parental hybridoma secreting a monoclonal antibody of different isotype by using the sib selection technique to isolate class-switch variants (Steplewski, et al., Proc. Nat'l. Acad. Sci., 82: 8653 (1985); Spira et al., J. Immunol. Methods, 74: 307 (1984)).

The preferred epitope of a phosphorylation-site specific antibody of the invention is a peptide fragment consisting essentially of about 8 to 17 amino acids including the phosphorylatable tyrosine, wherein about 3 to 8 amino acids are positioned on each side of the phosphorylatable tyrosine (for example, the MELK tyrosine 438 phosphorylation site sequence disclosed in Row 244, Column E of Table 1), and antibodies of the invention thus specifically bind a target Leukemia-related signaling polypeptide comprising such epitopic sequence. Particularly preferred epitopes bound by the antibodies of the invention comprise all or part of a phosphorylatable site sequence listed in Column E of Table 1, including the phosphorylatable amino acid.

Included in the scope of the invention are equivalent non-antibody molecules, such as protein binding domains or nucleic acid aptamers, which bind, in a phospho-specific manner, to essentially the same phosphorylatable epitope to which the phospho-specific antibodies of the invention bind. See, e.g., Neuberger et al., Nature 312: 604 (1984). Such equivalent non-antibody reagents may be suitably employed in the methods of the invention further described below.

Antibodies provided by the invention may be any type of immunoglobulins, including IgG, IgM, IgA, IgD, and IgE, including F_{ab} or antigen-recognition fragments thereof. The antibodies may be monoclonal or polyclonal and may be of any species of origin, including (for example) mouse, rat, rabbit, horse, or human, or may be chimeric antibodies. See, e.g., M. Walker et al., Molec. ImmunoL 26: 403-11 (1989); Morrision et al., Proc. Nat'l. Acad. Sci. 81: 6851 (1984); Neuberger et al., Nature 312: 604 (1984)). The antibodies may be recombinant monoclonal antibodies produced according to the methods disclosed in U.S. Pat. No. 4,474,893 (Reading) or U.S. Pat. No. 4,816,567 (Cabilly et al.) The antibodies may also be chemically constructed by specific antibodies made according to the method disclosed in U.S. Pat. No. 4,676,980 (Segel et al.)

The invention also provides immortalized cell lines that produce an antibody of the invention. For example, hybridoma clones, constructed as described above, that produce monoclonal antibodies to the Leukemia-related signaling protein phosphorylation sties disclosed herein are also provided. Similarly, the invention includes recombinant cells producing an antibody of the invention, which cells may be constructed by well known techniques; for example the antigen combining site of the monoclonal antibody can be cloned by PCR and single-chain antibodies produced as phage-displayed recombinant antibodies or soluble antibodies in E. coli (see, e.g., ANTIBODY ENGINEERING PROTOCOLS, 1995, Humana Press, Sudhir Paul editor.)

Phosphorylation site-specific antibodies of the invention, whether polyclonal or monoclonal, may be screened for epitope and phospho-specificity according to standard techniques. See, e.g. Czemik et al., Methods in Enzymology, 201: 264-283 (1991). For example, the antibodies may be screened against the phospho and non-phospho peptide library by ELISA to ensure specificity for both the desired antigen (i.e. that epitope including a phosphorylation site sequence enumerated in Column E of Table 1) and for reactivity only with the phosphorylated (or non-phosphorylated) form of the antigen. Peptide competition assays may be carried out to confirm lack of reactivity with other phospho-epitopes on the given Leukemia-related signaling protein. The antibodies may also be tested by Western blotting against cell preparations containing the signaling protein, e.g. cell lines over-expressing the target protein, to confirm reactivity with the desired phosphorylated epitope/target.

Specificity against the desired phosphorylated epitope may also be examined by constructing mutants lacking phosphorylatable residues at positions outside the desired epitope that are known to be phosphorylated, or by mutating the desired phospho-epitope and confirming lack of reactivity. Phosphorylation-site specific antibodies of the invention may exhibit some limited cross-reactivity to related epitopes in non-target proteins. This is not unexpected as most antibodies exhibit some degree of cross-reactivity, and anti-peptide antibodies will often cross-react with epitopes having high homology to the immunizing peptide. See, e.g., Czernik, supra. Cross-reactivity with non-target proteins is readily characterized by Western blotting alongside markers of known molecular weight. Amino acid sequences of cross-reacting proteins may be examined to identify sites highly homologous to the Leukemia-related signaling protein epitope for which the antibody of the invention is specific.

In certain cases, polyclonal antisera may exhibit some undesirable general cross-reactivity to phosphotyrosine itself, which may be removed by further purification of antisera, e.g. over a phosphotyramine column. Antibodies of the invention specifically bind their target protein (i.e. a protein listed in Column A of Table 1) only when phosphorylated (or only when not phosphorylated, as the case may be) at the site disclosed in corresponding Columns D/E, and do not (substantially) bind to the other form (as compared to the form for which the antibody is specific).

Antibodies may be further characterized via immunohistochemical (IHC) staining using normal and diseased tissues to examine Leukemia-related phosphorylation and activation status in diseased tissue. IHC may be carried out according to well-known techniques. See, e.g., ANTIBODIES: A LABORATORY MANUAL, Chapter 10, Harlow & Lane Eds., Cold Spring Harbor Laboratory (1988). Briefly, paraffin-embedded tissue (e.g. tumor tissue) is prepared for immunohistochemical staining by deparaffinizing tissue sections with xylene followed by ethanol; hydrating in water then PBS; unmasking antigen by heating slide in sodium citrate buffer; incubating sections in hydrogen peroxide; blocking in blocking solution; incubating slide in primary antibody and secondary antibody; and finally detecting using ABC avidin/biotin method according to manufacturer's instructions.

Antibodies may be further characterized by flow cytometry carried out according to standard methods. See Chow et al., Cytometry (Communications in Clinical Cytometry) 46: 72-78 (2001). Briefly and by way of example, the following protocol for cytometric analysis may be employed: samples may be centrifuged on Ficoll gradients to remove erythrocytes, and cells may then be fixed with 2% paraformaldehyde for 10 minutes at 37° C. followed by permeabilization in 90% methanol for 30 minutes on ice. Cells may then be stained with the primary phosphorylation-site specific antibody of the invention (which detects a Leukemia-related signal transduction protein enumerated in Table 1), washed and labeled with a fluorescent-labeled secondary antibody. Additional fluorochrome-conjugated marker antibodies (e.g. CD45, CD34) may also be added at this time to aid in the subsequent identification of specific hematopoietic cell types. The cells would then be analyzed on a flow cytometer (e.g. a Beckman Coulter FC500) according to the specific protocols of the instrument used.

Antibodies of the invention may also be advantageously conjugated to fluorescent dyes (e.g. Alexa488, PE) for use in multi-parametric analyses along with other signal transduction (phospho-CrkL, phospho-Erk 1/2) and/or cell marker (CD34) antibodies.

Phosphorylation-site specific antibodies of the invention specifically bind to a human Leukemia-related signal transduction protein or polypeptide only when phosphorylated at a disclosed site, but are not limited only to binding the human species, per se. The invention includes antibodies that also bind conserved and highly homologous or identical phosphorylation sites in respective Leukemia-related proteins from other species (e.g. mouse, rat, monkey, yeast), in addition to binding the human phosphorylation site. Highly homologous or identical sites conserved in other species can readily be identified by standard sequence comparisons, such as using BLAST, with the human Leukemia-related signal transduction protein phosphorylation sites disclosed herein.

### C. Heavy-Isotope Labeled Peptides (AQUA Peptides).

The novel Leukemia-related signaling protein phosphorylation sites disclosed herein now enable the production of corresponding heavy-isotope labeled peptides for the absolute quantification of such signaling proteins (both phosphorylated and not phosphorylated at a disclosed site) in biological samples. The production and use of AQUA peptides for the absolute quantification of proteins (AQUA) in complex mixtures has been described. See WO/03016861, “Absolute Quantification of Proteins and Modified Forms Thereof by Multistage Mass Spectrometry,” Gygi et al. and also Gerber et al. Proc. Nati. Acad. Sci. U.S.A. 100: 6940-5 (2003) (the teachings of which are hereby incorporated herein by reference, in their entirety).

The AQUA methodology employs the introduction of a known quantity of at least one heavy-isotope labeled peptide standard (which has a unique signature detectable by LC-SRM chromatography) into a digested biological sample in order to determine, by comparison to the peptide standard, the absolute quantity of a peptide with the same sequence and protein modification in the biological sample. Briefly, the AQUA methodology has two stages: peptide internal standard selection and validation and method development; and implementation using validated peptide internal standards to detect and quantify a target protein in sample. The method is a powerful technique for detecting and quantifying a given peptide/protein within a complex biological mixture, such as a cell lysate, and may be employed, e.g., to quantify change in protein phosphorylation as a result of drug treatment, or to quantify differences in the level of a protein in different biological states.

Generally, to develop a suitable internal standard, a particular peptide (or modified peptide) within a target protein sequence is chosen based on its amino acid sequence and the particular protease to be used to digest. The peptide is then generated by solid-phase peptide synthesis such that one residue is replaced with that same residue containing stable isotopes (¹³C, ¹⁵N). The result is a peptide that is chemically identical to its native counterpart formed by proteolysis, but is easily distinguishable by MS via a 7-Da mass shift. A newly synthesized AQUA internal standard peptide is then evaluated by LC-MS/MS. This process provides qualitative information about peptide retention by reverse-phase chromatography, ionization efficiency, and fragmentation via collision-induced dissociation. Informative and abundant fragment ions for sets of native and internal standard peptides are chosen and then specifically monitored in rapid succession as a function of chromatographic retention to form a selected reaction monitoring (LC-SRM) method based on the unique profile of the peptide standard.

The second stage of the AQUA strategy is its implementation to measure the amount of a protein or modified protein from complex mixtures. Whole cell lysates are typically fractionated by SDS-PAGE gel electrophoresis, and regions of the gel consistent with protein migration are excised. This process is followed by in-gel proteolysis in the presence of the AQUA peptides and LC-SRM analysis. (See Gerber et al. supra.) AQUA peptides are spiked in to the complex peptide mixture obtained by digestion of the whole cell lysate with a proteolytic enzyme and subjected to immunoaffinity purification as described above. The retention time and fragmentation pattern of the native peptide formed by digestion (e.g. trypsinization) is identical to that of the AQUA internal standard peptide determined previously; thus, LC-MS/MS analysis using an SRM experiment results in the highly specific and sensitive measurement of both internal standard and analyte directly from extremely complex peptide mixtures. Because an absolute amount of the AQUA peptide is added (e.g. 250 fmol), the ratio of the areas under the curve can be used to determine the precise expression levels of a protein or phosphorylated form of a protein in the original cell lysate. In addition, the internal standard is present during in-gel digestion as native peptides are formed, such that peptide extraction efficiency from gel pieces, absolute losses during sample handling (including vacuum centrifugation), and variability during introduction into the LC-MS system do not affect the determined ratio of native and AQUA peptide abundances.

An AQUA peptide standard is developed for a known phosphorylation site sequence previously identified by the IAP-LC-MS/MS method within a target protein. One AQUA peptide incorporating the phosphorylated form of the particular residue within the site may be developed, and a second AQUA peptide incorporating the non-phosphorylated form of the residue developed. In this way, the two standards may be used to detect and quantify both the phosphorylated and non-phosphorylated forms of the site in a biological sample.

Peptide internal standards may also be generated by examining the primary amino acid sequence of a protein and determining the boundaries of peptides produced by protease cleavage. Alternatively, a protein may actually be digested with a protease and a particular peptide fragment produced can then sequenced. Suitable proteases include, but are not limited to, serine proteases (e.g. trypsin, hepsin), metallo proteases (e.g. PUMP1), chymotrypsin, cathepsin, pepsin, thermolysin, carboxypeptidases, etc.

A peptide sequence within a target protein is selected according to one or more criteria to optimize the use of the peptide as an internal standard. Preferably, the size of the peptide is selected to minimize the chances that the peptide sequence will be repeated elsewhere in other non-target proteins. Thus, a peptide is preferably at least about 6 amino acids. The size of the peptide is also optimized to maximize ionization frequency. Thus, peptides longer than about 20 amino acids are not preferred. The preferred ranged is about 7 to 15 amino acids. A peptide sequence is also selected that is not likely to be chemically reactive during mass spectrometry, thus sequences comprising cysteine, tryptophan, or methionine are avoided.

A peptide sequence that does not include a modified region of the target region may be selected so that the peptide internal standard can be used to determine the quantity of all forms of the protein. Alternatively, a peptide internal standard encompassing a modified amino acid may be desirable to detect and quantify only the modified form of the target protein. Peptide standards for both modified and unmodified regions can be used together, to determine the extent of a modification in a particular sample (i.e. to determine what fraction of the total amount of protein is represented by the modified form). For example, peptide standards for both the phosphorylated and unphosphorylated form of a protein known to be phosphorylated at a particular site can be used to quantify the amount of phosphorylated form in a sample.

The peptide is labeled using one or more labeled amino acids (i.e. the label is an actual part of the peptide) or less preferably, labels may be attached after synthesis according to standard methods. Preferably, the label is a mass-altering label selected based on the following considerations: The mass should be unique to shift fragment masses produced by MS analysis to regions of the spectrum with low background; the ion mass signature component is the portion of the labeling moiety that preferably exhibits a unique ion mass signature in MS analysis; the sum of the masses of the constituent atoms of the label is preferably uniquely different than the fragments of all the possible amino acids. As a result, the labeled amino acids and peptides are readily distinguished from unlabeled ones by the ion/mass pattern in the resulting mass spectrum. Preferably, the ion mass signature component imparts a mass to a protein fragment that does not match the residue mass for any of the 20 natural amino acids.

The label should be robust under the fragmentation conditions of MS and not undergo unfavorable fragmentation. Labeling chemistry should be efficient under a range of conditions, particularly denaturing conditions, and the labeled tag preferably remains soluble in the MS buffer system of choice. The label preferably does not suppress the ionization efficiency of the protein and is not chemically reactive. The label may contain a mixture of two or more isotopically distinct species to generate a unique mass spectrometric pattern at each labeled fragment position. Stable isotopes, such as ²H, ¹³C, ¹⁵N, ¹⁷O, ¹⁸O, or ³⁴S, are among preferred labels. Pairs of peptide internal standards that incorporate a different isotope label may also be prepared. Preferred amino acid residues into which a heavy isotope label may be incorporated include leucine, proline, valine, and phenylalanine.

Peptide internal standards are characterized according to their mass-to-charge (m/z) ratio, and preferably, also according to their retention time on a chromatographic column (e.g. an HPLC column). Internal standards that co-elute with unlabeled peptides of identical sequence are selected as optimal internal standards. The internal standard is then analyzed by fragmenting the peptide by any suitable means, for example by collision-induced dissociation (CID) using, e.g., argon or helium as a collision gas. The fragments are then analyzed, for example by multi-stage mass spectrometry (MSⁿ) to obtain a fragment ion spectrum, to obtain a peptide fragmentation signature. Preferably, peptide fragments have significant differences in m/z ratios to enable peaks corresponding to each fragment to be well separated, and a signature that is unique for the target peptide is obtained. If a suitable fragment signature is not obtained at the first stage, additional stages of MS are performed until a unique signature is obtained.

Fragment ions in the MS/MS and MS³ spectra are typically highly specific for the peptide of interest, and, in conjunction with LC methods, allow a highly selective means of detecting and quantifying a target peptide/protein in a complex protein mixture, such as a cell lysate, containing many thousands or tens of thousands of proteins. Any biological sample potentially containing a target protein/peptide of interest may be assayed. Crude or partially purified cell extracts are preferably employed. Generally, the sample has at least 0.01 mg of protein, typically a concentration of 0.1-10 mg/mL, and may be adjusted to a desired buffer concentration and pH.

A known amount of a labeled peptide internal standard, preferably about 10 femtomoles, corresponding to a target protein to be detected/quantified is then added to a biological sample, such as a cell lysate. The spiked sample is then digested with one or more protease(s) for a suitable time period to allow digestion. A separation is then performed (e.g. by HPLC, reverse-phase HPLC, capillary electrophoresis, ion exchange chromatography, etc.) to isolate the labeled internal standard and its corresponding target peptide from other peptides in the sample. Microcapillary LC is a preferred method.

Each isolated peptide is then examined by monitoring of a selected reaction in the MS. This involves using the prior knowledge gained by the characterization of the peptide internal standard and then requiring the MS to continuously monitor a specific ion in the MS/MS or MSⁿ spectrum for both the peptide of interest and the internal standard. After elution, the area under the curve (AUC) for both peptide standard and target peptide peaks are calculated. The ratio of the two areas provides the absolute quantification that can be normalized for the number of cells used in the analysis and the protein's molecular weight, to provide the precise number of copies of the protein per cell. Further details of the AQUA methodology are described in Gygi et al., and Gerber et al. supra.

In accordance with the present invention, AQUA internal peptide standards (heavy-isotope labeled peptides) may now be produced, as described above, for any of the nearly 480 novel Leukemia-related signaling protein phosphorylation sites disclosed herein (see Table 1/FIG. 2). Peptide standards for a given phosphorylation site (e.g. the tyrosine 187 in BLK—see Row 271 of Table 1) may be produced for both the phosphorylated and non-phosphorylated forms of the site (e.g. see BLK site sequence in Column E, Row 271 of Table 1 (SEQ ID NO: 270) and such standards employed in the AQUA methodology to detect and quantify both forms of such phosphorylation site in a biological sample.

AQUA peptides of the invention may comprise all, or part of, a phosphorylation site peptide sequence disclosed herein (see Column E of Table 1/FIG. 2). In a preferred embodiment, an AQUA peptide of the invention comprises a phosphorylation site sequence disclosed herein in Table 1/FIG. 2. For example, an AQUA peptide of the invention for detection/quantification of SYK kinase when phosphorylated at tyrosine Y296 may comprise the sequence IKSySFPKPGHR (y=phosphotyrosine), which comprises phosphorylatable tyrosine 296 (see Row 274, Column E; (SEQ ID NO: 273)). Heavy-isotope labeled equivalents of the peptides enumerated in Table 1/FIG. 2 (both in phosphorylated and unphosphorylated form) can be readily synthesized and their unique MS and LC-SRM signature determined, so that the peptides are validated as AQUA peptides and ready for use in quantification experiments.

The phosphorylation site peptide sequences disclosed herein (see Column E of Table 1/FIG. 2) are particularly well suited for development of corresponding AQUA peptides, since the IAP method by which they were identified (see Part A above and Example 1) inherently confirmed that such peptides are in fact produced by enzymatic digestion (trypsinization) and are in fact suitably fractionated/ionized in MS/MS. Thus, heavy-isotope labeled equivalents of these peptides (both in phosphorylated and unphosphorylated form) can be readily synthesized and their unique MS and LC-SRM signature determined, so that the peptides are validated as AQUA peptides and ready for use in quantification experiments.

Accordingly, the invention provides heavy-isotope labeled peptides (AQUA peptides) for the detection and/or quantification of any of the Leukemia-related phosphorylation sites disclosed in Table 1/FIG. 2 (see Column E) and/or their corresponding parent proteins/polypeptides (see Column A). A phosphopeptide sequence comprising any of the phosphorylation sequences listed in Table 1 may be considered a preferred AQUA peptide of the invention. For example, an AQUA peptide comprising the sequence VMNATAyGISK (SEQ ID NO:285) (where y may be either phosphotyrosine or tyrosine, and where V=labeled valine (e.g. ¹⁴C)) is provided for the quantification of phosphorylated (or non-phosphorylated) FLT3 kinase (Tyr630) in a biological sample (see Row 286 of Table 1, tyrosine 630 being the phosphorylatable residue within the site). However, it will be appreciated that a larger AQUA peptide comprising a disclosed phosphorylation site sequence (and additional residues downstream or upstream of it) may also be constructed. Similarly, a smaller AQUA peptide comprising less than all of the residues of a disclosed phosphorylation site sequence (but still comprising the phosphorylatable residue enumerated in Column D of Table 1/FIG. 2) may alternatively be constructed. Such larger or shorter AQUA peptides are within the scope of the present invention, and the selection and production of preferred AQUA peptides may be carried out as described above (see Gygi et al., Gerber et al. supra.).

Certain particularly preferred subsets of AQUA peptides provided by the invention are described above (corresponding to particular protein types/groups in Table 1, for example, Tyrosine Protein Kinases or Protein Phosphatases). Example 4 is provided to further illustrate the construction and use, by standard methods described above, of exemplary AQUA peptides provided by the invention. For example, the above-described AQUA peptides corresponding to the both the phosphorylated and non-phosphorylated forms of the disclosed FLT3 kinase tyrosine 630 phosphorylation site (see Row 286 of Table 1/FIG. 2) may be used to quantify the amount of phosphorylated FLT3 (Tyr630) in a biological sample, e.g. a tumor cell sample (or a sample before or after treatment with a test drug).

AQUA peptides of the invention may also be employed within a kit that comprises one or multiple AQUA peptide(s) provided herein (for the quantification of a Leukemia-related signal transduction protein disclosed in Table 1/FIG. 2), and, optionally, a second detecting reagent conjugated to a detectable group. For example, a kit may include AQUA peptides for both the phosphorylated and non-phosphorylated form of a phosphorylation site disclosed herein. The reagents may also include ancillary agents such as buffering agents and protein stabilizing agents, e.g., polysaccharides and the like. The kit may further include, where necessary, other members of the signal-producing system of which system the detectable group is a member (e.g., enzyme substrates), agents for reducing background interference in a test, control reagents, apparatus for conducting a test, and the like. The test kit may be packaged in any suitable manner, typically with all elements in a single container along with a sheet of printed instructions for carrying out the test.

AQUA peptides provided by the invention will be highly useful in the further study of signal transduction anomalies underlying cancer, including leukemias, and in identifying diagnostic/bio-markers of these diseases, new potential drug targets, and/or in monitoring the effects of test compounds on Leukemia-related signal transduction proteins and pathways.

### D. Immunoassay Formats

Antibodies provided by the invention may be advantageously employed in a variety of standard immunological assays (the use of AQUA peptides provided by the invention is described separately above). Assays may be homogeneous assays or heterogeneous assays. In a homogeneous assay the immunological reaction usually involves a phosphorylation-site specific antibody of the invention), a labeled analyte, and the sample of interest. The signal arising from the label is modified, directly or indirectly, upon the binding of the antibody to the labeled analyte. Both the immunological reaction and detection of the extent thereof are carried out in a homogeneous solution. Immunochemical labels that may be employed include free radicals, radioisotopes, fluorescent dyes, enzymes, bacteriophages, coenzymes, and so forth.

In a heterogeneous assay approach, the reagents are usually the specimen, a phosphorylation-site specific antibody of the invention, and suitable means for producing a detectable signal. Similar specimens as described above may be used. The antibody is generally immobilized on a support, such as a bead, plate or slide, and contacted with the specimen suspected of containing the antigen in a liquid phase. The support is then separated from the liquid phase and either the support phase or the liquid phase is examined for a detectable signal employing means for producing such signal. The signal is related to the presence of the analyte in the specimen. Means for producing a detectable signal include the use of radioactive labels, fluorescent labels, enzyme labels, and so forth. For example, if the antigen to be detected contains a second binding site, an antibody which binds to that site can be conjugated to a detectable group and added to the liquid phase reaction solution before the separation step. The presence of the detectable group on the solid support indicates the presence of the antigen in the test sample. Examples of suitable immunoassays are the radioimmunoassay, immunofluorescence methods, enzyme-linked immunoassays, and the like.

Immunoassay formats and variations thereof that may be useful for carrying out the methods disclosed herein are well known in the art. See generally E. Maggio, Enzyme-Immunoassay, (1980) (CRC Press, Inc., Boca Raton, Fla.); see also, e.g., U.S. Pat. No. 4,727,022 (Skold et al., “Methods for Modulating Ligand-Receptor Interactions and their Application”); U.S. Pat. No. 4,659,678 (Forrest et al., “Immunoassay of Antigens”); U.S. Pat. No. 4,376,110 (David et al., “Immunometric Assays Using Monoclonal Antibodies”). Conditions suitable for the formation of reagent-antibody complexes are well described. See id. Monoclonal antibodies of the invention may be used in a “two-site” or “sandwich” assay, with a single cell line serving as a source for both the labeled monoclonal antibody and the bound monoclonal antibody. Such assays are described in U.S. Pat. No. 4,376,110. The concentration of detectable reagent should be sufficient such that the binding of a target Leukemia-related signal transduction protein is detectable compared to background.

Phosphorylation site-specific antibodies disclosed herein may be conjugated to a solid support suitable for a diagnostic assay (e.g., beads, plates, slides or wells formed from materials such as latex or polystyrene) in accordance with known techniques, such as precipitation. Antibodies, or other target protein or target site-binding reagents, may likewise be conjugated to detectable groups such as radiolabels (e.g., ³⁵S, ¹²⁵I, ¹³¹I), enzyme labels (e.g., horseradish peroxidase, alkaline phosphatase), and fluorescent labels (e.g., fluorescein) in accordance with known techniques.

Antibodies of the invention may also be optimized for use in a flow cytometry (FC) assay to determine the activation/phosphorylation status of a target Leukemia-related signal transduction protein in patients before, during, and after treatment with a drug targeted at inhibiting phosphorylation at such a protein at the phosphorylation site disclosed herein. For example, bone marrow cells or peripheral blood cells from patients may be analyzed by flow cytometry for target Leukemia-related signal transduction protein phosphorylation, as well as for markers identifying various hematopoietic cell types. In this manner, activation status of the malignant cells may be specifically characterized. Flow cytometry may be carried out according to standard methods. See, e.g. Chow et al., Cytometry (Communications in Clinical Cytometry) 46: 72-78 (2001). Briefly and by way of example, the following protocol for cytometric analysis may be employed: fixation of the cells with 1% para-formaldehyde for 10 minutes at 37° C. followed by permeabilization in 90% methanol for 30 minutes on ice. Cells may then be stained with the primary antibody (a phospho-specific antibody of the invention), washed and labeled with a fluorescent-labeled secondary antibody. Alternatively, the cells may be stained with a fluorescent-labeled primary antibody. The cells would then be analyzed on a flow cytometer (e.g. a Beckman Coulter EPICS-XL) according to the specific protocols of the instrument used. Such an analysis would identify the presence of activated Leukemia-related signal transduction protein(s) in the malignant cells and reveal the drug response on the targeted protein.

Alternatively, antibodies of the invention may be employed in immunohistochemical (IHC) staining to detect differences in signal transduction or protein activity using normal and diseased tissues. IHC may be carried out according to well-known techniques. See, e.g., ANTIBODIES: A LABORATORY MANUAL, supra. Briefly, paraffin-embedded tissue (e.g. tumor tissue) is prepared for immunohistochemical staining by deparaffinizing tissue sections with xylene followed by ethanol; hydrating in water then PBS; unmasking antigen by heating slide in sodium citrate buffer; incubating sections in hydrogen peroxide; blocking in blocking solution; incubating slide in primary antibody and secondary antibody; and finally detecting using ABC avidin/biotin method according to manufacturer's instructions.

Antibodies of the invention may be also be optimized for use in other clinically-suitable applications, for example bead-based multiplex-type assays, such as IGEN, Luminex™ and/or Bioplex™ assay formats, or otherwise optimized for antibody arrays formats, such as reversed-phase array applications (see, e.g. Paweletz et al., Oncogene 20(16): 1981-89 (2001)). Accordingly, in another embodiment, the invention provides a method for the multiplex detection of Leukemia-related protein phosphorylation in a biological sample, the method comprising utilizing two or more antibodies or AQUA peptides of the invention to detect the presence of two or more phosphorylated Leukemia-related signaling proteins enumerated in Column A of Table 1/FIG. 2. In one preferred embodiment, two to five antibodies or AQUA peptides of the invention are employed in the method. In another preferred embodiment, six to ten antibodies or AQUA peptides of the invention are employed, while in another preferred embodiment eleven to twenty such reagents are employed.

Antibodies and/or AQUA peptides of the invention may also be employed within a kit that comprises at least one phosphorylation site-specific antibody or AQUA peptide of the invention (which binds to or detects a Leukemia-related signal transduction protein disclosed in Table 1/FIG. 2), and, optionally, a second antibody conjugated to a detectable group. In some embodies, the kit is suitable for multiplex assays and comprises two or more antibodies or AQUA peptides of the invention, and in some embodiments, comprises two to five, six to ten, or eleven to twenty reagents of the invention. The kit may also include ancillary agents such as buffering agents and protein stabilizing agents, e.g., polysaccharides and the like. The kit may further include, where necessary, other members of the signal-producing system of which system the detectable group is a member (e.g., enzyme substrates), agents for reducing background interference in a test, control reagents, apparatus for conducting a test, and the like. The test kit may be packaged in any suitable manner, typically with all elements in a single container along with a sheet of printed instructions for carrying out the test.

The following Examples are provided only to further illustrate the invention, and are not intended to limit its scope, except as provided in the claims appended hereto. The present invention encompasses modifications and variations of the methods taught herein which would be obvious to one of ordinary skill in the art.

### EXAMPLE 1

### Isolation of Phosphotyrosine-Containing Peptides from Extracts of Leukemia Cell Lines and Identification of Novel Phosphorylation Sites

In order to discover previously unknown Leukemia-related signal transduction protein phosphorylation sites, IAP isolation techniques were employed to identify phosphotyrosine- and/or phosphoserine-containing peptides in cell extracts from the following human Leukemia cell lines and patient cell lines: Jurkat, K562, SEM, HT-93, CTV-1, MOLT15, CLL-9, H1993, OCL-Iy3, KBM-3, UT-7, SUPT-13, MKPL-1, HU-3, M-07e, HU-3, EHEB, SU-DHL1, OCI-Iy1, DU-528, CMK, OCI-Iy8, ELF-153, OCI-Iy18, MEC-1, Karpas 299, CLL23LB4, OCI-Iy12, M01043, CLL-10, HL60, Molm 14, MV4-11, CLL-1202, EOL-1, CLL-19, CV-1, PL21; or from the following cell lines expressing activated BCR-Abl wild type and mutant kinases such as: Baf3-p210 BCR-Abl, Baf3-M351T-BCR-ABL, Baf3-E255K-BCR-Abl, Baf3-Y253F-BCR-Abl, Baf3-T3151-BCR-ABI, 3T3-v-Abl; or activated Flt3 kinase such as Baf3-FLT3 or FLT3-ITD; or JAK2 such as Baf3/Jak2; or mutant JAK2 V617F such as Baf3-V617F -JAK2, or Tyk2 such as Baf3/Tyk2; or TEL-FGFR3 such as Baf3-Tel/FGFR3; or TpoR such as Baf3/TpoR and Baf3/cc-TpoR-IV; or FGFR1 such as 293T-FGFR.

Tryptic phosphotyrosine—containing peptides were purified and analyzed from extracts of each of the 29 cell lines mentioned above, as follows. Cells were cultured in DMEM medium or RPMI 1640 medium supplemented with 10% fetal bovine serum and penicillin/streptomycin.

Suspension cells were harvested by low speed centrifugation. After complete aspiration of medium, cells were resuspended in 1 mL lysis buffer per 1.25×10⁸ cells (20 mM HEPES pH 8.0, 9 M urea, 1 mM sodium vanadate, supplemented or not with 2.5 mM sodium pyro-phosphate, 1 mM β-glycerol-phosphate) and sonicated.

Sonicated cell lysates were cleared by centrifugation at 20,000×g, and proteins were reduced with DTT at a final concentration of 4.1 mM and alkylated with iodoacetamide at 8.3 mM. For digestion with trypsin, protein extracts were diluted in 20 mM HEPES pH 8.0 to a final concentration of 2 M urea and soluble TLCK-trypsin (Worthington) was added at 10-20 μg/mL. Digestion was performed for 1-2 days at room temperature.

Trifluoroacetic acid (TFA) was added to protein digests to a final concentration of 1 %, precipitate was removed by centrifugation, and digests were loaded onto Sep-Pak C₁₈ columns (Waters) equilibrated with 0.1% TFA. A column volume of 0.7-1.0 ml was used per 2×10⁸ cells. Columns were washed with 15 volumes of 0.1 % TFA, followed by 4 volumes of 5% acetonitrile (MeCN) in 0.1% TFA. Peptide fraction I was obtained by eluting columns with 2 volumes each of 8, 12, and 15% MeCN in 0.1% TFA and combining the eluates. Fractions II and III were a combination of eluates after eluting columns with 18, 22, 25% MeCN in 0.1 % TFA and with 30, 35, 40% MeCN in 0.1% TFA, respectively. All peptide fractions were lyophilized.

Peptides from each fraction corresponding to 2×10⁸ cells were dissolved in 1 ml of IAP buffer (20 mM Tris/HCl or 50 mM MOPS pH 7.2, 10 mM sodium phosphate, 50 mM NaCl) and insoluble matter (mainly in peptide fractions III) was removed by centrifugation. IAP was performed on each peptide fraction separately. The phosphotyrosine monoclonal antibody P-Tyr-100 (Cell Signaling Technology, Inc., catalog number 9411) was coupled at 4 mg/ml beads to protein G or protein A agarose (Roche), respectively. Immobilized antibody (15 μl, 60 μg) was added as 1:1 slurry in IAP buffer to 1 ml of each peptide fraction, and the mixture was incubated overnight at 4° C. with gentle rotation. The immobilized antibody beads were washed three times with 1 ml IAP buffer and twice with 1 ml water, all at 4° C. Peptides were eluted from beads by incubation with 75 μl of 0.1% TFA at room temperature for 10 minutes.

Alternatively, one single peptide fraction was obtained from Sep-Pak C18 columns by elution with 2 volumes each of 10%, 15%, 20%, 25%, 30%, 35% and 40% acetonitirile in 0.1% TFA and combination of all eluates. IAP on this peptide fraction was performed as follows: After lyophilization, peptide was dissolved in 1.4 ml IAP buffer (MOPS pH 7.2, 10 mM sodium phosphate, 50 mM NaCl) and insoluble matter was removed by centrifugation. Immobilized antibody (40 μl, 160 μg) was added as 1:1 slurry in IAP buffer, and the mixture was incubated overnight at 4° C. with gentle shaking. The immobilized antibody beads were washed three times with 1 ml IAP buffer and twice with 1 ml water, all at 4° C. Peptides were eluted from beads by incubation with 55 μl of 0.15% TFA at room temperature for 10 min (eluate 1), followed by a wash of the beads (eluate 2) with 45 μl of 0.15% TFA. Both eluates were combined.

### Analysis bv LC-MS/MS Mass Spectrometry

40 μl or more of IAP eluate were purified by 0.2 μl StageTips or ZipTips. Peptides were eluted from the microcolumns with 1 μl of 40% MeCN, 0.1% TFA (fractions I and II) or 1 μl of 60% MeCN, 0.1% TFA (fraction III) into 7.6 μl of 0.4% acetic acid/0.005% heptafluorobutyric acid. This sample was loaded onto a 10 cm×75 μm PicoFrit capillary column (New Objective) packed with Magic C18 AQ reversed-phase resin (Michrom Bioresources) using a Famos autosampler with an inert sample injection valve (Dionex). The column was then developed with a 45-min linear gradient of acetonitrile delivered at 200 nl/min (Ultimate, Dionex), and tandem mass spectra were collected in a data-dependent manner with an LTQ ion trap mass spectrometer essentially as described by Gygi et al., supra.

### Database Analysis & Assignments.

MS/MS spectra were evaluated using TurboSequest in the Sequest Browser package (v. 27, rev. 12) supplied as part of BioWorks 3.0 (ThermoFinnigan). Individual MS/MS spectra were extracted from the raw data file using the Sequest Browser program CreateDta, with the following settings: bottom MW, 700; top MW, 4,500; minimum number of ions, 20; minimum TIC, 4×10⁵; and precursor charge state, unspecified. Spectra were extracted from the beginning of the raw data file before sample injection to the end of the eluting gradient. The lonQuest and VuDta programs were not used to further select MS/MS spectra for Sequest analysis. MS/MS spectra were evaluated with the following TurboSequest parameters: peptide mass tolerance, 2.5; fragment ion tolerance, 0.0; maximum number of differential amino acids per modification, 4; mass type parent, average; mass type fragment, average;

maximum number of internal cleavage sites, 10; neutral losses of water and ammonia from b and y ions were considered in the correlation analysis. Proteolytic enzyme was specified except for spectra collected from elastase digests.

Searches were performed against the NCBI human protein database (as released on Feb. 23, 2004 and containing 27, 175 protein sequences). Cysteine carboxamidomethylation was specified as a static modification, and phosphorylation was allowed as a variable modification on serine, threonine, and tyrosine residues or on tyrosine residues alone. It was determined that restricted phosphorylation to tyrosine residues had little effect on the number of phosphorylation sites assigned. Furthermore it should be noted that certain peptides were originally isolated in mouse and later normalized to human sequences as shown by Tablel/FIG. 2.

In proteomics research, it is desirable to validate protein identifications based solely on the observation of a single peptide in one experimental result, in order to indicate that the protein is, in fact, present in a sample. This has led to the development of statistical methods for validating peptide assignments, which are not yet universally accepted, and guidelines for the publication of protein and peptide identification results (see Carr et al., Mol. Cell Proteomics 3: 531-533 (2004)), which were followed in this Example. However, because the immunoaffinity strategy separates phosphorylated peptides from unphosphorylated peptides, observing just one phosphopeptide from a protein is a common result, since many phosphorylated proteins have only one tyrosine-phosphorylated site. For this reason, it is appropriate to use additional criteria to validate phosphopeptide assignments. Assignments are likely to be correct if any of these additional criteria are met: (i) the same sequence is assigned to co-eluting ions with different charge states, since the MS/MS spectrum changes markedly with charge state; (ii) the site is found in more than one peptide sequence context due to sequence overlaps from incomplete proteolysis or use of proteases other than trypsin; (iii) the site is found in more than one peptide sequence context due to homologous but not identical protein isoforms; (iv) the site is found in more than one peptide sequence context due to homologous but not identical proteins among species; and (v) sites validated by MS/MS analysis of synthetic phosphopeptides corresponding to assigned sequences, since the ion trap mass spectrometer produces highly reproducible MS/MS spectra. The last criterion is routinely employed to confirm novel site assignments of particular interest.

All spectra and all sequence assignments made by Sequest were imported into a relational database. The following Sequest scoring thresholds were used to select phosphopeptide assignments that are likely to be correct: RSp<6, XCorr≧2.2, and DeltaCN>0.099. Further, the assigned sequences could be accepted or rejected with respect to accuracy by using the following conservative, two-step process.

In the first step, a subset of high-scoring sequence assignments should be selected by filtering for XCorr values of at least 1.5 for a charge state of +1, 2.2 for +2, and 3.3 for +3, allowing a maximum RSp value of 10. Assignments in this subset should be rejected if any of the following criteria were satisfied: (i) the spectrum contains at least one major peak (at least 10% as intense as the most intense ion in the spectrum) that can not be mapped to the assigned sequence as an a, b, or y ion, as an ion arising from neutral-loss of water or ammonia from a b or y ion, or as a multiply protonated ion; (ii) the spectrum does not contain a series of b or y ions equivalent to at least six uninterrupted residues; or (iii) the sequence is not observed at least five times in all the studies conducted (except for overlapping sequences due to incomplete proteolysis or use of proteases other than trypsin).

In the second step, assignments with below-threshold scores should be accepted if the low-scoring spectrum shows a high degree of similarity to a high-scoring spectrum collected in another study, which simulates a true reference library-searching strategy.

### EXAMPLE 2

### Production of Phospho-Specific Polyclonal Antibodies for the Detection of Leukemia-related Signaling Protein Phosphorylation

Polyclonal antibodies that specifically bind a Leukemia-related signal transduction protein only when phosphorylated at the respective phosphorylation site disclosed herein (see Table 1/FIG. 2) are produced according to standard methods by first constructing a synthetic peptide antigen comprising the phosphorylation site sequence and then immunizing an animal to raise antibodies against the antigen, as further described below. Production of exemplary polyclonal antibodies is provided below.

A. MELK (tyrosine 438).

A 15 amino acid phospho-peptide antigen, SAVKNEEy*FMFPEPK (where y*=phosphotyrosine) that corresponds to the sequence encompassing the tyrosine 438 phosphorylation site in human MELK kinase (see Row 244 of Table 1; SEQ ID NO: 243), plus cysteine on the C-terminal for coupling, is constructed according to standard synthesis techniques using, e.g., a Rainin/Protein Technologies, Inc., Symphony peptide synthesizer. See ANTIBODIES: A LABORATORY MANUAL, supra.; Merrifield, supra. This peptide is then coupled to KLH and used to immunize animals to produce (and subsequently screen) phospho-specific MELK(tyr438 ) polyclonal antibodies as described in Immunization/ Screening below.

B. SIT1 (tyrosine 127).

A 15 amino acid phospho-peptide antigen, AEEVMCy*TSLQLRP (where y*=phosphotyrosine) that corresponds to the sequence encompassing the tyrosine 127 phosphorylation site in human SIT1 adaptor/scaffold protein (see Row 38 of Table 1 (SEQ ID NO: 37)), plus cysteine on the C-terminal for coupling, is constructed according to standard synthesis techniques using, e.g., a Rainin/Protein Technologies, Inc., Symphony peptide synthesizer. See ANTIBODIES: A LABORATORY MANUAL, supra.; Merrifield, supra. This peptide is then coupled to KLH and used to immunize animals to produce (and subsequently screen) phospho-specific SIT1(tyr127) polyclonal antibodies as described in Immunization/Screening below.

C. PECAM1 (tyrosine 663).

A 13 amino acid phospho-peptide antigen, MEANSHy*GHNDDV (where y*=phosphotyrosine) that corresponds to the sequence encompassing the tyrosine 663 phosphorylation site in human PECAM1 adhesion protein (see Row 73 of Table 1 (SEQ ID NO: 72), plus cysteine on the C-terminal for coupling, is constructed according to standard synthesis techniques using, e.g., a Rainin/Protein Technologies, Inc., Symphony peptide synthesizer. See ANTIBODIES: A LABORATORY MANUAL, supra.; Merrifield, supra. This peptide is then coupled to KLH and used to immunize animals to produce (and subsequently screen) phospho-specific PECAM1 (tyr663) antibodies as described in Immunization/Screening below.

### Immunization/Screening.

A synthetic phospho-peptide antigen as described in A-C above is coupled to KLH, and rabbits are injected intradermally (ID) on the back with antigen in complete Freunds adjuvant (500 μg antigen per rabbit). The rabbits are boosted with same antigen in incomplete Freund adjuvant (250 μg antigen per rabbit) every three weeks. After the fifth boost, bleeds are collected. The sera are purified by Protein A-affinity chromatography by standard methods (see ANTIBODIES: A LABORATORY MANUAL, Cold Spring Harbor, supra.). The eluted immunoglobulins are further loaded onto a non-phosphorylated synthetic peptide antigen-resin Knotes column to pull out antibodies that bind the non-phosphorylated form of the phosphorylation site. The flow through fraction is collected and applied onto a phospho-synthetic peptide antigen-resin column to isolate antibodies that bind the phosphorylated form of the site. After washing the column extensively, the bound antibodies (i.e. antibodies that bind a phosphorylated peptide described in A-C above, but do not bind the non-phosphorylated form of the peptide) are eluted and kept in antibody storage buffer.

The isolated antibody is then tested for phospho-specificity using Western blot assay using an appropriate cell line that expresses (or overexpresses) target phospho-protein (i.e. phosphorylated MELK, SIT1 or PECAM1), for example, SEM, Jurkat and MKPL-1 cells, respectively. Cells are cultured in DMEM or RPMI supplemented with 10% FCS. Cell are collected, washed with PBS and directly lysed in cell lysis buffer. The protein concentration of cell lysates is then measured. The loading buffer is added into cell lysate and the mixture is boiled at 100° C. for 5 minutes. 20 μl (10 μg protein) of sample is then added onto 7.5% SDS-PAGE gel.

A standard Western blot may be performed according to the Immunoblotting Protocol set out in the CELL SIGNALING TECHNOLOGY, INC. 2003-04 Catalogue, p. 390. The isolated phospho-specific antibody is used at dilution 1:1000. Phosphorylation-site specificity of the antibody will be shown by binding of only the phosphorylated form of the target protein. Isolated phospho-specific polyclonal antibody does not (substantially) recognize the target protein when not phosphorylated at the appropriate phosphorylation site in the non-stimulated cells (e.g. PECAM1 is not bound when not phosphorylated at tyrosine 663).

In order to confirm the specificity of the isolated antibody, different cell lysates containing various phosphorylated signal transduction proteins other than the target protein are prepared. The Western blot assay is performed again using these cell lysates. The phospho-specific polyclonal antibody isolated as described above is used (1:1000 dilution) to test reactivity with the different phosphorylated non-target proteins on Western blot membrane. The phospho-specific antibody does not significantly cross-react with other phosphorylated signal transduction proteins, although occasionally slight binding with a highly homologous phosphorylation-site on another protein may be observed. In such case the antibody may be further purified using affinity chromatography, or the specific immunoreactivity cloned by rabbit hybridoma technology.

### EXAMPLE 3

### Production of Phospho-Specific Monoclonal Antibodies for the Detection of Leukemia-Related Signaling Protein Phosphorylation

Monoclonal antibodies that specifically bind a Leukemia-related signal transduction protein only when phosphorylated at the respective phosphorylation site disclosed herein (see Table 1/FIG. 2) are produced according to standard methods by first constructing a synthetic peptide antigen comprising the phosphorylation site sequence and then immunizing an animal to raise antibodies against the antigen, and harvesting spleen cells from such animals to produce fusion hybridomas, as further described below. Production of exemplary monoclonal antibodies is provided below.

A. TSC2 (tyrosine 1736).

A 10 amino acid phospho-peptide antigen, PTDIy*PSKW (where y*=phosphotyrosine) that corresponds to the sequence encompassing the tyrosine 1736 phosphorylation site in human TSC2 GTPase activating protein (see Row 87 of Table 1 (SEQ ID NO: 86)), plus cysteine on the C-terminal for coupling, is constructed according to standard synthesis techniques using, e.g., a Rainin/Protein Technologies, Inc., Symphony peptide synthesizer. See ANTIBODIES: A LABORATORY MANUAL, supra.; Merrifield, supra. This peptide is then coupled to KLH and used to immunize animals and harvest spleen cells for generation (and subsequent screening) of phospho-specific monoclonal TSC2(tyr1736) antibodies as described in Immunization/Fusion/Screening below.

B. CD84 (tyrosine 279).

A 10 amino acid phospho-peptide antigen, ESRIy*DEIL (where y*=phosphotyrosine) that corresponds to the sequence encompassing the tyrosine 279 phosphorylation site in human CD84 cell surface protein (see Row 93 of Table 1 (SEQ ID NO: 92)), plus cysteine on the C-terminal for coupling, is constructed according to standard synthesis techniques using, e.g., a Rainin/Protein Technologies, Inc., Symphony peptide synthesizer. See ANTIBODIES: A LABORATORY MANUAL, supra.; Merrifield, supra. This peptide is then coupled to KLH and used to immunize animals and harvest spleen cells for generation (and subsequent screening) of phospho-specific monoclonal CD84(tyr279) antibodies as described in Immunization/Fusion/Screening below.

C. STAT5A (tyrosine 22).

A 14 amino acid phospho-peptide antigen, QMQVLy*GQHFPIEV (where Y*=phosphotyrosine) that corresponds to the sequence encompassing the tyrosine 22 phosphorylation site in human STAT5A transcription factor (see Row 411 of Table 1 (SEQ ID NO: 410)), plus cysteine on the C-terminal for coupling, is constructed according to standard synthesis techniques using, e.g., a Rainin/Protein Technologies, Inc., Symphony peptide synthesizer. See ANTIBODIES: A LABORATORY MANUAL, supra.; Merrifield, supra. This peptide is then coupled to KLH and used to immunize animals and harvest spleen cells for generation (and subsequent screening) of phospho-specific monoclonal STAT5A (tyr22) antibodies as described in Immunization/Fusion/Screening below.

### Immunization/Fusion/Screening.

A synthetic phospho-peptide antigen as described in A-C above is coupled to KLH, and BALB/C mice are injected intradermally (ID) on the back with antigen in complete Freunds adjuvant (e.g. 50 μg antigen per mouse). The mice are boosted with same antigen in incomplete Freund adjuvant (e.g. 25 μg antigen per mouse) every three weeks. After the fifth boost, the animals are sacrificed and spleens are harvested.

Harvested spleen cells are fused to SP2/0 mouse myeloma fusion partner cells according to the standard protocol of Kohler and Milstein (1975). Colonies originating from the fusion are screened by ELISA for reactivity to the phospho-peptide and non-phospho-peptide forms of the antigen and by Western blot analysis (as described in Example 1 above). Colonies found to be positive by ELISA to the phospho-peptide while negative to the non-phospho-peptide are further characterized by Western blot analysis. Colonies found to be positive by Western blot analysis are subcloned by limited dilution. Mouse ascites are produced from a single clone obtained from subcloning, and tested for phospho-specificity (against the TSC2, CD84, or STAT5A phospho-peptide antigen, as the case may be) on ELISA. Clones identified as positive on Western blot analysis using cell culture supernatant as having phospho-specificity, as indicated by a strong band in the induced lane and a weak band in the uninduced lane of the blot, are isolated and subcloned as clones producing monoclonal antibodies with the desired specificity.

Ascites fluid from isolated clones may be further tested by Western blot analysis. The ascites fluid should produce similar results on Western blot analysis as observed previously with the cell culture supernatant, indicating phospho-specificity against the phosphorylated target (e.g. STAT5A phosphorylated at tyrosine 22).

### EXAMPLE 4

### Production and Use of AQUA Peptides for the Quantification of Leukemia-Related Signaling Protein Phosphorylation

Heavy-isotope labeled peptides (AQUA peptides (internal standards)) for the detection and quantification of a Leukemia-related signal transduction protein only when phosphorylated at the respective phosphorylation site disclosed herein (see Table 1/FIG. 2) are produced according to the standard AQUA methodology (see Gygi et al., Gerber et al., supra.) methods by first constructing a synthetic peptide standard corresponding to the phosphorylation site sequence and incorporating a heavy-isotope label. Subsequently, the MSⁿ and LC-SRM signature of the peptide standard is validated, and the AQUA peptide is used to quantify native peptide in a biological sample, such as a digested cell extract. Production and use of exemplary AQUA peptides is provided below.

A. ZAP70 (tyrosine 164).

An AQUA peptide comprising the sequence, MPWy*HSSLTR (y*=phosphotyrosine; sequence incorporating ¹⁴C/¹⁵N-labeled leucine (indicated by bold L), which corresponds to the tyrosine 164 phosphorylation site in human ZAP70 kinase (see Row 284 in Table 1 (SEQ ID NO: 283)), is constructed according to standard synthesis techniques using, e.g., a Rainin/Protein Technologies, Inc., Symphony peptide synthesizer (see Merrifield, supra.) as further described below in Synthesis & MS/MS Signature. The ZAP70(tyr164) AQUA peptide is then spiked into a biological sample to quantify the amount of phosphorylated ZAP70(tyr164) in the sample, as further described below in Analysis & Quantification.

B. SCAP1 (tyrosine 142).

An AQUA peptide comprising the sequence GLFYy*YANEK (y*=phosphotyrosine; sequence incorporating ¹⁴C/¹⁵N-labeled leucine (indicated by bold L), which corresponds to the tyrosine 142 phosphorylation site in human SCAP1 adaptor/scaffold protein (see Row 42 in Table 1 (SEQ ID NO: 41)), is constructed according to standard synthesis techniques using, e.g., a Rainin/Protein Technologies, Inc., Symphony peptide synthesizer (see Merrifield, supra.) as further described below in Synthesis & MS/MS Signature. The SCAP1(tyr142) AQUA peptide is then spiked into a biological sample to quantify the amount of phosphorylated SCAP1(tyr142) in the sample, as further described below in Analysis & Quantification.

C. CFL1 (tyrosine 68)

An AQUA peptide comprising the sequence, GQTVDDPy*ATFVKML (y*=phosphotyrosine; sequence incorporating ¹⁴C/¹⁵N-labeled phenylalanine (indicated by bold F), which corresponds to the tyrosine 211 phosphorylation site in human CFL1 adaptor/scaffold protein (see Row 121 in Table 1 (SEQ ID NO: 120)), is constructed according to standard synthesis techniques using, e.g., a Rainin/Protein Technologies, Inc., Symphony peptide synthesizer (see Merrifield, supra.) as further described below in Synthesis & MS/MS Signature. The CFL1(tyr68) AQUA peptide is then spiked into a biological sample to quantify the amount of phosphorylated CFL1 (tyr68) in the sample, as further described below in Analysis & Quantification.

D. BLK (tyrosine 187).

An AQUA peptide comprising the sequence, CLDEGGy*YISPR (y*=phosphotyrosine; sequence incorporating ¹⁴C/¹⁵N-labeled proline (indicated by bold P), which corresponds to the tyrosine 187 phosphorylation site in human BLK kinase (see Row 271 in Table 1 (SEQ ID NO: 270)), is constructed according to standard synthesis techniques using, e.g., a Rainin/Protein Technologies, Inc., Symphony peptide synthesizer (see Merrifield, supra.) as further described below in Synthesis & MS/MS Signature. The BLK(tyr187) AQUA peptide is then spiked into a biological sample to quantify the amount of phosphorylated BLK(tyr187) in the sample, as further described below in Analysis & Quantification.

### Synthesis & MS/MS Spectra.

Fluorenylmethoxycarbonyl (Fmoc)-derivatized amino acid monomers may be obtained from AnaSpec (San Jose, Calif.). Fmoc-derivatized stable-isotope monomers containing one ¹⁵N and five to nine ¹³C atoms may be obtained from Cambridge Isotope Laboratories (Andover, Mass.). Preloaded Wang resins may be obtained from Applied Biosystems. Synthesis scales may vary from 5 to 25 μmol. Amino acids are activated in situ with 1-H-benzotriazolium, 1-bis(dimethylamino)methylene]-hexafluorophosphate (1-),3-oxide:1-hydroxybenzotriazole hydrate and coupled at a 5-fold molar excess over peptide. Each coupling cycle is followed by capping with acetic anhydride to avoid accumulation of one-residue deletion peptide by-products. After synthesis peptide-resins are treated with a standard scavenger-containing trifluoroacetic acid (TFA)-water cleavage solution, and the peptides are precipitated by addition to cold ether. Peptides (i.e. a desired AQUA peptide described in A-D above) are purified by reversed-phase C18 HPLC using standard TFA/acetonitrile gradients and characterized by matrix-assisted laser desorption ionization-time of flight (Biflex III, Bruker Daltonics, Billerica, Mass.) and ion-trap (ThermoFinnigan, LCQ DecaXP) MS.

MS/MS spectra for each AQUA peptide should exhibit a strong y-type ion peak as the most intense fragment ion that is suitable for use in an SRM monitoring/analysis. Reverse-phase microcapillary columns (0.1 Å˜150-220 mm) are prepared according to standard methods. An Agilent 1100 liquid chromatograph may be used to develop and deliver a solvent gradient [0.4% acetic acid/0.005% heptafluorobutyric acid (HFBA)/7% methanol and 0.4% acetic acid/0.005% HFBA/65% methanol/35% acetonitrile] to the microcapillary column by means of a flow splitter. Samples are then directly loaded onto the microcapillary column by using a FAMOS inert capillary autosampler (LC Packings, San Francisco) after the flow split. Peptides are reconstituted in 6% acetic acid/0.01% TFA before injection.

### Analysis & Quantification.

Target protein (e.g. a phosphorylated protein of A-D above) in a biological sample is quantified using a validated AQUA peptide (as described above). The IAP method is then applied to the complex mixture of peptides derived from proteolytic cleavage of crude cell extracts to which the AQUA peptides have been spiked in.

LC-SRM of the entire sample is then carried out. MS/MS may be performed by using a ThermoFinnigan (San Jose, Calif.) mass spectrometer (LTQ ion trap or TSQ Quantum triple quadrupole). On the LTQ, parent ions are isolated at 1.6 m/z width, the ion injection time being limited to 100 ms per microscan, with one microscans per peptide, and with an AGC setting of 1×10⁵; on the Quantum, Q1 is kept at 0.4 and Q3 at 0.8 m/z with a scan time of 200 ms per peptide. On both instruments, analyte and internal standard are analyzed in alternation within a previously known reverse-phase retention window; well-resolved pairs of internal standard and analyte are analyzed in separate retention segments to improve duty cycle. Data are processed by integrating the appropriate peaks in an extracted ion chromatogram (60.15 m/z from the fragment monitored) for the native and internal standard, followed by calculation of the ratio of peak areas multiplied by the absolute amount of internal standard (e.g., 500 fmol).

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. An isolated phosphorylation site-specific antibody that specifically binds a human Leukemia-related signaling protein selected from Column A of Table 1 only when phosphorylated at the tyrosine listed in corresponding Column D of Table 1, comprised within the phosphorylatable peptide sequence listed in corresponding Column E of Table 1 (SEQ ID NOs: 1-7, 9-14, 16, 19-21, 23, 26-30, 32-34, 36-45, 48-52, 56-58, 60-90, 93-119, 121-124, 129-151, 153-160, 163-180, 182-193, 195-197, 199-208, 210-221, 223-279, 281-294, 296-297, 299-316, 319-336, 339-345, 347-356, 358, 360-366, 368-378, 380-417, 419-438, 440-474, and 476-480), wherein said antibody does not bind said signaling protein when not phosphorylated at said tyrosine.

17. An isolated phosphorylation site-specific antibody that specifically binds a human Leukemia-related signaling protein selected from Column A of Table 1 only when not phosphorylated at the tyrosine listed in corresponding Column D of Table 1, comprised within the phosphorylatable peptide sequence listed in corresponding Column E of Table 1 (SEQ ID NOs: 1-7, 9-14, 16, 19-21, 23, 26-30, 32-34, 36-45, 48-52, 56-58, 60-90, 93-119, 121-124, 129-151, 153-160, 163-180, 182-193, 195-197, 199-208, 210-221, 223-279, 281-294, 296-297, 299-316, 319-336, 339-345, 347-356, 358, 360-366, 368-378, 380-417, 419-438, 440-474, and 476-480), wherein said antibody does not bind said signaling protein when phosphorylated at said tyrosine.

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. An isolated phosphorylation site-specific antibody according to claim 16, that specifically binds a human Leukemia-related signaling protein selected from Column A, Rows 451, 272, 178, 111, 448, 412, 110 and 432 of Table 1 only when phosphorylated at the tyrosine listed in corresponding Column D of Table 1, comprised within the phosphorylatable peptide sequence listed in corresponding Column E of Table 1 (SEQ ID NOs: 450, 271, 177, 110, 447, 411, 109 and 431), wherein said antibody does not bind said signaling protein when not phosphorylated at said tyrosine.

54. An isolated phosphorylation site-specific antibody according to claim 17, that specifically binds a human Leukemia-related signaling protein selected from Column A, Rows 451, 272, 178, 111, 448, 412, 110 and 432 of Table 1 only when not phosphorylated at the tyrosine listed in corresponding Column D of Table 1, comprised within the phosphorylatable peptide sequence listed in corresponding Column E of Table 1 (SEQ ID NOs: SEQ ID NOs: 450, 271, 177, 110, 447, 411, 109 and 431), wherein said antibody does not bind said signaling protein when phosphorylated at said tyrosine.

55. A method selected from the group consisting of:
(a) a method for detecting a human leukemia-related signaling protein selected from Column A of Table 1, wherein said human leukemia-related signaling protein is phosphorylated at the tyrosine listed in corresponding Column D of Table 1, comprised within the phosphorylatable peptide sequence listed in corresponding Column E of Table 1 (SEQ ID NOs: 1-7, 9-14, 16, 19-21, 23, 26-30, 32-34, 36-45, 48-52, 56-58, 60-90, 93-119, 121-124, 129-151, 153-160, 163-180, 182-193, 195-197, 199-208, 210-221, 223-279, 281-294, 296-297, 299-316, 319-336, 339-345, 347-356, 358, 360-366, 368-378, 380-417, 419-438, 440-474, and 476-480), comprising the step of adding an isolated phosphorylation-specific antibody according to claim 16, to a sample comprising said human leukemia-related signaling protein under conditions that permit the binding of said antibody to said human leukemia-related signaling protein, and detecting bound antibody;
(b) a method for quantifying the amount of a human leukemia-related signaling protein listed in Column A of Table 1 that is phosphorylated at the corresponding tyrosine listed in Column D of Table 1, comprised within the phosphorylatable peptide sequence listed in corresponding Column E of Table 1 (SEQ ID NOs: 1-7, 9-14, 16, 19-21, 23, 26-30, 32-34, 36-45, 48-52, 56-58, 60-90, 93-119, 121-124, 129-151, 153-160, 163-180, 182-193, 195-197, 199-208, 210-221, 223-279, 281-294, 296-297, 299-316, 319-336, 339-345, 347-356, 358, 360-366, 368-378, 380-417, 419-438, 440-474, and 476-480), in a sample using a heavy-isotope labeled peptide (AQUA™ peptide), said labeled peptide comprising a phosphorylated tyrosine at said corresponding tyrosine listed Column D of Table 1, comprised within the phosphorylatable peptide sequence listed in corresponding Column E of Table 1 as an internal standard; and
(c) a method comprising step (a) followed by step (b).

56. The method of claim 55, wherein said isolated phosphorylation-specific antibody is capable of specifically binding GNAI2 only when phosphorylated at Y61, comprised within the phosphorylatable peptide sequence listed in Column E, Row 178, of Table 1 (SEQ ID NO: 177), wherein said antibody does not bind said protein when not phosphorylated at said tyrosine.

57. The method of claim 55, wherein said isolated phosphorylation-specific antibody is capable of specifically binding GNAI2 only when not phosphorylated at Y61, comprised within the phosphorylatable peptide sequence listed in Column E, Row 178, of Table 1 (SEQ ID NO: 177), wherein said antibody does not bind said protein when phosphorylated at said tyrosine.

58. The method of claim 55, wherein said isolated phosphorylation-specific antibody is capable of specifically binding GSTP1 only when phosphorylated at Y8, comprised within the phosphorylatable peptide sequence listed in Column E, Row 432, of Table 1 (SEQ ID NO: 431), wherein said antibody does not bind said protein when not phosphorylated at said tyrosine.

59. The method of claim 55, wherein said isolated phosphorylation-specific antibody is capable of specifically binding GSTP1 only when not phosphorylated at Y8, comprised within the phosphorylatable peptide sequence listed in Column E, Row 432, of Table 1 (SEQ ID NO: 431), wherein said antibody does not bind said protein when phosphorylated at said tyrosine.

60. The method of claim 55, wherein said isolated phosphorylation-specific antibody is capable of specifically binding eEF1A-1 only when phosphorylated at Y86, comprised within the phosphorylatable peptide sequence listed in Column E, Row 451, of Table 1 (SEQ ID NO: 450), wherein said antibody does not bind said protein when not phosphorylated at said tyrosine.

61. The method of claim 55, wherein said isolated phosphorylation-specific antibody is capable of specifically binding eEF1A-1 only when not phosphorylated at Y86, comprised within the phosphorylatable peptide sequence listed in Column E, Row 451, of Table 1 (SEQ ID NO: 450), wherein said antibody does not bind said protein when phosphorylated at said tyrosine.

62. The method of claim 55, wherein said isolated phosphorylation-specific antibody is capable of specifically binding eIF4G2 only when phosphorylated at Y439, comprised within the phosphorylatable peptide sequence listed in Column E, Row 448, of Table 1 (SEQ ID NO: 447), wherein said antibody does not bind said protein when not phosphorylated at said tyrosine.

63. The method of claim 55, wherein said isolated phosphorylation-specific antibody is capable of specifically binding eIF4G2 only when not phosphorylated at Y439, comprised within the phosphorylatable peptide sequence listed in Column E, Row 448, of Table 1 (SEQ ID NO: 447), wherein said antibody does not bind said protein when phosphorylated at said tyrosine.

64. The method of claim 55, wherein said isolated phosphorylation-specific antibody is capable of specifically binding eIF4G2 only when phosphorylated at Y439, comprised within the phosphorylatable peptide sequence listed in Column E, Row 448, of Table 1 (SEQ ID NO: 447), wherein said antibody does not bind said protein when not phosphorylated at said tyrosine.

65. The method of claim 55, wherein said isolated phosphorylation-specific antibody is capable of specifically binding eIF4G2 only when not phosphorylated at Y439, comprised within the phosphorylatable peptide sequence listed in Column E, Row 448, of Table 1 (SEQ ID NO: 447), wherein said antibody does not bind said protein when phosphorylated at said tyrosine.

66. The method of claim 55, wherein said isolated phosphorylation-specific antibody is capable of specifically binding Btk only when phosphorylated at Y344, comprised within the phosphorylatable peptide sequence listed in Column E, Row 272, of Table 1 (SEQ ID NO: 271), wherein said antibody does not bind said protein when not phosphorylated at said tyrosine.

67. The method of claim 55, wherein said isolated phosphorylation-specific antibody is capable of specifically binding Btk only when not phosphorylated at Y344, comprised within the phosphorylatable peptide sequence listed in Column E, Row 272, of Table 1 (SEQ ID NO: 271), wherein said antibody does not bind said protein when phosphorylated at said tyrosine.

68. The method of claim 55, wherein said isolated phosphorylation-specific antibody is capable of specifically binding HSPCA only when phosphorylated at Y641, comprised within the phosphorylatable peptide sequence listed in Column E, Row 111, of Table 1 (SEQ ID NO: 110), wherein said antibody does not bind said protein when not phosphorylated at said tyrosine.

69. The method of claim 55, wherein said isolated phosphorylation-specific antibody is capable of specifically binding HSPCA only when not phosphorylated at Y641, comprised within the phosphorylatable peptide sequence listed in Column E, Row 111, of Table 1 (SEQ ID NO: 110), wherein said antibody does not bind said protein when phosphorylated at said tyrosine.

70. The method of claim 55, wherein said isolated phosphorylation-specific antibody is capable of specifically binding STAT5A only when phosphorylated at Y90, comprised within the phosphorylatable peptide sequence listed in Column E, Row 412, of Table 1 (SEQ ID NO: 411), wherein said antibody does not bind said protein when not phosphorylated at said tyrosine.

71. The method of claim 55, wherein said isolated phosphorylation-specific antibody is capable of specifically binding STAT5A only when not phosphorylated at Y90, comprised within the phosphorylatable peptide sequence listed in Column E, Row 412, of Table 1 (SEQ ID NO: 411), wherein said antibody does not bind said protein when phosphorylated at said tyrosine.

72. The method of claim 55, wherein said isolated phosphorylation-specific antibody is capable of specifically binding HSPCA only when phosphorylated at Y3 19, comprised within the phosphorylatable peptide sequence listed in Column E, Row 110, of Table 1 (SEQ ID NO: 109), wherein said antibody does not bind said protein when not phosphorylated at said tyrosine.

73. The method of claim 55, wherein said isolated phosphorylation-specific antibody is capable of specifically binding HSPCA only when not phosphorylated at Y3 19, comprised within the phosphorylatable peptide sequence listed in Column E, Row 272, of Table 1 (SEQ ID NO: 271), wherein said antibody does not bind said protein when phosphorylated at said tyrosine.


SEQUENCE LISTING (480 sequences)

SEQ ID NO: 1 - protein, 36 aa, Homo sapiens
  MOD_RES (4)..(4): PHOSPHORYLATION; tyrosine at position 4 is phosphorylated
GLMYGGVWATVQFLSKSFFDHSMHSALPLGIYLATK

SEQ ID NO: 2 - protein, 36 aa, Homo sapiens; identical to SEQ ID NO: 1
  MOD_RES (32)..(32): PHOSPHORYLATION; tyrosine at position 32 is phosphorylated

SEQ ID NO: 3 - protein, 14 aa, Homo sapiens
  MOD_RES (10)..(10): PHOSPHORYLATION; tyrosine at position 10 is phosphorylated
CASQSGMTAYGTRR

SEQ ID NO: 4 - protein, 27 aa, Homo sapiens
  MOD_RES (9)..(9): PHOSPHORYLATION; tyrosine at position 9 is phosphorylated
EGTQASEGYFSQSQEEEFAQSEELCAK

SEQ ID NO: 5 - protein, 13 aa, Homo sapiens
  MOD_RES (9)..(9): PHOSPHORYLATION; tyrosine at position 9 is phosphorylated
VAKASGANYSFHK

SEQ ID NO: 6 - protein, 19 aa, Homo sapiens
  MOD_RES (1)..(1): PHOSPHORYLATION; tyrosine at position 1 is phosphorylated
YVCMKKNGQLAAISDFVGK

SEQ ID NO: 7 - protein, 37 aa, Homo sapiens
  MOD_RES (37)..(37): PHOSPHORYLATION; tyrosine at position 37 is phosphorylated
DIYTAGAGVGDIGVEVEDPQGKNTVELLVEDKGNQVY

SEQ ID NO: 8 - protein, 15 aa, Homo sapiens
  MOD_RES (8)..(8): PHOSPHORYLATION; tyrosine at position 8 is phosphorylated
VDTDGNGYISFNELN

SEQ ID NO: 9 - protein, 27 aa, Homo sapiens
  MOD_RES (2)..(2): PHOSPHORYLATION; tyrosine at position 4 is phosphorylated
TYLISSIPLQGAFNYKYTACLCDDNPK

SEQ ID NO: 10 - protein, 25 aa, Homo sapiens
  MOD_RES (4)..(4): PHOSPHORYLATION; tyrosine at position 4 is phosphorylated
HSPYRTLEPVRPPVVPNDYVPSPTR

SEQ ID NO: 11 - protein, 18 aa, Homo sapiens
  MOD_RES (14)..(14): PHOSPHORYLATION; tyrosine at position 4 is phosphorylated
STQPQQNNEELPTYEEAK

SEQ ID NO: 12 - protein, 34 aa, Homo sapiens
  MOD_RES (11)..(11): PHOSPHORYLATION; tyrosine at position 11 is phosphorylated
EGQNANMENLYTALQSIDRGEIVNMLEGSGRQSR

SEQ ID NO: 13 - protein, 14 aa, Homo sapiens
  MOD_RES (9)..(9): PHOSPHORYLATION; tyrosine at position 9  is phosphorylated
LKGMKDDDYDDQLC

SEQ ID NO: 14 - protein, 15 aa, Homo sapiens
  MOD_RES (8)..(8): PHOSPHORYLATION; tyrosine at position 8 is phosphorylated
DRPQSSIYDPFAGMK

SEQ ID NO: 15 - protein, 15 aa, Homo sapiens
  MOD_RES (8)..(8): PHOSPHORYLATION; tyrosine at position 8 is phosphorylated
AKVEFGVYESGPRKS

SEQ ID NO: 16 - protein, 26 aa, Homo sapiens
  MOD_RES (1)..(1): PHOSPHORYLATION; tyrosine at position 8 is phosphorylated
YQKDVYDIPPSHTTQGVYDIPPSSAK

SEQ ID NO: 17 - protein, 26 aa, Homo sapiens
  MOD_RES (6)..(6): PHOSPHORYLATION; tyrosine at position 6 is phosphorylated
YQKDVYDIPPSHTTQGVYDIPPSSAK

SEQ ID NO: 18 - protein, 23 aa, Homo sapiens
  MOD_RES (15)..(15): PHOSPHORYLATION; tyrosine at position 15 is phosphorylated
DVYDIPPSHTTQGVYDIPPSSAK

SEQ ID NO: 19 - protein, 12 aa, Homo sapiens
  MOD_RES (1)..(1): PHOSPHORYLATION; tyrosine at position 1 is phosphorylated
YFSLKPEEKDEK

SEQ ID NO: 20 - protein, 11 aa, Homo sapiens
  MOD_RES (3)..(3): PHOSPHORYLATION; tyrosine at position 3 is phosphorylated
NDYEARPQYYK

SEQ ID NO: 21 - protein, 20 aa, Homo sapiens
  MOD_RES (16)..(16): PHOSPHORYLATION; tyrosine at position 16 is phosphorylated
KGEQTSSGTLSAFASYFNSK

SEQ ID NO: 22 - protein, 15 aa, Homo sapiens
  MOD_RES (8)..(8): PHOSPHORYLATION; tyrosine at position 8 is phosphorylated
CMEENELYSSAVTVG

SEQ ID NO: 23 - protein, 32 aa, Homo sapiens
  MOD_RES (11)..(11): PHOSPHORYLATION; tyrosine at position 11 is phosphorylated
VPPQLLADPLYDSIEETLPPRPDHIYDEPEGV

SEQ ID NO: 24 - protein, 26 aa, Homo sapiens
  MOD_RES (20)..(20): PHOSPHORYLATION; tyrosine at position 20 is phosphorylated
ADPLYDSIEETLPPRPDHIYDEPEGV

SEQ ID NO: 25 - protein, 33 aa, Homo sapiens
  MOD_RES (9)..(9): PHOSPHORYLATION; tyrosine at position 9 is phosphorylated
AGDNSQSVYIPMSPGAHHFDSLGYPSTTLPVHR

SEQ ID NO: 26 - protein, 14 aa, Homo sapiens
  MOD_RES (12)..(12): PHOSPHORYLATION; tyrosine at position 12 is phosphorylated
ANHTFNSSSSQYCR

SEQ ID NO: 27 - protein, 15 aa, Homo sapiens
  MOD_RES (9)..(9): PHOSPHORYLATION; tyrosine at position 4 is phosphorylated
LSLPEIPSYGFLVPR

SEQ ID NO: 28 - protein, 11 aa, Homo sapiens
  MOD_RES (10)..(10): PHOSPHORYLATION; tyrosine at position 10 is phosphorylated
GLQVEEEPVYE

SEQ ID NO: 29 - protein, 14 aa, Homo sapiens
  MOD_RES (11)..(11): PHOSPHORYLATION; tyrosine at position 11 is phosphorylated
SSKYPEDDPDYCVW

SEQ ID NO: 30 - protein, 9 aa, Homo sapiens
  MOD_RES (6)..(6): PHOSPHORYLATION; tyrosine at position 6 is phosphorylated
RSEKIYQQR

SEQ ID NO: 31 - protein, 15 aa, Homo sapiens
  MOD_RES (8)..(8): PHOSPHORYLATION; tyrosine at position 8 is phosphorylated
SFTGSRTYSLVGQAW

SEQ ID NO: 32 - protein, 15 aa, Homo sapiens
  MOD_RES (5)..(5): PHOSPHORYLATION; tyrosine at position 5 is phosphorylated
LLQFYPSLEDPASSR

SEQ ID NO: 33 - protein, 15 aa, Homo sapiens
  MOD_RES (1)..(1): PHOSPHORYLATION; tyrosine at position 1 is phosphorylated
YTEFYHVPTHSDASK

SEQ ID NO: 34 - protein, 16 aa, Homo sapiens
  MOD_RES (14)..(14): PHOSPHORYLATION; tyrosine at position 14 is phosphorylated
FVEFDMKPVCKKCYEK

SEQ ID NO: 35 - protein, 15 aa, Homo sapiens
  MOD_RES (8)..(8): PHOSPHORYLATION; tyrosine at position 8 is phosphorylated
YDERGELYDDVDHPL

SEQ ID NO: 36 - protein, 14 aa, Homo sapiens
  MOD_RES (7)..(7): PHOSPHORYLATION; tyrosine at position 7 is phosphorylated
LDDDGPYSGPFCGR

SEQ ID NO: 37 - protein, 19 aa, Homo sapiens
  MOD_RES (8)..(8): PHOSPHORYLATION; tyrosine at position 8 is phosphorylated
AAEEVMCYTSLQLRPPQGR

SEQ ID NO: 38 - protein, 19 aa, Homo sapiens
  MOD_RES (11)..(11): PHOSPHORYLATION; tyrosine at position 11 is phosphorylated
SQASGPEPELYASVCAQTR

SEQ ID NO: 39 - protein, 13 aa, Homo sapiens
  MOD_RES (8)..(8): PHOSPHORYLATION; tyrosine at position 8 is phosphorylated
LMNEDPMYSMYAK

SEQ ID NO: 40 - protein, 13 aa, Homo sapiens
  MOD_RES (11)..(11): PHOSPHORYLATION; tyrosine at position 11 is phosphorylated
LMNEDPMYSMYAK

SEQ ID NO: 41 - protein, 10 aa, Homo sapiens
  MOD_RES (5)..(5): PHOSPHORYLATION; tyrosine at position 5 is phosphorylated
GLFYYYANEK

SEQ ID NO: 42 - protein, 29 aa, Homo sapiens
  MOD_RES (26)..(26): PHOSPHORYLATION; tyrosine at position 26 is phosphorylated
YGYVLRSYLADNDGEIYDDIADGCIYDND

SEQ ID NO: 43 - protein, 19 aa, Homo sapiens
  MOD_RES (6)..(6): PHOSPHORYLATION; tyrosine at position 6 is phosphorylated
PSSINYMVAPVTGNDVGIR

SEQ ID NO: 44 - protein, 41 aa, Homo sapiens
  MOD_RES (17)..(17): PHOSPHORYLATION; tyrosine at position 17 is phosphorylated
TGSLKPNPASPLPASPYGGPTPASYTTASTPAGPAFPVQVK

SEQ ID NO: 45 - protein, 41 aa, Homo sapiens; identical to SEQ ID NO: 44
  MOD_RES (25)..(25): PHOSPHORYLATION; tyrosine at position 25 is phosphorylated

SEQ ID NO: 46 - protein, 15 aa, Homo sapiens
  MOD_RES (8)..(8): PHOSPHORYLATION; tyrosine at position 8 is phosphorylated
PEERRHQYSDYDYHS

SEQ ID NO: 47 - protein, 15 aa, Homo sapiens
  MOD_RES (8)..(8): PHOSPHORYLATION; tyrosine at position 8 is phosphorylated
HQYSDYDYHSSSEKL

SEQ ID NO: 48 - protein, 16 aa, Homo sapiens
  MOD_RES (12)..(12): PHOSPHORYLATION; tyrosine at position 12 is phosphorylated
EFVFDTIQSHQYQCPR

SEQ ID NO: 49 - protein, 15 aa, Homo sapiens
  MOD_RES (8)..(8): PHOSPHORYLATION; tyrosine at position 8 is phosphorylated
TAEDNLEYVRTLYDF

SEQ ID NO: 50 - protein, 26 aa, Homo sapiens
  MOD_RES (3)..(3): PHOSPHORYLATION; tyrosine at position 3 is phosphorylated
LLYMDRKEARVCVICHSVLMNVAQPR

SEQ ID NO: 51 - protein, 19 aa, Homo sapiens
  MOD_RES (14)..(14): PHOSPHORYLATION; tyrosine at position 14 is phosphorylated
LSYLSAPGSEYSMYSTDSR

SEQ ID NO: 52 - protein, 8 aa, Homo sapiens
  MOD_RES (2)..(2): PHOSPHORYLATION; tyrosine at position 2 is phosphorylated
GYYGQSAR

SEQ ID NO: 53 - protein, 15 aa, Homo sapiens
  MOD_RES (8)..(8): PHOSPHORYLATION; tyrosine at position 8 is phosphorylated
PLPAQLVYTTNIQEL

SEQ ID NO: 54 - protein, 15 aa, Homo sapiens
  MOD_RES (8)..(8): PHOSPHORYLATION; tyrosine at position 8 is phosphorylated
NIQELNVYSEAQEPK

SEQ ID NO: 55 - protein, 15 aa, Homo sapiens
  MOD_RES (8)..(8): PHOSPHORYLATION; tyrosine at position 8is phosphorylated
SAQPSPHYMAAPSSG

SEQ ID NO: 56 - protein, 25 aa, Homo sapiens
  MOD_RES (19)..(19): PHOSPHORYLATION; tyrosine at position 19 is phosphorylated
REPGYTPPGAGNQNPPGMYPVTGPK

SEQ ID NO: 57 - protein, 14 aa, Homo sapiens
  MOD_RES (8)..(8): PHOSPHORYLATION; tyrosine at position 8 is phosphorylated
QENANSGYYEAHPV

SEQ ID NO: 58 - protein, 14 aa, Homo sapiens
  MOD_RES (9)..(9): PHOSPHORYLATION; tyrosine at position 9 is phosphorylated
QENANSGYYEAHPV

SEQ ID NO: 59 - protein, 15 aa, Homo sapiens
  MOD_RES (8)..(8): PHOSPHORYLATION; tyrosine at position 8 is phosphorylated
VSERKMIYIITIDGQ

SEQ ID NO: 60 - protein, 16 aa, Homo sapiens
  MOD_RES (3)..(3): PHOSPHORYLATION; tyrosine at position 3 is phosphorylated
ALYDYMGSEMMAVTQK

SEQ ID NO: 61 - protein, 26 aa, Homo sapiens
  MOD_RES (6)..(6): PHOSPHORYLATION; tyrosine at position 6 is phosphorylated
LAAEVYKDMPETSFTRTISNPEVVMK

SEQ ID NO: 62 - protein, 27 aa, Homo sapiens
  MOD_RES (21)..(21): PHOSPHORYLATION; tyrosine at position 21 is phosphorylated
NPQELYDVYCFARELGGEVFYVGPARR

SEQ ID NO: 63 - protein, 25 aa, Homo sapiens
  MOD_RES (2)..(2): PHOSPHORYLATION; tyrosine at position 2 is phosphorylated
VYLRVPSHLQAETLVGKVNLEECLK

SEQ ID NO: 64 - protein, 23 aa, Homo sapiens
  MOD_RES (14)..(14): PHOSPHORYLATION; tyrosine at position 14 is phosphorylated
ISQMITSEGNVVSYLNISSSQVR

SEQ ID NO: 65 - protein, 25 aa, Homo sapiens
  MOD_RES (14)..(14): PHOSPHORYLATION; tyrosine at position 14 is phosphorylated
IGSPEYIKSYKIAYSNDGKTWAMYK

SEQ ID NO: 66 - protein, 25 aa, Homo sapiens
  MOD_RES (24)..(24): PHOSPHORYLATION; tyrosine at position 24 is phosphorylated
IGSPEYIKSYKIAYSNDGKTWAMYK

SEQ ID NO: 67 - protein, 29 aa, Homo sapiens
  MOD_RES (18)..(18): PHOSPHORYLATION
GPTSGPQSAPQIYGPPQYNIQYSSSAAVK

SEQ ID NO: 68 - protein, 15 aa, Homo sapiens
  MOD_RES (3)..(3): PHOSPHORYLATION; tyrosine at position 3 is phosphorylated
RQYADCSEIFNDGYK

SEQ ID NO: 69 - protein, 17 aa, Homo sapiens
  MOD_RES (14)..(14): PHOSPHORYLATION; tyrosine at position 14 is phosphorylated
EFKVAVNGVHSLEYKHR

SEQ ID NO: 70 - protein, 12 aa, Homo sapiens
  MOD_RES (4)..(4): PHOSPHORYLATION; tyrosine at position 4 is phosphorylated
MGTYGVRAAWRR

SEQ ID NO: 71 - protein, 18 aa, Homo sapiens
  MOD_RES (11)..(11): PHOSPHORYLATION; tyrosine at position 11 is phosphorylated
FPMADGHTDSYSTSAVLR

SEQ ID NO: 72 - protein, 19 aa, Homo sapiens
  MOD_RES (12)..(12): PHOSPHORYLATION; tyrosine at position 12 is phosphorylated
MSDPNMEANSHYGHNDDVR

SEQ ID NO: 73 - protein, 19 aa, Homo sapiens
  MOD_RES (19)..(19): PHOSPHORYLATION; tyrosine at position 19 is phosphorylated
SDHPAEAGPISEDEQELHY

SEQ ID NO: 74 - protein, 17 aa, Homo sapiens
  MOD_RES (13)..(13): PHOSPHORYLATION; tyrosine at position 13 is phosphorylated
VQPQEPKVTDTEYSEIK

SEQ ID NO: 75 - protein, 31 aa, Homo sapiens
  MOD_RES (24)..(24): PHOSPHORYLATION; tyrosine at position 24 is phosphorylated
TVAEHVEAIEGSVQESSGPVTTIYMLAGKPR

SEQ ID NO: 76 - protein, 18 aa, Homo sapiens
  MOD_RES (15)..(15): PHOSPHORYLATION; tyrosine at position 15 is phosphorylated
LSHRPKTGFIRVVMYEGK

SEQ ID NO: 77 - protein, 29 aa, Homo sapiens
  MOD_RES (23)..(23): PHOSPHORYLATION; tyrosine at position 23 is phosphorylated
TGSGGPGNHPHGPDASAEGLNPYGLVAPR

SEQ ID NO: 78 - protein, 25 aa, Homo sapiens
  MOD_RES (5)..(5): PHOSPHORYLATION; tyrosine at position 5 is phosphorylated
EESTYLPLTSMQPTEAMGEEPSRAE

SEQ ID NO: 79 - protein, 27 aa, Homo sapiens
  MOD_RES (10)..(10): PHOSPHORYLATION; tyrosine at position 10 is phosphorylated
DGTIVDDDDYFLCLPSNTKFVALASNE

SEQ ID NO: 80 - protein, 12 aa, Homo sapiens
  MOD_RES (12)..(12): PHOSPHORYLATION; tyrosine at position 12 is phosphorylated
RKVMDSDEDDDY

SEQ ID NO: 81 - protein, 24 aa, Homo sapiens
  MOD_RES (17)..(17): PHOSPHORYLATION; tyrosine at position 17 is phosphorylated
EEEEEEEEEEEEASTEYGHQAHRH

SEQ ID NO: 82 - protein, 14 aa, Homo sapiens
  MOD_RES (7)..(7): PHOSPHORYLATION; tyrosine at position 7 is phosphorylated
SNLDKLYHGLELAK

SEQ ID NO: 83 - protein, 25 aa, Homo sapiens
  MOD_RES (6)..(6): PHOSPHORYLATION; tyrosine at position 6 is phosphorylated
LLGSGYGGLTGGSSRGPPVTPSSEK

SEQ ID NO: 84 - protein, 15 aa, Homo sapiens
  MOD_RES (11)..(11): PHOSPHORYLATION; tyrosine at position 11 is phosphorylated
ALEQKPDDAQYYCQR

SEQ ID NO: 85 - protein, 21 aa, Homo sapiens
  MOD_RES (9)..(9): PHOSPHORYLATION; tyrosine at position 9 is phosphorylated
IIDKEGDDYEVIPNSNFYVSR

SEQ ID NO: 86 - protein, 19 aa, Homo sapiens
  MOD_RES (7)..(7): PHOSPHORYLATION; tyrosine at position 7 is phosphorylated
SNPTDIYPSKWIARLRHIK

SEQ ID NO: 87 - protein, 28 aa, Homo sapiens
  MOD_RES (22)..(22): PHOSPHORYLATION; tyrosine at position 22 is phosphorylated
TGDAESSEMQRPPPDCDDTVTYSALHKR

SEQ ID NO: 88 - protein, 18 aa, Homo sapiens
  MOD_RES (14)..(14): PHOSPHORYLATION; tyrosine at position 14 is phosphorylated
TPVSQKEESSATIYCSIR

SEQ ID NO: 89 - protein, 38 aa, Homo sapiens
  MOD_RES (14)..(14): PHOSPHORYLATION; tyrosine at position 14 is phosphorylated
LGQDPGADDDGEITYENVQVPAVLGVPSSLASSVLGDK

SEQ ID NO: 90 - protein, 19 aa, Homo sapiens
  MOD_RES (13)..(13): PHOSPHORYLATION; tyrosine at position 13 is phosphorylated
MQNHGYENPTYKYLEQMQI

SEQ ID NO: 91 - protein, 15 aa, Homo sapiens
  MOD_RES (8)..(8): PHOSPHORYLATION; tyrosine at position 8 is phosphorylated
AASKKTIYTYIMASR

SEQ ID NO: 92 - protein, 15 aa, Homo sapiens
  MOD_RES (8)..(8): PHOSPHORYLATION; tyrosine at position 8 is phosphorylated
QPAESRIYDEILQSK

SEQ ID NO: 93 - protein, 24 aa, Homo sapiens
  MOD_RES (13)..(13): PHOSPHORYLATION; tyrosine at position 13 is phosphorylated
VLPSKEEPVNTVYSEVQFADKMGK

SEQ ID NO: 94 - protein, 18 aa, Homo sapiens
  MOD_RES (6)..(6): PHOSPHORYLATION; tyrosine at position 6 is phosphorylated
HLEEDYADAYQDLYQPHR

SEQ ID NO: 95 - protein, 18 aa, Homo sapiens
  MOD_RES (10)..(10): PHOSPHORYLATION; tyrosine at position 10 is phosphorylated
HLEEDYADAYQDLYQPHR

SEQ ID NO: 96 - protein, 18 aa, Homo sapiens
  MOD_RES (14)..(14): PHOSPHORYLATION; tyrosine at position 14 is phosphorylated
HLEEDYADAYQDLYQPHR

SEQ ID NO: 97 - protein, 18 aa, Homo sapiens
  MOD_RES (9)..(9): PHOSPHORYLATION; tyrosine at position 9 is phosphorylated
HFTVVGDDYHAWNINYKK

SEQ ID NO: 98 - protein, 20 aa, Homo sapiens
  MOD_RES (19)..(19): PHOSPHORYLATION; tyrosine at position 19 is phosphorylated
FLRHFTVVGDDYHAWNINYK

SEQ ID NO: 99 - protein, 20 aa, Homo sapiens
  MOD_RES (9)..(9): PHOSPHORYLATION; tyrosine at position 9 is phosphorylated
ILDRLLDGYDNRLRPGLGER

SEQ ID NO: 100 - protein, 30 aa, Homo sapiens
  MOD_RES (22)..(22): PHOSPHORYLATION; tyrosine at position 22 is phosphorylated
NQYDLLGQTVDSGIVQSSTGEYVVMTTHFH

SEQ ID NO: 101 - protein, 23 aa, Homo sapiens
  MOD_RES (5)..(5): PHOSPHORYLATION; tyrosine at position 5 is phosphorylated
NLDPYEQWSDQEIWKVADEVGLR

SEQ ID NO: 102 - protein, 16 aa, Homo sapiens
  MOD_RES (7)..(7): PHOSPHORYLATION; tyrosine at position 7 is phosphorylated
TTNVSLYPGYNFRYAK

SEQ ID NO: 103 - protein, 16 aa, Homo sapiens
  MOD_RES (14)..(14): PHOSPHORYLATION; tyrosine at position 14 is phosphorylated
TTNVSLYPGYNFRYAK

SEQ ID NO: 104 - protein, 21 aa, Homo sapiens
  MOD_RES (14)..(14): PHOSPHORYLATION; tyrosine at position 14 is phosphorylated
EAERAKEVRGSGSYEYPVAEK

SEQ ID NO: 105 - protein, 21 aa, Homo sapiens
  MOD_RES (16)..(16): PHOSPHORYLATION; tyrosine at position 16 is phosphorylated
EAERAKEVRGSGSYEYPVAEK

SEQ ID NO: 106 - protein, 15 aa, Homo sapiens
  MOD_RES (8)..(8): PHOSPHORYLATION; tyrosine at position 8 is phosphorylated
FINRQLIYNYPEQLF

SEQ ID NO: 107 - protein, 18 aa, Homo sapiens
  MOD_RES (10)..(10): PHOSPHORYLATION; tyrosine at position 10 is phosphorylated
MLNSNTESPYLIWNNSTR

SEQ ID NO: 108 - protein, 8 aa, Homo sapiens
  MOD_RES (7)..(7): PHOSPHORYLATION; tyrosine at position 7 is phosphorylated
STETALYR

SEQ ID NO: 109 - protein, 16 aa, Homo sapiens
  MOD_RES (12)..(12): PHOSPHORYLATION; tyrosine at position 12 is phosphorylated
VILHLKEDQTEYLEER

SEQ ID NO: 110 - protein, 11 aa, Homo sapiens
  MOD_RES (6)..(6): PHOSPHORYLATION; tyrosine at position 6 is phosphorylated
NQKHIYYITGE

SEQ ID NO: 111 - protein, 10 aa, Homo sapiens
  MOD_RES (5)..(5): PHOSPHORYLATION; tyrosine at position 5 is phosphorylated
GNFNYIEFTR

SEQ ID NO: 112 - protein, 12 aa, Homo sapiens
  MOD_RES (6)..(6): PHOSPHORYLATION; tyrosine at position 6 is phosphorylated
TVAAYYEEQHPE

SEQ ID NO: 113 - protein, 23 aa, Homo sapiens
  MOD_RES (18)..(18): PHOSPHORYLATION; tyrosine at position 18 is phosphorylated
KDDPVTNLNNAFEVAEKYLDIPK

SEQ ID NO: 114 - protein, 16 aa, Homo sapiens
  MOD_RES (12)..(12): PHOSPHORYLATION; tyrosine at position 12 is phosphorylated
YFDRVDENNPEYLRER

SEQ ID NO: 115 - protein, 10 aa, Homo sapiens
  MOD_RES (8)..(8): PHOSPHORYLATION; tyrosine at position 8 is phosphorylated
WLNSPNTYMK

SEQ ID NO: 116 - protein, 28 aa, Homo sapiens
  MOD_RES (20)..(20): PHOSPHORYLATION; tyrosine at position 20 is phosphorylated
RQTALDNEKDRDSHEDGDYYEVDINGPE

SEQ ID NO: 117 - protein, 15 aa, Homo sapiens
  MOD_RES (6)..(6): PHOSPHORYLATION; tyrosine at position 6 is phosphorylated
DVDAAYMNKVELQAK

SEQ ID NO: 118 - protein, 18 aa, Homo sapiens
  MOD_RES (4)..(4): PHOSPHORYLATION; tyrosine at position 4 is phosphorylated
SRDYNPYNYSDSISPFNK

SEQ ID NO: 119 - protein, 18 aa, Homo sapiens
  MOD_RES (9)..(9): PHOSPHORYLATION; tyrosine at position 9 is phosphorylated
SRDYNPYNYSDSISPFNK

SEQ ID NO: 120 - protein, 15 aa, Homo sapiens
  MOD_RES (8)..(8): PHOSPHORYLATION; tyrosine at position 8 is phosphorylated
GQTVDDPYATFVKML

SEQ ID NO: 121 - protein, 16 aa, Homo sapiens
  MOD_RES (12)..(12): PHOSPHORYLATION; tyrosine at position 12 is phosphorylated
TLGPEVHPDTIYSVDW

SEQ ID NO: 122 - protein, 29 aa, Homo sapiens
  MOD_RES (10)..(10): PHOSPHORYLATION; tyrosine at position 10 is phosphorylated
KSDLFQEDLYPPTAGPDPALTAEEWLGGR

SEQ ID NO: 123 - protein, 10 aa, Homo sapiens
  MOD_RES (9)..(9): PHOSPHORYLATION; tyrosine at position 9 is phosphorylated
ISEDKNPDYR

SEQ ID NO: 124 - protein, 25 aa, Homo sapiens
  MOD_RES (12)..(12): PHOSPHORYLATION; tyrosine at position 12 is phosphorylated
KEDALLYQSKGYNDDYYEESYFTTR

SEQ ID NO: 125 - protein, 15 aa, Homo sapiens
  MOD_RES (8)..(8): PHOSPHORYLATION; tyrosine at position 8 is phosphorylated
KDRERPMYGRDSAYQ

SEQ ID NO: 126 - protein, 15 aa, Homo sapiens
  MOD_RES (8)..(8): PHOSPHORYLATION; tyrosine at position 8 is phosphorylated
MYGRDSAYQSITHYR

SEQ ID NO: 127 - protein, 15 aa, Homo sapiens
  MOD_RES (8)..(8): PHOSPHORYLATION; tyrosine at position 8 is phosphorylated
RSSLDLSYYPTSSST

SEQ ID NO: 128 - protein, 12 aa, Homo sapiens
  MOD_RES (5)..(5): PHOSPHORYLATION; tyrosine at position 5 is phosphorylated
EPSNYDFVYDCN

SEQ ID NO: 129 - protein, 12 aa, Homo sapiens
  MOD_RES (9)..(9): PHOSPHORYLATION; tyrosine at position 9 is phosphorylated
EPSNYDFVYDCN

SEQ ID NO: 130 - protein, 21 aa, Homo sapiens
  MOD_RES (1)..(1): PHOSPHORYLATION; tyrosine at position 1 is phosphorylated
YNVPLNRLKMMFEKGEPTQTK

SEQ ID NO: 131 - protein, 11 aa, Homo sapiens
  MOD_RES (3)..(3): PHOSPHORYLATION; tyrosine at position 3 is phosphorylated
NRYYDEDEDEE

SEQ ID NO: 132 - protein, 8 aa, Homo sapiens
  MOD_RES (2)..(2): PHOSPHORYLATION; tyrosine at position 2 is phosphorylated
SYTLVVAK

SEQ ID NO: 133 - protein, 25 aa, Homo sapiens
  MOD_RES (12)..(12): PHOSPHORYLATION; tyrosine at position 12 is phosphorylated
VTEGTIREEQEYEEEVEEEPRPAAK

SEQ ID NO: 134 - protein, 29 aa, Homo sapiens
  MOD_RES (22)..(22): PHOSPHORYLATION; tyrosine at position 22 is phosphorylated
LNDLEEALQQAKEDLARLLRDYQELMNVK

SEQ ID NO: 135 - protein, 26 aa, Homo sapiens
  MOD_RES (25)..(25): PHOSPHORYLATION; tyrosine at position 25 is phosphorylated
NTSEQDQPMGGWEMIRKIGDTSVSYK

SEQ ID NO: 136 - protein, 14 aa, Homo sapiens
  MOD_RES (10)..(10): PHOSPHORYLATION; tyrosine at position 10 is phosphorylated
VVEPKDTAIYQKDE

SEQ ID NO: 137 - protein, 20 aa, Homo sapiens
  MOD_RES (12)..(12): PHOSPHORYLATION; tyrosine at position 12 is phosphorylated
GREDVALEQDTYWRELSCER

SEQ ID NO: 138 - protein, 17 aa, Homo sapiens
  MOD_RES (7)..(7): PHOSPHORYLATION; tyrosine at position 7 is phosphorylated
TPGDFSYAYQKPEETTR

SEQ ID NO: 139 - protein, 31 aa, Homo sapiens
  MOD_RES (9)..(9): PHOSPHORYLATION; tyrosine at position 9 is phosphorylated
TPGDFSYAYQKPEETTRSPDEEDYDYESYEK

SEQ ID NO: 140 - protein, 14 aa, Homo sapiens
  MOD_RES (12)..(12): PHOSPHORYLATION; tyrosine at position 12 is phosphorylated
SPDEEDYDYESYEK

SEQ ID NO: 141 - protein, 11 aa, Homo sapiens
  MOD_RES (7)..(7): PHOSPHORYLATION; tyrosine at position 7 is phosphorylated
TSDVGGYYYEK

SEQ ID NO: 142 - protein, 11 aa, Homo sapiens
  MOD_RES (8)..(8): PHOSPHORYLATION; tyrosine at position 8 is phosphorylated
TSDVGGYYYEK

SEQ ID NO: 143 - protein, 14 aa, Homo sapiens
  MOD_RES (7)..(7): PHOSPHORYLATION; tyrosine at position 7 is phosphorylated
SPSDSGYSYETIGK

SEQ ID NO: 144 - protein, 14 aa, Homo sapiens
  MOD_RES (7)..(7): PHOSPHORYLATION; tyrosine at position 7 is phosphorylated
TPEEGGYSYDISEK

SEQ ID NO: 145 - protein, 14 aa, Homo sapiens
  MOD_RES (9)..(9): PHOSPHORYLATION; tyrosine at position 9 is phosphorylated
TPEEGGYSYDISEK

SEQ ID NO: 146 - protein, 14 aa, Homo sapiens
  MOD_RES (10)..(10): PHOSPHORYLATION; tyrosine at position 10 is phosphorylated
TTSPPEVSGYSYEK

SEQ ID NO: 147 - protein, 14 aa, Homo sapiens
  MOD_RES (12)..(12): PHOSPHORYLATION; tyrosine at position 12 is phosphorylated
TTSPPEVSGYSYEK

SEQ ID NO: 148 - protein, 31 aa, Homo sapiens
  MOD_RES (9)..(9): PHOSPHORYLATION; tyrosine at position 9 is phosphorylated
LLDDISNGYDDSEDGGHTLGDPSYSYETTEK

SEQ ID NO: 149 - protein, 31 aa, Homo sapiens; identical to SEQ ID NO: 148
  MOD_RES (24)..(24): PHOSPHORYLATION; tyrosine at position 24 is phosphorylated

SEQ ID NO: 150 - protein, 31 aa, Homo sapiens; identical to SEQ ID NO: 148
  MOD_RES (26)..(26): PHOSPHORYLATION; tyrosine at position 26 is phosphorylated

SEQ ID NO: 151 - protein, 17 aa, Homo sapiens
  MOD_RES (12)..(12): PHOSPHORYLATION; tyrosine at position 12is phosphorylated
ITSFPESEGYSYETSTK

SEQ ID NO: 152 - protein, 9 aa, Homo sapiens
  MOD_RES (1)..(1): PHOSPHORYLATION; tyrosine at position 1 is phosphorylated
YQPKGGQVR

SEQ ID NO: 153 - protein, 9 aa, Homo sapiens
  MOD_RES (3)..(3): PHOSPHORYLATION; tyrosine at position 3 is phosphorylated
VSYSHIQSK

SEQ ID NO: 154 - protein, 18 aa, Homo sapiens
  MOD_RES (10)..(10): PHOSPHORYLATION; tyrosine at position 10 is phosphorylated
AYELQSDNVYKADLEWLR

SEQ ID NO: 155 - protein, 20 aa, Homo sapiens
  MOD_RES (12)..(12): PHOSPHORYLATION; tyrosine at position 12 is phosphorylated
SRSSSVGSSSSYPISPAVSR

SEQ ID NO: 156 - protein, 17 aa, Homo sapiens
  MOD_RES (10)..(10): PHOSPHORYLATION; tyrosine at position 10 is phosphorylated
NDGSVAGVRYFQCEPLK

SEQ ID NO: 157 - protein, 8 aa, Homo sapiens
  MOD_RES (4)..(4): PHOSPHORYLATION; tyrosine at position 4 is phosphorylated
GPAYGLSR

SEQ ID NO: 158 - protein, 26 aa, Homo sapiens
  MOD_RES (12)..(12): PHOSPHORYLATION; tyrosine at position 12 is phosphorylated
TSSISGPLSPAYTGQVPYNYNQLEGR

SEQ ID NO: 159 - protein, 13 aa, Homo sapiens
  MOD_RES (9)..(9): PHOSPHORYLATION; tyrosine at position 9 is phosphorylated
IGITNHDEYSLVR

SEQ ID NO: 160 - protein, 17 aa, Homo sapiens
  MOD_RES (10)..(10): PHOSPHORYLATION; tyrosine at position 10 is phosphorylated
FTSGKYQDVYVELSHIK

SEQ ID NO: 161 - protein, 19 aa, Homo sapiens
  MOD_RES (6)..(6): PHOSPHORYLATION; tyrosine at position 6 is phosphorylated
DNEAIYDICRRNLDIERPT

SEQ ID NO: 162 - protein, 14 aa, Homo sapiens
  MOD_RES (9)..(9): PHOSPHORYLATION; tyrosine at position 9 is phosphorylated
NLDIERPTYTNLNR

SEQ ID NO: 163 - protein, 24 aa, Homo sapiens
  MOD_RES (2)..(2): PHOSPHORYLATION; tyrosine at position 2 is phosphorylated
AYHEQLSVAEITNACFEPANQMVK

SEQ ID NO: 164 - protein, 12 aa, Homo sapiens
  MOD_RES (9)..(9): PHOSPHORYLATION; tyrosine at position 3 is phosphorylated
LDHKFDLMYAKR

SEQ ID NO: 165 - protein, 29 aa, Homo sapiens
  MOD_RES (29)..(29): PHOSPHORYLATION; tyrosine at position 29 is phosphorylated
EDMAALEKDYEEVGVDSVEGEGEEEGEEY

SEQ ID NO: 166 - protein, 19 aa, Homo sapiens
  MOD_RES (3)..(3): PHOSPHORYLATION; tyrosine at position 3 is phosphorylated
GHYTEGAELVDSVLDVVRK

SEQ ID NO: 167 - protein, 25 aa, Homo sapiens
  MOD_RES (6)..(6): PHOSPHORYLATION; tyrosine at position 6 is phosphorylated
LTTPTYGDLNHLVSATMSGVTTCLR

SEQ ID NO: 168 - protein, 12 aa, Homo sapiens
  MOD_RES (5)..(5): PHOSPHORYLATION; tyrosine at position 5 is phosphorylated
ISVYYNEATGGK

SEQ ID NO: 169 - protein, 12 aa, Homo sapiens
  MOD_RES (6)..(6): PHOSPHORYLATION; tyrosine at position 6 is phosphorylated
DVRQQYESVAAK

SEQ ID NO: 170 - protein, 32 aa, Homo sapiens
  MOD_RES (17)..(17): PHOSPHORYLATION; tyrosine at position 17 is phosphorylated
YALLIVDSATALYRTDYSGRGELSARQMHLAR

SEQ ID NO: 171 - protein, 21 aa, Homo sapiens
  MOD_RES (17)..(17): PHOSPHORYLATION; tyrosine at position 17 is phosphorylated
RKLIRYVTAFNGELEDYMSDR

SEQ ID NO: 172 - protein, 22 aa, Homo sapiens
  MOD_RES (7)..(7): PHOSPHORYLATION; tyrosine at position 7 is phosphorylated
TFQMAEYLKLEFIKEIGYTHMK

SEQ ID NO: 173 - protein, 18 aa, Homo sapiens
  MOD_RES (9)..(9): PHOSPHORYLATION; tyrosine at position 9is phosphorylated
MATDEENVYGLEENAQSR

SEQ ID NO: 174 - protein, 15 aa, Homo sapiens
  MOD_RES (5)..(5): PHOSPHORYLATION; tyrosine at position 5 is phosphorylated
HSVNYKPPAQKSIQE

SEQ ID NO: 175 - protein, 19 aa, Homo sapiens
  MOD_RES (18)..(18): PHOSPHORYLATION; tyrosine at position 18 is phosphorylated
SLKELQEMDKDDESLIKYK

SEQ ID NO: 176 - protein, 19 aa, Homo sapiens
  MOD_RES (10)..(10): PHOSPHORYLATION; tyrosine at position 10 is phosphorylated
RPVSFPETPYTVSPAGADR

SEQ ID NO: 177 - protein, 13 aa, Homo sapiens
  MOD_RES (7)..(7): PHOSPHORYLATION; tyrosine at position 7 is phosphorylated
IIHEDGYSEEECR

SEQ ID NO: 178 - protein, 31 aa, Homo sapiens
  MOD_RES (23)..(23): PHOSPHORYLATION; tyrosine at position 23 is phosphorylated
QMRIIHGAGYSEEERKGFRPLVYQNIFVSMR

SEQ ID NO: 179 - protein, 9 aa, Homo sapiens
  MOD_RES (9)..(9): PHOSPHORYLATION; tyrosine at position 9 is phosphorylated
NNLKECGLY

SEQ ID NO: 180 - protein, 25 aa, Homo sapiens
  MOD_RES (22)..(22): PHOSPHORYLATION; tyrosine at position 22 is phosphorylated
GNTPPPQKKEDDFLNNAMSSLYSGW

SEQ ID NO: 181 - protein, 15 aa, Homo sapiens
  MOD_RES (8)..(8): PHOSPHORYLATION; tyrosine at position 8 is phosphorylated
WDDSSDSYWKKETSK

SEQ ID NO: 182 - protein, 19 aa, Homo sapiens
  MOD_RES (14)..(14): PHOSPHORYLATION; tyrosine at position 14 is phosphorylated
EAEAAQGQAGDETYLDIFR

SEQ ID NO: 183 - protein, 22 aa, Homo sapiens
  MOD_RES (18)..(18): PHOSPHORYLATION; tyrosine at position 18 is phosphorylated
LTPLLSGWLDKLSPQGNYVFQR

SEQ ID NO: 184 - protein, 14 aa, Homo sapiens
  MOD_RES (4)..(4): PHOSPHORYLATION; tyrosine at position 4 is phosphorylated
SLMYFGSDKDPFPK

SEQ ID NO: 185 - protein, 23 aa, Homo sapiens
  MOD_RES (18)..(18): PHOSPHORYLATION; tyrosine at position 18 is phosphorylated
QSWAAALQEAVTETLSDYEVAEK

SEQ ID NO: 186 - protein, 15 aa, Homo sapiens
  MOD_RES (8)..(8): PHOSPHORYLATION; tyrosine at position 8 is phosphorylated
ATYSGFLYCSPVSNK

SEQ ID NO: 187 - protein, 9 aa, Homo sapiens
  MOD_RES (3)..(3): PHOSPHORYLATION; tyrosine at position 3 is phosphorylated
TLYLQGEGR

SEQ ID NO: 188 - protein, 15 aa, Homo sapiens
  MOD_RES (8)..(8): PHOSPHORYLATION; tyrosine at position 3 is phosphorylated
TGSVGGTYRQKSMPE

SEQ ID NO: 189 - protein, 17 aa, Homo sapiens
  MOD_RES (13)..(13): PHOSPHORYLATION; tyrosine at position 13 is phosphorylated
GDKEEQEVSWTQYTQRK

SEQ ID NO: 190 - protein, 22 aa, Homo sapiens
  MOD_RES (3)..(3): PHOSPHORYLATION; tyrosine at position 3 is phosphorylated
EIYMTFLSSKASSQVNVEGQSR

SEQ ID NO: 191 - protein, 19 aa, Homo sapiens
  MOD_RES (3)..(3): PHOSPHORYLATION; tyrosine at position 3 is phosphorylated
NIYQEFLSSQALSPVNIDR

SEQ ID NO: 192 - protein, 15 aa, Homo sapiens
  MOD_RES (7)..(7): PHOSPHORYLATION; tyrosine at position 7 is phosphorylated
NVETVTYKNLKFQVW

SEQ ID NO: 193 - protein, 26 aa, Homo sapiens
  MOD_RES (7)..(7): PHOSPHORYLATION; tyrosine at position 7 is phosphorylated
ARLSSAYLLGVPGSEQPDRAGSLELR

SEQ ID NO: 194 - protein, 15 aa, Homo sapiens
  MOD_RES (8)..(8): PHOSPHORYLATION; tyrosine at position 8 is phosphorylated
KVVENEIYSESHRVH

SEQ ID NO: 195 - protein, 11 aa, Homo sapiens
  MOD_RES (7)..(7): PHOSPHORYLATION; tyrosine at position 7 is phosphorylated
KPSEEEYVIRK

SEQ ID NO: 196 - protein, 23 aa, Homo sapiens
  MOD_RES (9)..(9): PHOSPHORYLATION; tyrosine at position 9is phosphorylated
EKKLAPITYPQGLALAKEIDSVK

SEQ ID NO: 197 - protein, 19 aa, Homo sapiens
  MOD_RES (9)..(9): PHOSPHORYLATION; tyrosine at position 9 is phosphorylated
SLPAGDALYLSFNPPQPSR

SEQ ID NO: 198 - protein, 15 aa, Homo sapiens
  MOD_RES (8)..(8): PHOSPHORYLATION; tyrosine at position 8 is phosphorylated
GWWRGEIYGRVGWFP

SEQ ID NO: 199 - protein, 27 aa, Homo sapiens
  MOD_RES (9)..(9): PHOSPHORYLATION; tyrosine at position 9 is phosphorylated
RLSGGSHSYGGESPRLSPCSSIDKLSK

SEQ ID NO: 200 - protein, 22 aa, Homo sapiens
  MOD_RES (1)..(1): PHOSPHORYLATION; tyrosine at position 1 is phosphorylated
YAGDIIKALELSGTAVPPDLEK

SEQ ID NO: 201 - protein, 17 aa, Homo sapiens
  MOD_RES (16)..(16): PHOSPHORYLATION; tyrosine at position 16 is phosphorylated
QEVPSWLENMAYEHHYK

SEQ ID NO: 202 - protein, 15 aa, Homo sapiens
  MOD_RES (11)..(11): PHOSPHORYLATION; tyrosine at position 11 is phosphorylated
LQTEAQEDDWYDCHR

SEQ ID NO: 203 - protein, 12 aa, Homo sapiens
  MOD_RES (3)..(3): PHOSPHORYLATION; tyrosine at position 3 is phosphorylated
GSYVSIHSSGFR

SEQ ID NO: 204 - protein, 15 aa, Homo sapiens
  MOD_RES (2)..(2): PHOSPHORYLATION; tyrosine at position 2 is phosphorylated
LYAATDAYAGFIIYR

SEQ ID NO: 205 - protein, 11 aa, Homo sapiens
  MOD_RES (5)..(5): PHOSPHORYLATION; tyrosine at position 5 is phosphorylated
CNTLSSPNQYQ

SEQ ID NO: 206 - protein, 16 aa, Homo sapiens
  MOD_RES (10)..(10): PHOSPHORYLATION; tyrosine at position 10 is phosphorylated
TSQISTMQQYNLCVYR

SEQ ID NO: 207 - protein, 13 aa, Homo sapiens
  MOD_RES (11)..(11): PHOSPHORYLATION; tyrosine at position 11 is phosphorylated
LLPQLTYLDGYDR

SEQ ID NO: 208 - protein, 18 aa, Homo sapiens
  MOD_RES (17)..(17): PHOSPHORYLATION; tyrosine at position 17 is phosphorylated
TSDQIHFFFAKLNCRLYR

SEQ ID NO: 209 - protein, 19 aa, Homo sapiens
  MOD_RES (16)..(16): PHOSPHORYLATION; tyrosine at position 16 is phosphorylated
IRLQAYHTQTTPLIEYYRK

SEQ ID NO: 210 - protein, 29 aa, Homo sapiens
  MOD_RES (5)..(5): PHOSPHORYLATION; tyrosine at position 5 is phosphorylated
RIQTYLQSTKPIIDLYEEMGKVKKIDASK

SEQ ID NO: 211 - protein, 29 aa, Homo sapiens
  MOD_RES (16)..(16): PHOSPHORYLATION; tyrosine at position 16 is phosphorylated
RIQTYLQSTKPIIDLYEEMGKVKKIDASK

SEQ ID NO: 212 - protein, 12 aa, Homo sapiens
  MOD_RES (6)..(6): PHOSPHORYLATION; tyrosine at position 6 is phosphorylated
MISGMYLGEIVR

SEQ ID NO: 213 - protein, 16 aa, Homo sapiens
  MOD_RES (12)..(12): PHOSPHORYLATION; tyrosine at position 12 is phosphorylated
QGFFDIPVDNLYAEPA

SEQ ID NO: 214 - protein, 28 aa, Homo sapiens
  MOD_RES (1)..(1): PHOSPHORYLATION; tyrosine at position 1 is phosphorylated
YPPNQNIPRQAIKPNPLQMAFLAQQAIK

SEQ ID NO: 215 - protein, 20 aa, Homo sapiens
  MOD_RES (12)..(12): PHOSPHORYLATION; tyrosine at position 12 is phosphorylated
DCQLNAHKDHQYQFLEDAVR

SEQ ID NO: 216 - protein, 8 aa, Homo sapiens
  MOD_RES (3)..(3): PHOSPHORYLATION; tyrosine at position 3 is phosphorylated
LIYFQLHR

SEQ ID NO: 217 - protein, 20 aa, Homo sapiens
  MOD_RES (5)..(5): PHOSPHORYLATION; tyrosine at position 5 is phosphorylated
HSQHYQIPDDFVADVRLIFK

SEQ ID NO: 218 - protein, 22 aa, Homo sapiens
  MOD_RES (5)..(5): PHOSPHORYLATION; tyrosine at position 5 is phosphorylated
QKMLYAATRATLKKEFGGGHIK

SEQ ID NO: 219 - protein, 21 aa, Homo sapiens
  MOD_RES (19)..(19): PHOSPHORYLATION; tyrosine at position 19 is phosphorylated
YEIDSLIGKGSFGQVVKAYDR

SEQ ID NO: 220 - protein, 19 aa, Homo sapiens
  MOD_RES (5)..(5): PHOSPHORYLATION; tyrosine at position 5 is phosphorylated
SGPFYLEGLDLSPKEIVQK

SEQ ID NO: 221 - protein, 24 aa, Homo sapiens
  MOD_RES (9)..(9): PHOSPHORYLATION; tyrosine at position 9 is phosphorylated
GTTEEMKYVLGQLVGLNSPNSILK

SEQ ID NO: 222 - protein, 19 aa, Homo sapiens
  MOD_RES (8)..(8): PHOSPHORYLATION; tyrosine at position 8 is phosphorylated
MTDVETTYADFIASGRTGR

SEQ ID NO: 223 - protein, 11 aa, Homo sapiens
  MOD_RES (6)..(6): PHOSPHORYLATION; tyrosine at position 6 is phosphorylated
SPNEEYVEVGR

SEQ ID NO: 224 - protein, 10 aa, Homo sapiens
  MOD_RES (3)..(3): PHOSPHORYLATION; tyrosine at position 3 is phosphorylated
MIYLQTLLAK

SEQ ID NO: 225 - protein, 18 aa, Homo sapiens
  MOD_RES (17)..(17): PHOSPHORYLATION; tyrosine at position 17 is phosphorylated
SQHGAGSSVGDASRPPYR

SEQ ID NO: 226 - protein, 18 aa, Homo sapiens
  MOD_RES (5)..(5): PHOSPHORYLATION; tyrosine at position 5 is phosphorylated
VPELYEIHKEFYDGLFPR

SEQ ID NO: 227 - protein, 18 aa, Homo sapiens
  MOD_RES (12)..(12): PHOSPHORYLATION; tyrosine at position 12 is phosphorylated
VPELYEIHKEFYDGLFPR

SEQ ID NO: 228 - protein, 12 aa, Homo sapiens
  MOD_RES (10)..(10): PHOSPHORYLATION; tyrosine at position 10 is phosphorylated
SYTFLISSDYER

SEQ ID NO: 229 - protein, 34 aa, Homo sapiens
  MOD_RES (12)..(12): PHOSPHORYLATION; tyrosine at position 12 is phosphorylated
EVEGTSYHESLYNALQSLRDREFSTFYESLKYAR

SEQ ID NO: 230 - protein, 27 aa, Homo sapiens
  MOD_RES (12)..(12): PHOSPHORYLATION; tyrosine at position 12 is phosphorylated
VQGGVPAGSDEYEDECPHLIALSSLNR

SEQ ID NO: 231 - protein, 18 aa, Homo sapiens
  MOD_RES (17)..(17): PHOSPHORYLATION; tyrosine at position 17 is phosphorylated
EFRPFRDEENVGAMNQYR

SEQ ID NO: 232 - protein, 32 aa, Homo sapiens
  MOD_RES (17)..(17): PHOSPHORYLATION; tyrosine at position 17 is phosphorylated
GVDVSLQGKDAWLPLHYAAWQGHLPIVKLLAK

SEQ ID NO: 233 - protein, 26 aa, Homo sapiens
  MOD_RES (12)..(12): PHOSPHORYLATION; tyrosine at position 12 is phosphorylated
LSKFDEQRTATYITELANALSYCHSK

SEQ ID NO: 234 - protein, 26 aa, Homo sapiens
  MOD_RES (22)..(22): PHOSPHORYLATION; tyrosine at position 22 is phosphorylated
LSKFDEQRTATYITELANALSYCHSK

SEQ ID NO: 235 - protein, 16 aa, Homo sapiens
  MOD_RES (8)..(8): PHOSPHORYLATION; tyrosine at position 8 is phosphorylated
TLNHQYDYTFDWTMLK

SEQ ID NO: 236 - protein, 10 aa, Homo sapiens
  MOD_RES (6)..(6): PHOSPHORYLATION; tyrosine at position 6 is phosphorylated
EPPKAYREDK

SEQ ID NO: 237 - protein, 19 aa, Homo sapiens
  MOD_RES (12)..(12): PHOSPHORYLATION; tyrosine at position 12 is phosphorylated
YENLGLVGEGSYGMVMKCR

SEQ ID NO: 238 - protein, 12 aa, Homo sapiens
  MOD_RES (9)..(9): PHOSPHORYLATION; tyrosine at position 9 is phosphorylated
FVVSLAYAYETK

SEQ ID NO: 239 - protein, 21 aa, Homo sapiens
  MOD_RES (4)..(4): PHOSPHORYLATION; tyrosine at position 4 is phosphorylated
DNLYFVMDYIPGGDMMSLLIR

SEQ ID NO: 240 - protein, 18 aa, Homo sapiens
  MOD_RES (15)..(15): PHOSPHORYLATION; tyrosine at position 15 is phosphorylated
FSDQAGPAIPTSNSYSKK

SEQ ID NO: 241 - protein, 21 aa, Homo sapiens
  MOD_RES (7)..(7): PHOSPHORYLATION; tyrosine at position 7 is phosphorylated
DQQNLPYGVTPASPSGHSQGR

SEQ ID NO: 242 - protein, 18 aa, Homo sapiens
  MOD_RES (16)..(16): PHOSPHORYLATION; tyrosine at position 16 is phosphorylated
RYSDHAGPAIPSVVAYPK

SEQ ID NO: 243 - protein, 15 aa, Homo sapiens
  MOD_RES (8)..(8): PHOSPHORYLATION
SAVKNEEYFMFPEPK

SEQ ID NO: 244 - protein, 18 aa, Homo sapiens
  MOD_RES (12)..(12): PHOSPHORYLATION; tyrosine at position 12 is phosphorylated
ASQSAGDINTIYQPPEPR

SEQ ID NO: 245 - protein, 11 aa, Homo sapiens
  MOD_RES (8)..(8): PHOSPHORYLATION; tyrosine at position 8 is phosphorylated
SNSAWQIYLQR

SEQ ID NO: 246 - protein, 16 aa, Homo sapiens
  MOD_RES (13)..(13): PHOSPHORYLATION; tyrosine at position 13 is phosphorylated
GQSPPSKDGSGDYQSR

SEQ ID NO: 247 - protein, 20 aa, Homo sapiens
  MOD_RES (8)..(8): PHOSPHORYLATION; tyrosine at position 8 is phosphorylated
QEGSIEVYEDAGSHYLCLLK

SEQ ID NO: 248 - protein, 20 aa, Homo sapiens
  MOD_RES (15)..(15): PHOSPHORYLATION; tyrosine at position 15 is phosphorylated
QEGSIEVYEDAGSHYLCLLK

SEQ ID NO: 249 - protein, 26 aa, Homo sapiens
  MOD_RES (1)..(1): PHOSPHORYLATION; tyrosine at position 1 is phosphorylated
YQNLSPVGSGAYGSVCAAFDTKTGLR

SEQ ID NO: 250 - protein, 24 aa, Homo sapiens
  MOD_RES (6)..(6): PHOSPHORYLATION; tyrosine at position 6 is phosphorylated
LGEGSYATVYKGISRINGQLVALK

SEQ ID NO: 251 - protein, 16 aa, Homo sapiens
  MOD_RES (2)..(2): PHOSPHORYLATION; tyrosine at position 2 is phosphorylated
FYAAEICIALNFLHER

SEQ ID NO: 252 - protein, 29 aa, Homo sapiens
  MOD_RES (1)..(1): PHOSPHORYLATION; tyrosine at position 1 is phosphorylated
YSDHAGPGIPSVVAYPKRSQTSTADSDLK

SEQ ID NO: 253 - protein, 9 aa, Homo sapiens
  MOD_RES (4)..(4): PHOSPHORYLATION; tyrosine at position 4 is phosphorylated
YTLYKKEEE

SEQ ID NO: 254 - protein, 13 aa, Homo sapiens
  MOD_RES (8)..(8): PHOSPHORYLATION; tyrosine at position 8 is phosphorylated
QEDAPVVYLGSFR

SEQ ID NO: 255 - protein, 22 aa, Homo sapiens
  MOD_RES (20)..(20): PHOSPHORYLATION; tyrosine at position 20 is phosphorylated
QIQSLSVHDSQGPPNANEDYRE

SEQ ID NO: 256 - protein, 18 aa, Homo sapiens
  MOD_RES (3)..(3): PHOSPHORYLATION; tyrosine at position 3 is phosphorylated
RGYLLGINLGEGSYAKVK

SEQ ID NO: 257 - protein, 28 aa, Homo sapiens
  MOD_RES (8)..(8): PHOSPHORYLATION; tyrosine at position 8 is phosphorylated
SKRFRVRYDGIHYLDIVDCKSYDTGEVK

SEQ ID NO: 258 - protein, 28 aa, Homo sapiens
  MOD_RES (13)..(13): PHOSPHORYLATION; tyrosine at position 13 is phosphorylated
SKRFRVRYDGIHYLDIVDCKSYDTGEVK

SEQ ID NO: 259 - protein, 28 aa, Homo sapiens
  MOD_RES (22)..(22): PHOSPHORYLATION; tyrosine at position 22 is phosphorylated
SKRFRVRYDGIHYLDIVDCKSYDTGEVK

SEQ ID NO: 260 - protein, 11 aa, Homo sapiens
  MOD_RES (8)..(8): PHOSPHORYLATION; tyrosine at position 8 is phosphorylated
DLIPNGEYFFR

SEQ ID NO: 261 - protein, 29 aa, Homo sapiens
  MOD_RES (1)..(1): PHOSPHORYLATION; tyrosine at position 1 is phosphorylated
YEQLPEDVGHCFVTWADKFQMYVTYCKNK

SEQ ID NO: 262 - protein, 29 aa, Homo sapiens
  MOD_RES (22)..(22): PHOSPHORYLATION; tyrosine at position 22 is phosphorylated
YEQLPEDVGHCFVTWADKFQMYVTYCKNK

SEQ ID NO: 263 - protein, 29 aa, Homo sapiens
  MOD_RES (25)..(25): PHOSPHORYLATION; tyrosine at position 25 is phosphorylated
YEQLPEDVGHCFVTWADKFQMYVTYCKNK

SEQ ID NO: 264 - protein, 23 aa, Homo sapiens
  MOD_RES (4)..(4): PHOSPHORYLATION; tyrosine at position 4 is phosphorylated
TRFYGAEIVSALDYLHSGKIVYR

SEQ ID NO: 265 - protein, 23 aa, Homo sapiens
  MOD_RES (22)..(22): PHOSPHORYLATION; tyrosine at position 22 is phosphorylated
TRFYGAEIVSALDYLHSGKIVYR

SEQ ID NO: 266 - protein, 12 aa, Homo sapiens
  MOD_RES (5)..(5): PHOSPHORYLATION; tyrosine at position 5 is phosphorylated
GHMNYEGPGMAR

SEQ ID NO: 267 - protein, 22 aa, Homo sapiens
  MOD_RES (21)..(21): PHOSPHORYLATION
IADPEHDHTGFLTEYVATRWYR

SEQ ID NO: 268 - protein, 32 aa, Homo sapiens
  MOD_RES (30)..(30): PHOSPHORYLATION; tyrosine at position 30 is phosphorylated
RLHCYASWANSSGTIELVKKGCWLDDFNCYDR

SEQ ID NO: 269 - protein, 14 aa, Homo sapiens
  MOD_RES (2)..(2): PHOSPHORYLATION; tyrosine at position 2 is phosphorylated
RYMAPEVLDESLNK

SEQ ID NO: 270 - protein, 12 aa, Homo sapiens
  MOD_RES (7)..(7): PHOSPHORYLATION; tyrosine at position 7 is phosphorylated
CLDEGGYYISPR

SEQ ID NO: 271 - protein, 17 aa, Homo sapiens
  MOD_RES (12)..(12): PHOSPHORYLATION; tyrosine at position 12 is phosphorylated
HYVVCSTPQSQYYLAEK

SEQ ID NO: 272 - protein, 16 aa, Homo sapiens
  MOD_RES (12)..(12): PHOSPHORYLATION; tyrosine at position 12 is phosphorylated
MVRPDNCPEELYQLMR

SEQ ID NO: 273 - protein, 12 aa, Homo sapiens
  MOD_RES (4)..(4): PHOSPHORYLATION; tyrosine at position 4 is phosphorylated
IKSYSFPKPGHR

SEQ ID NO: 274 - protein, 10 aa, Homo sapiens
  MOD_RES (5)..(5): PHOSPHORYLATION; tyrosine at position 5 is phosphorylated
LRNYYYDVVN

SEQ ID NO: 275 - protein, 10 aa, Homo sapiens
  MOD_RES (6)..(6): PHOSPHORYLATION; tyrosine at position 6 is phosphorylated
LRNYYYDVVN

SEQ ID NO: 276 - protein, 15 aa, Homo sapiens
  MOD_RES (8)..(8): PHOSPHORYLATION; tyrosine at position 8 is phosphorylated
RYFLDDQYTSSSGAK

SEQ ID NO: 277 - protein, 17 aa, Homo sapiens
  MOD_RES (8)..(8): PHOSPHORYLATION; tyrosine at position 8 is phosphorylated
LTADSSHYLCHEVAPPR

SEQ ID NO: 278 - protein, 20 aa, Homo sapiens
  MOD_RES (3)..(3): PHOSPHORYLATION; tyrosine at position 3 is phosphorylated
VSLYCYDPTNDGTGEMVAVK

SEQ ID NO: 279 - protein, 10 aa, Homo sapiens
  MOD_RES (4)..(4): PHOSPHORYLATION; tyrosine at position 4 is phosphorylated
HSWYHGPVSR

SEQ ID NO: 280 - protein, 14 aa, Homo sapiens
  MOD_RES (7)..(7): PHOSPHORYLATION; tyrosine at position 7 is phosphorylated
EEADGVYAASGGLR

SEQ ID NO: 281 - protein, 19 aa, Homo sapiens
  MOD_RES (14)..(14): PHOSPHORYLATION; tyrosine at position 14 is phosphorylated
LGAGQFGEVWMGYYNNSTK

SEQ ID NO: 282 - protein, 12 aa, Homo sapiens
  MOD_RES (6)..(6): PHOSPHORYLATION; tyrosine at position 6 is phosphorylated
QLNGTYAIAGGK

SEQ ID NO: 283 - protein, 10 aa, Homo sapiens
  MOD_RES (4)..(4): PHOSPHORYLATION; tyrosine at position 4 is phosphorylated
MPWYHSSLTR

SEQ ID NO: 284 - protein, 20 aa, Homo sapiens
  MOD_RES (15)..(15): PHOSPHORYLATION
STSASINNLKPGTVYVFQIR

SEQ ID NO: 285 - protein, 11 aa, Homo sapiens
  MOD_RES (7)..(7): PHOSPHORYLATION; tyrosine at position 7 is phosphorylated
VMNATAYGISK

SEQ ID NO: 286 - protein, 29 aa, Homo sapiens
  MOD_RES (13)..(13): PHOSPHORYLATION; tyrosine at position 13 is phosphorylated
TWTEIFKEHNFSFYPTFQSHPNSSMPGSR

SEQ ID NO: 287 - protein, 31 aa, Homo sapiens
  MOD_RES (27)..(27): PHOSPHORYLATION; tyrosine at position 27 is phosphorylated
EVQIHPDSDQISGLHGNSFHSEDEIEYENQK

SEQ ID NO: 288 - protein, 16 aa, Homo sapiens
  MOD_RES (15)..(15): PHOSPHORYLATION; tyrosine at position 15 is phosphorylated
KAANMSDVSDLRIFYR

SEQ ID NO: 289 - protein, 11 aa, Homo sapiens
  MOD_RES (11)..(11): PHOSPHORYLATION; tyrosine at position 11 is phosphorylated
NLLGACEHRGY

SEQ ID NO: 290 - protein, 9 aa, Homo sapiens
  MOD_RES (3)..(3): PHOSPHORYLATION; tyrosine at position 3 is phosphorylated
KIYSGDYYR

SEQ ID NO: 291 - protein, 18 aa, Homo sapiens
  MOD_RES (7)..(7): PHOSPHORYLATION; tyrosine at position 7 is phosphorylated
RPHGDIYGINQALGATAK

SEQ ID NO: 292 - protein, 7 aa, Homo sapiens
  MOD_RES (1)..(1): PHOSPHORYLATION; tyrosine at position 1 is phosphorylated
YISLSEK

SEQ ID NO: 293 - protein, 19 aa, Homo sapiens
  MOD_RES (9)..(9): PHOSPHORYLATION; tyrosine at position 9 is phosphorylated
ERVPFILTYDFIHVIQQGK

SEQ ID NO: 294 - protein, 31 aa, Homo sapiens
  MOD_RES (29)..(29): PHOSPHORYLATION; tyrosine at position 29 is phosphorylated
RHGNLFITLFALMLTAGLPELTSVKDIQYLK

SEQ ID NO: 295 - protein, 15 aa, Homo sapiens
  MOD_RES (8)..(8): PHOSPHORYLATION; tyrosine at position 8 is phosphorylated
EVAPHPVYYPALEKI

SEQ ID NO: 296 - protein, 24 aa, Homo sapiens
  MOD_RES (18)..(18): PHOSPHORYLATION; tyrosine at position 18 is phosphorylated
QNADPSLISWDEPGVDFYSKPAGR

SEQ ID NO: 297 - protein, 11 aa, Homo sapiens
  MOD_RES (5)..(5): PHOSPHORYLATION; tyrosine at position 5 is phosphorylated
QAAEYREIDKR

SEQ ID NO: 298 - protein, 18 aa, Homo sapiens
  MOD_RES (15)..(15): PHOSPHORYLATION; tyrosine at position 15 is phosphorylated
GFFICDQPYEPVSPYSCK

SEQ ID NO: 299 - protein, 11 aa, Homo sapiens
  MOD_RES (3)..(3): PHOSPHORYLATION; tyrosine at position 3 is phosphorylated
EVYELLDSPGK

SEQ ID NO: 300 - protein, 24 aa, Homo sapiens
  MOD_RES (16)..(16): PHOSPHORYLATION; tyrosine at position 16 is phosphorylated
GYQRRWFVLSNGLLSYYRSKAEMR

SEQ ID NO: 301 - protein, 24 aa, Homo sapiens
  MOD_RES (17)..(17): PHOSPHORYLATION; tyrosine at position 17 is phosphorylated
GYQRRWFVLSNGLLSYYRSKAEMR

SEQ ID NO: 302 - protein, 24 aa, Homo sapiens
  MOD_RES (17)..(17): PHOSPHORYLATION; tyrosine at position 17 is phosphorylated
EAEAMKATEDGTPYDPYKALWFER

SEQ ID NO: 303 - protein, 21 aa, Homo sapiens
  MOD_RES (14)..(14): PHOSPHORYLATION; tyrosine at position 14 is phosphorylated
LSIFDANESGFESYEALPQHK

SEQ ID NO: 304 - protein, 15 aa, Homo sapiens
  MOD_RES (9)..(9): PHOSPHORYLATION; tyrosine at position 9 is phosphorylated
KEPVDEDLYPEHYRK

SEQ ID NO: 305 - protein, 29 aa, Homo sapiens
  MOD_RES (3)..(3): PHOSPHORYLATION; tyrosine at position 3 is phosphorylated
EAYHLPMPMAAEPLPSSSVSGEEARLPPR

SEQ ID NO: 306 - protein, 20 aa, Homo sapiens
  MOD_RES (5)..(5): PHOSPHORYLATION; tyrosine at position 5 is phosphorylated
TTPEYVFHCQSAVMSATFAK

SEQ ID NO: 307 - protein, 13 aa, Homo sapiens
  MOD_RES (8)..(8): PHOSPHORYLATION; tyrosine at position 8 is phosphorylated
DGTSFGEYGGWYK

SEQ ID NO: 308 - protein, 15 aa, Homo sapiens
  MOD_RES (13)..(13): PHOSPHORYLATION; tyrosine at position 13 is phosphorylated
TPTCQSSTDCSPYAR

SEQ ID NO: 309 - protein, 13 aa, Homo sapiens
  MOD_RES (8)..(8): PHOSPHORYLATION; tyrosine at position 8 is phosphorylated
DSAPYGEYGSWYK

SEQ ID NO: 310 - protein, 20 aa, Homo sapiens
  MOD_RES (17)..(17): PHOSPHORYLATION; tyrosine at position 17 is phosphorylated
NTTQNTGYSSGTQNANYPVR

SEQ ID NO: 311 - protein, 11 aa, Homo sapiens
  MOD_RES (7)..(7): PHOSPHORYLATION; tyrosine at position 7 is phosphorylated
IQSHCSYTYGR

SEQ ID NO: 312 - protein, 22 aa, Homo sapiens
  MOD_RES (17)..(17): PHOSPHORYLATION; tyrosine at position 17 is phosphorylated
KIGEMPPHIFAIADNCYFNMKR

SEQ ID NO: 313 - protein, 23 aa, Homo sapiens
  MOD_RES (4)..(4): PHOSPHORYLATION; tyrosine at position 4 is phosphorylated
FVKYLRNFIHGGPPGYAPYCEER

SEQ ID NO: 314 - protein, 15 aa, Homo sapiens
  MOD_RES (4)..(4): PHOSPHORYLATION; tyrosine at position 4 is phosphorylated
AVIYNPATQADWTAK

SEQ ID NO: 315 - protein, 13 aa, Homo sapiens
  MOD_RES (4)..(4): PHOSPHORYLATION; tyrosine at position 4 is phosphorylated
VIQYLAYVASSHK

SEQ ID NO: 316 - protein, 14 aa, Homo sapiens
  MOD_RES (8)..(8): PHOSPHORYLATION; tyrosine at position 8 is phosphorylated
KVIQYLAYVASSHK

SEQ ID NO: 317 - protein, 15 aa, Homo sapiens
  MOD_RES (8)..(8): PHOSPHORYLATION; tyrosine at position 8 is phosphorylated
ARGPQSNYGGPYPAA

SEQ ID NO: 318 - protein, 15 aa, Homo sapiens
  MOD_RES (8)..(8): PHOSPHORYLATION; tyrosine at position 8 is phosphorylated
QSNYGGPYPAAPTFG

SEQ ID NO: 319 - protein, 30 aa, Homo sapiens
  MOD_RES (1)..(1): PHOSPHORYLATION; tyrosine at position 1 is phosphorylated
YEINVLRNRINDNQKVSKTRGKAKVTGRWK

SEQ ID NO: 320 - protein, 22 aa, Homo sapiens
  MOD_RES (6)..(6): PHOSPHORYLATION; tyrosine at position 6 is phosphorylated
ALEKDYEEVGADSADGEDEGEE

SEQ ID NO: 321 - protein, 10 aa, Homo sapiens
  MOD_RES (5)..(5): PHOSPHORYLATION; tyrosine at position 5 is phosphorylated
VTIEYYSQLK

SEQ ID NO: 322 - protein, 21 aa, Homo sapiens
  MOD_RES (2)..(2): PHOSPHORYLATION; tyrosine at position 2 is phosphorylated
FYAYNPLAGGLLTGKYKYEDK

SEQ ID NO: 323 - protein, 21 aa, Homo sapiens
  MOD_RES (4)..(4): PHOSPHORYLATION; tyrosine at position 4 is phosphorylated
FYAYNPLAGGLLTGKYKYEDK

SEQ ID NO: 324 - protein, 13 aa, Homo sapiens
  MOD_RES (11)..(11): PHOSPHORYLATION; tyrosine at position 11 is phosphorylated
LGCQDAFPEVYDK

SEQ ID NO: 325 - protein, 31 aa, Homo sapiens
  MOD_RES (30)..(30): PHOSPHORYLATION; tyrosine at position 30 is phosphorylated
LQGPETARELYSILQHKGLVDKFPLFMAVYK

SEQ ID NO: 326 - protein, 25 aa, Homo sapiens
  MOD_RES (21)..(21): PHOSPHORYLATION; tyrosine at position 21 is phosphorylated
GRLVNVSSMGGGAPMERLASYGSSK

SEQ ID NO: 327 - protein, 16 aa, Homo sapiens
  MOD_RES (2)..(2): PHOSPHORYLATION; tyrosine at position 2 is phosphorylated
AYDGRFKDIFQEIFDK

SEQ ID NO: 328 - protein, 23 aa, Homo sapiens
  MOD_RES (1)..(1): PHOSPHORYLATION; tyrosine at position 1 is phosphorylated
YVVSMGSCANGGGYYHYSYSVVR

SEQ ID NO: 329 - protein, 23 aa, Homo sapiens
  MOD_RES (17)..(17): PHOSPHORYLATION; tyrosine at position 17 is phosphorylated
YVVSMGSCANGGGYYHYSYSVVR

SEQ ID NO: 330 - protein, 11 aa, Homo sapiens
  MOD_RES (2)..(2): PHOSPHORYLATION; tyrosine at position 2 is phosphorylated
EYVSGFGKAMR

SEQ ID NO: 331 - protein, 27 aa, Homo sapiens
  MOD_RES (4)..(4): PHOSPHORYLATION; tyrosine at position 4 is phosphorylated
LSFYSGHSSFSMYCMLFVALYLQARMK

SEQ ID NO: 332 - protein, 27 aa, Homo sapiens
  MOD_RES (21)..(21): PHOSPHORYLATION; tyrosine at position 21 is phosphorylated
LSFYSGHSSFSMYCMLFVALYLQARMK

SEQ ID NO: 333 - protein, 36 aa, Homo sapiens
  MOD_RES (5)..(5): PHOSPHORYLATION; tyrosine at position 5 is phosphorylated
WGLSYMGTNMHSLQLTISRRNGSETHTGNTGSEAAF

SEQ ID NO: 334 - protein, 20 aa, Homo sapiens
  MOD_RES (10)..(10): PHOSPHORYLATION; tyrosine at position 10 is phosphorylated
SKDGSEDKFYSHKKILQLIK

SEQ ID NO: 335 - protein, 15 aa, Homo sapiens
  MOD_RES (4)..(4): PHOSPHORYLATION; tyrosine at position 4 is phosphorylated
GRDYRDNTELPHHGK

SEQ ID NO: 336 - protein, 16 aa, Homo sapiens
  MOD_RES (5)..(5): PHOSPHORYLATION; tyrosine at position 5 is phosphorylated
SMDGYESYGECVVALK

SEQ ID NO: 337 - protein, 15 aa, Homo sapiens
  MOD_RES (8)..(8): PHOSPHORYLATION; tyrosine at position 8 is phosphorylated
KTLSEVDYAPAGPAR

SEQ ID NO: 338 - protein, 15 aa, Homo sapiens
  MOD_RES (8)..(8): PHOSPHORYLATION; tyrosine at position 8 is phosphorylated
PRGLPSDYGRPLSFP

SEQ ID NO: 339 - protein, 20 aa, Homo sapiens
  MOD_RES (6)..(6): PHOSPHORYLATION; tyrosine at position 6 is phosphorylated
ALDMSYDHKPEDEVELARIK

SEQ ID NO: 340 - protein, 12 aa, Homo sapiens
  MOD_RES (10)..(10): PHOSPHORYLATION; tyrosine at position 10 is phosphorylated
NSSVNEGSTYHK

SEQ ID NO: 341 - protein, 11 aa, Homo sapiens
  MOD_RES (5)..(5): PHOSPHORYLATION; tyrosine at position 5 is phosphorylated
YSRTYDETYQR

SEQ ID NO: 342 - protein, 16 aa, Homo sapiens
  MOD_RES (3)..(3): PHOSPHORYLATION; tyrosine at position 3 is phosphorylated
YQYGGLNSGRPVTPPR

SEQ ID NO: 343 - protein, 14 aa, Homo sapiens
  MOD_RES (7)..(7): PHOSPHORYLATION; tyrosine at position 7 is phosphorylated
ENCAPLYDDALFLR

SEQ ID NO: 344 - protein, 16 aa, Homo sapiens
  MOD_RES (9)..(9): PHOSPHORYLATION; tyrosine at position 9 is phosphorylated
AQSQEDGDYINANYIR

SEQ ID NO: 345 - protein, 16 aa, Homo sapiens
  MOD_RES (14)..(14): PHOSPHORYLATION; tyrosine at position 14 is phosphorylated
AQSQEDGDYINANYIR

SEQ ID NO: 346 - protein, 15 aa, Homo sapiens
  MOD_RES (8)..(8): PHOSPHORYLATION; tyrosine at position 8 is phosphorylated
SEYGNITYPPAMKNA

SEQ ID NO: 347 - protein, 27 aa, Homo sapiens
  MOD_RES (14)..(14): PHOSPHORYLATION; tyrosine at position 14 is phosphorylated
VELSEINGDAGSNYINASYIDGFKEPR

SEQ ID NO: 348 - protein, 27 aa, Homo sapiens
  MOD_RES (19)..(19): PHOSPHORYLATION; tyrosine at position 19 is phosphorylated
VELSEINGDAGSNYINASYIDGFKEPR

SEQ ID NO: 349 - protein, 15 aa, Homo sapiens
  MOD_RES (14)..(14): PHOSPHORYLATION; tyrosine at position 14 is phosphorylated
VFPPFHLVNTATEYR

SEQ ID NO: 350 - protein, 36 aa, Homo sapiens
  MOD_RES (15)..(15): PHOSPHORYLATION; tyrosine at position 15 is phosphorylated
GLPSNNSLDFLDEEYEVPDLDGTRAPINGCCSLDAE

SEQ ID NO: 351 - protein, 30 aa, Homo sapiens
  MOD_RES (12)..(12): PHOSPHORYLATION; tyrosine at position 12 is phosphorylated
RYVENPNIMACYNELLQLEFGEVRSQLKLR

SEQ ID NO: 352 - protein, 27 aa, Homo sapiens
  MOD_RES (16)..(16): PHOSPHORYLATION; tyrosine at position 16 is phosphorylated
KLAEGSAYEEVPTSMMYSENDISNSIK

SEQ ID NO: 353 - protein, 30 aa, Homo sapiens
  MOD_RES (14)..(14): PHOSPHORYLATION; tyrosine at position 14 is phosphorylated
AVRQKEDCCFTILYGTQFVLSTLSLAADSK

SEQ ID NO: 354 - protein, 13 aa, Homo sapiens
  MOD_RES (8)..(8): PHOSPHORYLATION; tyrosine at position 8 is phosphorylated
KREGSDSYAITFR

SEQ ID NO: 355 - protein, 28 aa, Homo sapiens
  MOD_RES (11)..(11): PHOSPHORYLATION; tyrosine at position 11 is phosphorylated
SAADWSAGIKYHEESIHAAYVHVIENSR

SEQ ID NO: 356 - protein, 28 aa, Homo sapiens
  MOD_RES (20)..(20): PHOSPHORYLATION; tyrosine at position 20 is phosphorylated
SAADWSAGIKYHEESIHAAYVHVIENSR

SEQ ID NO: 357 - protein, 10 aa, Homo sapiens
  MOD_RES (6)..(6): PHOSPHORYLATION; tyrosine at position 6 is phosphorylated
TPIYPYLLPK

SEQ ID NO: 358 - protein, 8 aa, Homo sapiens
  MOD_RES (7)..(7): PHOSPHORYLATION; tyrosine at position 7 is phosphorylated
VSNSKFYS

SEQ ID NO: 359 - protein, 15 aa, Homo sapiens
  MOD_RES (8)..(8): PHOSPHORYLATION; tyrosine at position 8 is phosphorylated
ENWGNKGYILMARNK

SEQ ID NO: 360 - protein, 23 aa, Homo sapiens
  MOD_RES (3)..(3): PHOSPHORYLATION; tyrosine at position 3 is phosphorylated
DGYKHIHYIKDTVENAIQITSGK

SEQ ID NO: 361 - protein, 23 aa, Homo sapiens
  MOD_RES (8)..(8): PHOSPHORYLATION; tyrosine at position 8 is phosphorylated
DGYKHIHYIKDTVENAIQITSGK

SEQ ID NO: 362 - protein, 19 aa, Homo sapiens
  MOD_RES (12)..(12): PHOSPHORYLATION; tyrosine at position 12 is phosphorylated
QLAEEYLYRYGYTRVAEMR

SEQ ID NO: 363 - protein, 15 aa, Homo sapiens
  MOD_RES (3)..(3): PHOSPHORYLATION; tyrosine at position 3 is phosphorylated
SEYDRGVNTFSPEGR

SEQ ID NO: 364 - protein, 20 aa, Homo sapiens
  MOD_RES (18)..(18): PHOSPHORYLATION; tyrosine at position 18 is phosphorylated
VNNSTMLGASGDYADFQYLK

SEQ ID NO: 365 - protein, 12 aa, Homo sapiens
  MOD_RES (1)..(1): PHOSPHORYLATION; tyrosine at position 1 is phosphorylated
YYIHDHFIPIEK

SEQ ID NO: 366 - protein, 12 aa, Homo sapiens
  MOD_RES (2)..(2): PHOSPHORYLATION; tyrosine at position 2 is phosphorylated
YYIHDHFIPIEK

SEQ ID NO: 367 - protein, 15 aa, Homo sapiens
  MOD_RES (8)..(8): PHOSPHORYLATION; tyrosine at position 8 is phosphorylated
SSQAQMDYRRLQPSC

SEQ ID NO: 368 - protein, 35 aa, Homo sapiens
  MOD_RES (1)..(1): PHOSPHORYLATION; tyrosine at position 1 is phosphorylated
YKSLVTGTRARGVIAVLWVLAFGIGLTPFLGWNSK

SEQ ID NO: 369 - protein, 14 aa, Homo sapiens
  MOD_RES (1)..(1): PHOSPHORYLATION; tyrosine at position 1 is phosphorylated
YSDLDFEKVMHTRK

SEQ ID NO: 370 - protein, 23 aa, Homo sapiens
  MOD_RES (18)..(18): PHOSPHORYLATION; tyrosine at position 18 is phosphorylated
CCLLEGAGGAEEEPLDYYATALK

SEQ ID NO: 371 - protein, 35 aa, Homo sapiens
  MOD_RES (18)..(18): PHOSPHORYLATION; tyrosine at position 18 is phosphorylated
MAVLIFTDFICMAPISFYALSAILNKPLITVSNSK

SEQ ID NO: 372 - protein, 31 aa, Homo sapiens
  MOD_RES (27)..(27): PHOSPHORYLATION; tyrosine at position 27 is phosphorylated
PERALWQADTQVTSGVVTFTLQQLNAYNRTR

SEQ ID NO: 373 - protein, 10 aa, Homo sapiens
  MOD_RES (4)..(4): PHOSPHORYLATION; tyrosine at position 4 is phosphorylated
GILYAAPQLR

SEQ ID NO: 374 - protein, 34 aa, Homo sapiens
  MOD_RES (14)..(14): PHOSPHORYLATION; tyrosine at position 14 is phosphorylated
ASDKQTLLPNDQLYQPLKDREDDQYSHLQGNQLR

SEQ ID NO: 375 - protein, 34 aa, Homo sapiens; identical to SEQ ID NO: 374
  MOD_RES (25)..(25): PHOSPHORYLATION; tyrosine at position 25 is phosphorylated

SEQ ID NO: 376 - protein, 27 aa, Homo sapiens
  MOD_RES (9)..(9): PHOSPHORYLATION; tyrosine at position 9 is phosphorylated
AGMEEDHTYEGLDIDQTATYEDIVTLR

SEQ ID NO: 377 - protein, 27 aa, Homo sapiens
  MOD_RES (20)..(20): PHOSPHORYLATION; tyrosine at position 20 is phosphorylated
AGMEEDHTYEGLDIDQTATYEDIVTLR

SEQ ID NO: 378 - protein, 22 aa, Homo sapiens
  MOD_RES (21)..(21): PHOSPHORYLATION; tyrosine at position 21 is phosphorylated
YSSCPEPIVPGGYKIRGSTPYR

SEQ ID NO: 379 - protein, 15 aa, Homo sapiens
  MOD_RES (8)..(8): PHOSPHORYLATION; tyrosine at position 8 is phosphorylated
YEKSDGVYTGLSTRN

SEQ ID NO: 380 - protein, 18 aa, Homo sapiens
  MOD_RES (9)..(9): PHOSPHORYLATION; tyrosine at position 9 is phosphorylated
TPLTDTSVYTELPNAEPR

SEQ ID NO: 381 - protein, 21 aa, Homo sapiens
  MOD_RES (8)..(8): PHOSPHORYLATION; tyrosine at position 8 is phosphorylated
FLFYTNLYCSILFLTCISVHR

SEQ ID NO: 382 - protein, 14 aa, Homo sapiens
  MOD_RES (5)..(5): PHOSPHORYLATION; tyrosine at position 5 is phosphorylated
FSEIYHNINADQSK

SEQ ID NO: 383 - protein, 19 aa, Homo sapiens
  MOD_RES (5)..(5): PHOSPHORYLATION; tyrosine at position 5 is phosphorylated
LKLVYDKCDRSCKIQKKNR

SEQ ID NO: 384 - protein, 15 aa, Homo sapiens
  MOD_RES (13)..(13): PHOSPHORYLATION; tyrosine at position 13 is phosphorylated
LLLSIPHSDLLDYPK

SEQ ID NO: 385 - protein, 27 aa, Homo sapiens
  MOD_RES (6)..(6): PHOSPHORYLATION; tyrosine at position 6 is phosphorylated
RQDEDYDEQVEESLQDEDDNDVYILTK

SEQ ID NO: 386 - protein, 26 aa, Homo sapiens
  MOD_RES (2)..(2): PHOSPHORYLATION; tyrosine at position 2 is phosphorylated
KYLSLSPWDKATLSMGRLVLSNKMAR

SEQ ID NO: 387 - protein, 32 aa, Homo sapiens
  MOD_RES (1)..(1): PHOSPHORYLATION; tyrosine at position 1 is phosphorylated
YYTALYRKMLDPGLMTCSKQAMFLNLVYKSLK

SEQ ID NO: 388 - protein, 32 aa, Homo sapiens; identical to SEQ ID NO: 387
  MOD_RES (2)..(2): PHOSPHORYLATION; tyrosine at position 2 is phosphorylated

SEQ ID NO: 389 - protein, 32 aa, Homo sapiens; identical to SEQ ID NO: 387
  MOD_RES (6)..(6): PHOSPHORYLATION; tyrosine at position 6 is phosphorylated

SEQ ID NO: 390 - protein, 32 aa, Homo sapiens; identical to SEQ ID NO: 387
  MOD_RES (28)..(28): PHOSPHORYLATION; tyrosine at position 28 is phosphorylated

SEQ ID NO: 391 - protein, 14 aa, Homo sapiens
  MOD_RES (5)..(5): PHOSPHORYLATION; tyrosine at position 5 is phosphorylated
GTSQYYPSYSGSSR

SEQ ID NO: 392 - protein, 19 aa, Homo sapiens
  MOD_RES (16)..(16): PHOSPHORYLATION; tyrosine at position 16 is phosphorylated
YYYEKGIMQKVAGERYVYK

SEQ ID NO: 393 - protein, 20 aa, Homo sapiens
  MOD_RES (16)..(16): PHOSPHORYLATION; tyrosine at position 16 is phosphorylated
IGGDAGTSLNSNDYGYGGQK

SEQ ID NO: 394 - protein, 25 aa, Homo sapiens
  MOD_RES (21)..(21): PHOSPHORYLATION; tyrosine at position 21 is phosphorylated
IDSIPHLNNSTPLVDPSVYGYGVQK

SEQ ID NO: 395 - protein, 14 aa, Homo sapiens
  MOD_RES (9)..(9): PHOSPHORYLATION; tyrosine at position 9 is phosphorylated
LSVKEDPSYDSVRR

SEQ ID NO: 396 - protein, 16 aa, Homo sapiens
  MOD_RES (1)..(1): PHOSPHORYLATION; tyrosine at position 1 is phosphorylated
YYTSYKDAYIGLCLPK

SEQ ID NO: 397 - protein, 16 aa, Homo sapiens
  MOD_RES (2)..(2): PHOSPHORYLATION; tyrosine at position 2 is phosphorylated
YYTSYKDAYIGLCLPK

SEQ ID NO: 398 - protein, 16 aa, Homo sapiens
  MOD_RES (5)..(5): PHOSPHORYLATION; tyrosine at position 5 is phosphorylated
YYTSYKDAYIGLCLPK

SEQ ID NO: 399 - protein, 29 aa, Homo sapiens
  MOD_RES (21)..(21): PHOSPHORYLATION; tyrosine at position 21 is phosphorylated
PSFSMAHLDGNTEPGLTLGGYFCPQCRAK

SEQ ID NO: 400 - protein, 19 aa, Homo sapiens
  MOD_RES (13)..(13): PHOSPHORYLATION; tyrosine at position 13 is phosphorylated
FDLVDAYGTEPPYTYSGKR

SEQ ID NO: 401 - protein, 19 aa, Homo sapiens
  MOD_RES (15)..(15): PHOSPHORYLATION; tyrosine at position 15 is phosphorylated
FDLVDAYGTEPPYTYSGKR

SEQ ID NO: 402 - protein, 36 aa, Homo sapiens
  MOD_RES (20)..(20): PHOSPHORYLATION; tyrosine at position 20 is phosphorylated
IYEVCSNGPAPTDSQPPEDYSFGAGEEEEEEEELQR

SEQ ID NO: 403 - protein, 27 aa, Homo sapiens
  MOD_RES (15)..(15): PHOSPHORYLATION; tyrosine at position 15 is phosphorylated
CAGCGGKIADRFLLYAMDSYWHSRCLK

SEQ ID NO: 404 - protein, 27 aa, Homo sapiens
  MOD_RES (20)..(20): PHOSPHORYLATION; tyrosine at position 20 is phosphorylated
CAGCGGKIADRFLLYAMDSYWHSRCLK

SEQ ID NO: 405 - protein, 16 aa, Homo sapiens
  MOD_RES (12)..(12): PHOSPHORYLATION; tyrosine at position 12 is phosphorylated
MNIQMLLEAADYLERR

SEQ ID NO: 406 - protein, 34 aa, Homo sapiens
  MOD_RES (18)..(18): PHOSPHORYLATION; tyrosine at position 18 is phosphorylated
QGGYSQSNYNSPGSGQNYSGPPSSYQSSQGGYGR

SEQ ID NO: 407 - protein, 19 aa, Homo sapiens
  MOD_RES (15)..(15): PHOSPHORYLATION; tyrosine at position 15 is phosphorylated
IPLPGAEMLEEEPLYVNAK

SEQ ID NO: 408 - protein, 14 aa, Homo sapiens
  MOD_RES (9)..(9): PHOSPHORYLATION; tyrosine at position 9 is phosphorylated
HHAQETLHYQNLAK

SEQ ID NO: 409 - protein, 23 aa, Homo sapiens
  MOD_RES (17)..(17): PHOSPHORYLATION; tyrosine at position 17 is phosphorylated
FGKVTSVQIHGTSEERYGLVFFR

SEQ ID NO: 410 - protein, 15 aa, Homo sapiens
  MOD_RES (6)..(6): PHOSPHORYLATION; tyrosine at position 6 is phosphorylated
QMQVLYGQHFPIEVR

SEQ ID NO: 411 - protein, 10 aa, Homo sapiens
  MOD_RES (4)..(4): PHOSPHORYLATION; tyrosine at position 4 is phosphorylated
LGHYATQLQK

SEQ ID NO: 412 - protein, 8 aa, Homo sapiens
  MOD_RES (4)..(4): PHOSPHORYLATION; tyrosine at position 4 is phosphorylated
HILYNEQR

SEQ ID NO: 413 - protein, 26 aa, Homo sapiens
  MOD_RES (17)..(17): PHOSPHORYLATION; tyrosine at position 17 is phosphorylated
GLSTPNTIDQWDTTGLYSFSEQTRSL

SEQ ID NO: 414 - protein, 22 aa, Homo sapiens
  MOD_RES (4)..(4): PHOSPHORYLATION; tyrosine at position 4 is phosphorylated
VPSYDSFDSEDYPAALPNHKPK

SEQ ID NO: 415 - protein, 10 aa, Homo sapiens
  MOD_RES (8)..(8): PHOSPHORYLATION; tyrosine at position 8 is phosphorylated
YAADRNHYRR

SEQ ID NO: 416 - protein, 29 aa, Homo sapiens
  MOD_RES (2)..(2): PHOSPHORYLATION; tyrosine at position 2 is phosphorylated
NYQQNYQNSESGEKNEGSESAPEGQAQQR

SEQ ID NO: 417 - protein, 30 aa, Homo sapiens
  MOD_RES (4)..(4): PHOSPHORYLATION; tyrosine at position 4 is phosphorylated
RPQYSNPPVQGEVMEGADNQGAGEQGRPVR

SEQ ID NO: 418 - protein, 9 aa, Homo sapiens
  MOD_RES (6)..(6): PHOSPHORYLATION; tyrosine at position 6 is phosphorylated
EVDAEYEAR

SEQ ID NO: 419 - protein, 22 aa, Homo sapiens
  MOD_RES (7)..(7): PHOSPHORYLATION; tyrosine at position 7 is phosphorylated
SEDPDYYQYNIQGSHHSSEGNE

SEQ ID NO: 420 - protein, 11 aa, Homo sapiens
  MOD_RES (4)..(4): PHOSPHORYLATION; tyrosine at position 4 is phosphorylated
APSYLEISSMR

SEQ ID NO: 421 - protein, 12 aa, Homo sapiens
  MOD_RES (10)..(10): PHOSPHORYLATION; tyrosine at position 10 is phosphorylated
AADKLAPAQYIR

SEQ ID NO: 422 - protein, 22 aa, Homo sapiens
  MOD_RES (14)..(14): PHOSPHORYLATION; tyrosine at position 14 is phosphorylated
GMDSGFAGGEDEIYNVYDQAWR

SEQ ID NO: 423 - protein, 22 aa, Homo sapiens
  MOD_RES (17)..(17): PHOSPHORYLATION; tyrosine at position 17 is phosphorylated
GMDSGFAGGEDEIYNVYDQAWR

SEQ ID NO: 424 - protein, 16 aa, Homo sapiens
  MOD_RES (3)..(3): PHOSPHORYLATION; tyrosine at position 3 is phosphorylated
NIYSNSQGKPGSPEPE

SEQ ID NO: 425 - protein, 17 aa, Homo sapiens
  MOD_RES (10)..(10): PHOSPHORYLATION; tyrosine at position 10 is phosphorylated
LCGKPDVPIYDGSWVEW

SEQ ID NO: 426 - protein, 15 aa, Homo sapiens
  MOD_RES (2)..(2): PHOSPHORYLATION; tyrosine at position 2 is phosphorylated
MYHHTIPVISLYSLR

SEQ ID NO: 427 - protein, 23 aa, Homo sapiens
  MOD_RES (11)..(11): PHOSPHORYLATION; tyrosine at position 11 is phosphorylated
VPFLDHRFSSYYLSLPPEMRIPK

SEQ ID NO: 428 - protein, 23 aa, Homo sapiens
  MOD_RES (12)..(12): PHOSPHORYLATION; tyrosine at position 12 is phosphorylated
VPFLDHRFSSYYLSLPPEMRIPK

SEQ ID NO: 429 - protein, 12 aa, Homo sapiens
  MOD_RES (3)..(3): PHOSPHORYLATION; tyrosine at position 3 is phosphorylated
LHYAVELFEGLK

SEQ ID NO: 430 - protein, 16 aa, Homo sapiens
  MOD_RES (6)..(6): PHOSPHORYLATION; tyrosine at position 6 is phosphorylated
YMLVRYEDLARNPMKK

SEQ ID NO: 431 - protein, 11 aa, Homo sapiens
  MOD_RES (7)..(7): PHOSPHORYLATION; tyrosine at position 7 is phosphorylated
PPYTVVYFPVR

SEQ ID NO: 432 - protein, 24 aa, Homo sapiens
  MOD_RES (17)..(17): PHOSPHORYLATION; tyrosine at position 17 is phosphorylated
HVSDLKFAMKCYTPLVYKGITPCK

SEQ ID NO: 433 - protein, 26 aa, Homo sapiens
  MOD_RES (23)..(23): PHOSPHORYLATION; tyrosine at position 23 is phosphorylated
TAYCQDLFSETELAFLVSGAILYGCY

SEQ ID NO: 434 - protein, 26 aa, Homo sapiens
  MOD_RES (26)..(26): PHOSPHORYLATION; tyrosine at position 26 is phosphorylated
TAYCQDLFSETELAFLVSGAILYGCY

SEQ ID NO: 435 - protein, 9 aa, Homo sapiens
  MOD_RES (6)..(6): PHOSPHORYLATION; tyrosine at position 6 is phosphorylated
LEQDEYALR

SEQ ID NO: 436 - protein, 18 aa, Homo sapiens
  MOD_RES (12)..(12): PHOSPHORYLATION; tyrosine at position 12 is phosphorylated
LKFGIGDGNLQYYLYNWK

SEQ ID NO: 437 - protein, 31 aa, Homo sapiens
  MOD_RES (8)..(8): PHOSPHORYLATION; tyrosine at position 8 is phosphorylated
EIILVDDYSNDPEDGALLGKIEKVRVLRNDR

SEQ ID NO: 438 - protein, 23 aa, Homo sapiens
  MOD_RES (9)..(9): PHOSPHORYLATION; tyrosine at position 9 is phosphorylated
DGTLDDGEYNPTDDRPSRADQFE

SEQ ID NO: 439 - protein, 19 aa, Homo sapiens
  MOD_RES (14)..(14): PHOSPHORYLATION; tyrosine at position 14 is phosphorylated
MLSQPLKDSDAEVYSIIKK

SEQ ID NO: 440 - protein, 18 aa, Homo sapiens
  MOD_RES (14)..(14): PHOSPHORYLATION; tyrosine at position 14 is phosphorylated
MLAQPLKDSDVEVYNIIK

SEQ ID NO: 441 - protein, 16 aa, Homo sapiens
  MOD_RES (11)..(11): PHOSPHORYLATION; tyrosine at position 11 is phosphorylated
DNVRTIGAQVYEQVLR

SEQ ID NO: 442 - protein, 23 aa, Homo sapiens
  MOD_RES (1)..(1): PHOSPHORYLATION; tyrosine at position 1 is phosphorylated
YMYLLPYTEFFGGVSGLTVEQFR

SEQ ID NO: 443 - protein, 23 aa, Homo sapiens
  MOD_RES (3)..(3): PHOSPHORYLATION; tyrosine at position 3 is phosphorylated
YMYLLPYTEFFGGVSGLTVEQFR

SEQ ID NO: 444 - protein, 23 aa, Homo sapiens
  MOD_RES (7)..(7): PHOSPHORYLATION; tyrosine at position 7 is phosphorylated
YMYLLPYTEFFGGVSGLTVEQFR

SEQ ID NO: 445 - protein, 31 aa, Homo sapiens
  MOD_RES (14)..(14): PHOSPHORYLATION; tyrosine at position 14 is phosphorylated
TVAISDAAQLPHDYCTTPGGTLFSTTPGGTR

SEQ ID NO: 446 - protein, 22 aa, Homo sapiens
  MOD_RES (14)..(14): PHOSPHORYLATION; tyrosine at position 14 is phosphorylated
VIASTDVDKTGASYYGEQTLHY

SEQ ID NO: 447 - protein, 24 aa, Homo sapiens
  MOD_RES (8)..(8): PHOSPHORYLATION; tyrosine at position 8 is phosphorylated
SQGLSQLYHNQSQGLLSQLQGQSK

SEQ ID NO: 448 - protein, 14 aa, Homo sapiens
  MOD_RES (9)..(9): PHOSPHORYLATION; tyrosine at position 9 is phosphorylated
VFDHKQGTYGGYFR

SEQ ID NO: 449 - protein, 20 aa, Homo sapiens
  MOD_RES (14)..(14): PHOSPHORYLATION; tyrosine at position 14 is phosphorylated
LGHVVMGNNAVSPYQQVIEK

SEQ ID NO: 450 - protein, 17 aa, Homo sapiens
  MOD_RES (7)..(7): PHOSPHORYLATION; tyrosine at position 7 is phosphorylated
FETSKYYVTIIDAPGHR

SEQ ID NO: 451 - protein, 14 aa, Homo sapiens
  MOD_RES (2)..(2): PHOSPHORYLATION; tyrosine at position 2 is phosphorylated
FYEQMNGPVAGASR

SEQ ID NO: 452 - protein, 18 aa, Homo sapiens
  MOD_RES (5)..(5): PHOSPHORYLATION; tyrosine at position 5 is phosphorylated
CELLYEGPPDDEAAMGIK

SEQ ID NO: 453 - protein, 26 aa, Homo sapiens
  MOD_RES (12)..(12): PHOSPHORYLATION; tyrosine at position 12 is phosphorylated
AAYDPYAYPSDYDMHTGDPKQDLAYE

SEQ ID NO: 454 - protein, 16 aa, Homo sapiens
  MOD_RES (9)..(9): PHOSPHORYLATION; tyrosine at position 9 is phosphorylated
YDDRGSRDYDRGYDSR

SEQ ID NO: 455 - protein, 18 aa, Homo sapiens
  MOD_RES (15)..(15): PHOSPHORYLATION; tyrosine at position 15 is phosphorylated
DRYDDRGSRDYDRGYDSR

SEQ ID NO: 456 - protein, 21 aa, Homo sapiens
  MOD_RES (4)..(4): PHOSPHORYLATION; tyrosine at position 4 is phosphorylated
DDDYRGGGDRYEDRYDRRDDR

SEQ ID NO: 457 - protein, 21 aa, Homo sapiens
  MOD_RES (11)..(11): PHOSPHORYLATION; tyrosine at position 11 is phosphorylated
DDDYRGGGDRYEDRYDRRDDR

SEQ ID NO: 458 - protein, 24 aa, Homo sapiens
  MOD_RES (6)..(6): PHOSPHORYLATION; tyrosine at position 6 is phosphorylated
SSASKYAALSVDGEDENEGEDYAE

SEQ ID NO: 459 - protein, 24 aa, Homo sapiens
  MOD_RES (22)..(22): PHOSPHORYLATION; tyrosine at position 22 is phosphorylated
SSASKYAALSVDGEDENEGEDYAE

SEQ ID NO: 460 - protein, 13 aa, Homo sapiens
  MOD_RES (3)..(3): PHOSPHORYLATION; tyrosine at position 3 is phosphorylated
VVYSKAASVPKVE

SEQ ID NO: 461 - protein, 13 aa, Homo sapiens
  MOD_RES (4)..(4): PHOSPHORYLATION; tyrosine at position 4 is phosphorylated
LTRGEADRDTYRR

SEQ ID NO: 462 - protein, 18 aa, Homo sapiens
  MOD_RES (15)..(15): PHOSPHORYLATION; tyrosine at position 15 is phosphorylated
SGYGQTTDSSYGQNYSGY

SEQ ID NO: 463 - protein, 20 aa, Homo sapiens
  MOD_RES (13)..(13): PHOSPHORYLATION; tyrosine at position 13 is phosphorylated
DQHQGSYDEQSNYDQQHDSY

SEQ ID NO: 464 - protein, 27 aa, Homo sapiens
  MOD_RES (20)..(20): PHOSPHORYLATION; tyrosine at position 20 is phosphorylated
DQHQGSYDEQSNYDQQHDSYSQNQQSY

SEQ ID NO: 465 - protein, 25 aa, Homo sapiens
  MOD_RES (21)..(21): PHOSPHORYLATION; tyrosine at position 21 is phosphorylated
LRRPIGKMTITEQKYEGEYRYVNSR

SEQ ID NO: 466 - protein, 13 aa, Homo sapiens
  MOD_RES (11)..(11): PHOSPHORYLATION; tyrosine at position 11 is phosphorylated
LAQLQEGQDLYSR

SEQ ID NO: 467 - protein, 21 aa, Homo sapiens
  MOD_RES (2)..(2): PHOSPHORYLATION; tyrosine at position 2 is phosphorylated
EYSPAATTAENGGGKKKQKEK

SEQ ID NO: 468 - protein, 26 aa, Homo sapiens
  MOD_RES (23)..(23): PHOSPHORYLATION; tyrosine at position 23 is phosphorylated
VKYLLTPREPNRWAVEREGATPYNSR

SEQ ID NO: 469 - protein, 27 aa, Homo sapiens
  MOD_RES (8)..(8): PHOSPHORYLATION; tyrosine at position 8 is phosphorylated
NVEFEPTYEEFPSLESDSLLDYSCMQR

SEQ ID NO: 470 - protein, 27 aa, Homo sapiens
  MOD_RES (22)..(22): PHOSPHORYLATION; tyrosine at position 22 is phosphorylated
NVEFEPTYEEFPSLESDSLLDYSCMQR

SEQ ID NO: 471 - protein, 35 aa, Homo sapiens
  MOD_RES (1)..(1): PHOSPHORYLATION; tyrosine at position 1 is phosphorylated
YFSFPGELLMRMLQMLVLPLIVSSLVTGMASLDNK

SEQ ID NO: 472 - protein, 28 aa, Homo sapiens
  MOD_RES (7)..(7): PHOSPHORYLATION; tyrosine at position 7 is phosphorylated
SNETNGYLDSAQAGPAAGPGAPGTAAGR

SEQ ID NO: 473 - protein, 20 aa, Homo sapiens
  MOD_RES (9)..(9): PHOSPHORYLATION; tyrosine at position 9 is phosphorylated
DENNEEDAYPDNVHAAAEEK

SEQ ID NO: 474 - protein, 16 aa, Homo sapiens
  MOD_RES (9)..(9): PHOSPHORYLATION; tyrosine at position 9 is phosphorylated
NQSTTYPVYTESTDDK

SEQ ID NO: 475 - protein, 15 aa, Homo sapiens
  MOD_RES (8)..(8): PHOSPHORYLATION; tyrosine at position 8 is phosphorylated
STEEQQLYWAKGTGF

SEQ ID NO: 476 - protein, 26 aa, Homo sapiens
  MOD_RES (21)..(21): PHOSPHORYLATION; tyrosine at position 21 is phosphorylated
FSADTDILLGGLGLFGGRGEYTAKIK

SEQ ID NO: 477 - protein, 15 aa, Homo sapiens
  MOD_RES (8)..(8): PHOSPHORYLATION; tyrosine at position 8 is phosphorylated
LQKLPFYDLLDELIK

SEQ ID NO: 478 - protein, 20 aa, Homo sapiens
  MOD_RES (16)..(16): PHOSPHORYLATION; tyrosine at position 16 is phosphorylated
PSLVRDLLDDILPLLYQETK

SEQ ID NO: 479 - protein, 29 aa, Homo sapiens
  MOD_RES (3)..(3): PHOSPHORYLATION; tyrosine at position 3 is phosphorylated
LTYARLAQLLEGYARYSVSVFQPPFQPGR

SEQ ID NO: 480 - protein, 28 aa, Homo sapiens
  MOD_RES (9)..(9): PHOSPHORYLATION; tyrosine at position 9 is phosphorylated
FLTARDKAYKAVVNDATIFKLELPLKQK